(19) United States
(12) Patent Application Publication
(10) Pub. No.: US 2021/0380535 A1
(43) Pub. Date: Dec. 9, 2021

(54) CO-CRYSTALS COMPRISING EPICATECHIN AND A CARBOXY-N-HETEROCYCLIC CO-CRYSTAL FORMER

(71) Applicant: Epirium Bio Inc., San Diego, CA (US)

(72) Inventors: Sundeep DUGAR, San Jose, CA (US); Somdutta SEN, Haryana (IN); Bilash KUILA, Haryana (IN)

(21) Appl. No.: 17/288,371

(22) PCT Filed: Oct. 24, 2019
(86) PCT No.: PCT/US2019/057930

§ 371 Date: Apr. 23, 2021

Related U.S. Application Data
(60) Provisional application No. 62/750,182, filed on Oct. 24, 2018.

(51) Int. Cl. C07D 213/80; C07D 311/62; C07D 207/16

(52) U.S. Cl. CPC C07D 213/80; C07B 2200/13; C07D 207/16; C07D 311/62

(57) ABSTRACT

The invention provides novel co-crystals of epicatechin with a carboxy-N-heterocyclic co-crystal former such as trigonelline or proline and their pharmaceutical compositions. Methods of preparing the co-crystals and methods of using them are also provided.

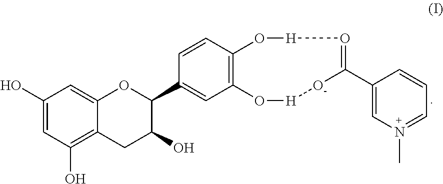

### CROSS REFERENCE TO RELATED APPLICATION

This application claims priority to U.S. Provisional Application Ser. No. 62/750,182, filed Oct. 24, 2018, which is hereby incorporated by reference in its entirety.

### FIELD OF THE INVENTION

The present invention pertains to field of crystals. More particularly, the present invention is drawn to a novel co-crystal of epicatechin with a co-crystal former.

### BACKGROUND OF THE INVENTION

Co-crystals have generated tremendous interest in pharmaceutical research and development because of the potential to customize physicochemical properties of the solid while maintaining the chemical integrity of the drug. Co-crystals are part of a broader class of multicomponent crystals, where two or more molecules (commonly referred to as drug and co-former) populate a homogeneous crystalline lattice in a well-defined stoichiometry. What distinguishes co-crystals from other types of multi component crystals such as salts and solvates is that drug and co-former are solids at ambient temperature and that the intermolecular interactions are nonionic in nature. The diversity of solid forms that can be generated from a drug greatly increases through co-crystallization; the physicochemical properties of the co-crystals can vary depending on the characteristics of its constituent molecules. Pharmaceutically relevant properties that can change via co-crystallization include but are not limited to solubility, dissolution, moisture uptake, chemical stability, mechanical properties, and bioavailability.

The main advantage of co-crystals is the ability to generate a variety of solid forms of a drug that have physicochemical properties distinct from the solid co-crystal components. Such properties include but are not limited to solubility, dissolution, bioavailability, hygroscopicity, hydrate/solvate formation, crystal morphology, fusion properties, chemical and thermal stability, and mechanical properties. These properties can directly or indirectly affect the suitability of a particular API as a pharmaceutical product.

A co-crystal of a drug (an active nutraceutical ingredient or an active pharmaceutical ingredient) is a distinct chemical composition between the drug and co-former, and generally possesses distinct crystallographic and spectroscopic properties when compared to those of the drug and a co-former individually. Unlike salts, which possess a neutral net charge, but which are comprised of charge-balanced components, co-crystals are comprised of neutral species. Thus, unlike a salt, one cannot determine the stoichiometry of a co-crystal based on charge balance. Indeed, one can often obtain co-crystals having stoichiometric ratios of drug to co-former of greater than or less than 1:1. The stoichiometric ratio of an API to a co-former is a generally unpredictable feature of a co-crystal.

Several co-crystal compounds, composition, methods of preparations and uses thereof have been known in the prior art. For instance, WO2017001991 relates to certain crystalline compounds containing Trigoneline and a cocrystal former.

WO 2009/136408 relates to a pharmaceutical co-crystal comprising soluble forms of broad-spectrum fluoroquinolone antibacterial agents namely Ciprofloxacin and Norfloxacin with small molecules that have unique physical properties and biological activity which differ from the active agent in pure form, to process for preparation of the same and also relates to pharmaceutical compositions comprising these synergistic co-crystals.

WO 2017/001991 discloses co-crystals of certain flavonoids with trigonelline. However, it does not disclose co-crystals of epicatechin.

However, the inventors in their earlier applications incorporated herein by entirety note that epicatechin is preferred flavonol and have wide variety utility. Hence, it is needed to optimize the physiochemical properties of epicatechin.

The present invention discloses the modification of physicochemical properties of epicatechin through co-crystal formation.

### OBJECT OF THE INVENTION

An object of the present invention is to provide a co-crystal of epicatechin with a co-crystal former (e.g., trigonelline, proline), a process for preparation and composition comprising the co-crystal.

### SUMMARY OF THE INVENTION

The present invention relates to a novel co-crystal of epicatechin with a co-crystal former. In one aspect, the present invention discloses novel co-crystals of epicatechin:aco-crystal former of Formula (I):

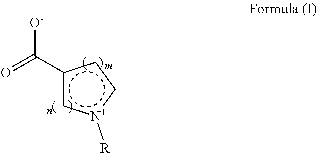

In another aspect, the present invention provides a method for preparation of a novel co-crystal of epicatechin:aco-crystal former of Formula (I). The present invention also discloses pharmaceutical compositions comprising co-crystal of epicatechin:aco-crystal former of Formula (I) along with other pharmaceutically acceptable excipients. The present invention also discloses a co-crystal of epicatechin:aco-crystal former of Formula (I) with improved physicochemical and biopharmaceutical properties and pharmacological activity. In some embodiments, a co-crystal former is trigonelline. In some embodiments, a co-crystal former is proline.

In yet another aspect, the co-crystals and the pharmaceutical compositions of the present invention are useful in treating diseases or disorders that would benefit from modification of Electron transfer Chain (ETC) and particularly electron transfer chain IV.

### BRIEF DESCRIPTION OF DRAWINGS

FIG. 1 shows a differential scanning calorimetry (DSC) pattern of Compound 101 prepared as described in Example 1.

FIG. 2 shows a differential scanning calorimetry (DSC) pattern of Compound 102 prepared as described in Example 1.

FIG. 3 shows a differential scanning calorimetry (DSC) pattern of Compound 103 prepared as described in Example 1.

FIG. 4 shows a differential scanning calorimetry (DSC) pattern of Compound 104 prepared as described in Example 1.

FIG. 5 shows a differential scanning calorimetry (DSC) pattern of Compound 105 prepared as described in Example 1.

FIG. 6 shows a differential scanning calorimetry (DSC) pattern of Compound 106 prepared as described in Example 1.

FIG. 7 shows a differential scanning calorimetry (DSC) pattern of Compound 107.

FIG. 8 shows a differential scanning calorimetry (DSC) pattern of Compound 108.

FIG. 9 shows a differential scanning calorimetry (DSC) pattern of trigonelline.

FIG. 10 shows an experimental X-ray powder diffraction (PXRD) pattern of Compound 101.

FIG. 11 shows an experimental X-ray powder diffraction (PXRD) pattern of Compound 104.

FIG. 12 shows an experimental X-ray powder diffraction (PXRD) pattern of Compound 106.

FIG. 13 shows an overlay infrared spectroscopy pattern of Compound 101, Compound 108, and trigonelline.

FIG. 14 shows plasma levels of (+) epicatechin (SPR590, Compound 108) and its 1:1 co-crystal with trigonelline (SPR515, Compound 104) at various time points.

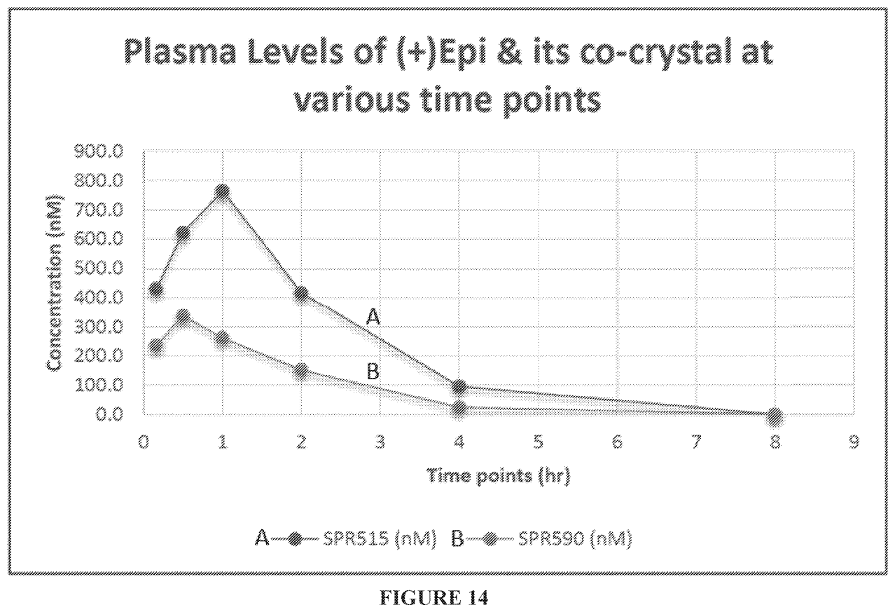

FIG. 15 shows a differential scanning calorimetry (DSC) pattern of Compound 101 prepared as described in Example 6.

FIG. 16 shows a differential scanning calorimetry (DSC) pattern of Compound 104 prepared as described in Example 7.

FIG. 17 shows a H NMR spectrum of Compound 104 in DMSO showing 1:1 stoichiometry of (+) epicatechin and trigonelline.

FIG. 18 shows a differential scanning calorimetry (DSC) pattern of Compound 109 prepared as described in Example 8.

FIG. 19 shows a differential scanning calorimetry (DSC) pattern of Compound 110 prepared as described in Example 9.

FIG. 20 shows an experimental X-ray powder diffraction (PXRD) pattern of Compound 108.

### DETAILED DESCRIPTION OF THE INVENTION

The present invention relates to a co-crystal comprising epicatechin with a co-crystal former of Formula (I), and processes for preparation thereof.

As used herein, the term “co-crystal” denotes crystalline molecular complexes, encompassing hydrates and solvates. “Co-crystals” are composed of multi-component, stoichiometric and neutral molecular species, each existing as a solid under ambient conditions.

Co-crystals exhibit properties different from free drugs or salts. The solid form influences relevant physico-chemical parameters such as solubility, dissolution rate of the drug, chemical stability, melting point, and hygroscopicity, which can result in solids with superior properties.

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, molar percent, or weight percent of ingredients of a composition or a dosage form, mean a dose, amount, molar percent, or weight percent that is recognized by those of ordinary skill in the art to provide a pharmacological effect equivalent to that obtained from the specified dose, amount, molar percent, or weight percent. Specifically, the terms “about” and “approximately,” when used in this context, contemplate a dose, amount, molar percent, or weight percent within 15%, within 10%, within 5%, within 4%, within 3%, within 2%, within 1%, or within 0.5% of the specified dose, amount, molar percent, or weight percent.

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition.

As used herein, the term “pharmaceutically acceptable excipient,” and cognates thereof, refers to adjuvants, binders, diluents, etc. known to the skilled artisan that are suitable for administration to an individual (e.g., a mammal or non-mammal). Combinations of two or more excipients are also contemplated. The pharmaceutically acceptable excipient(s) and any additional components, as described herein, should be compatible for use in the intended route of administration (e.g., oral, parenteral) for a particular dosage form, as would be recognized by the skilled artisan.

The terms “treat,” “treating,” and “treatment” are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, the term “substantially as shown in” when referring, for example, to an XRPD pattern or a DSC graph, includes a pattern or graph that is not necessarily identical to those depicted herein, but that falls within the limits of experimental error or deviations when considered by one of ordinary skill in the art.

### Co-Crystals

As used herein the co-crystals of the present invention include epicatechin and a co-crystal former of Formula (I):

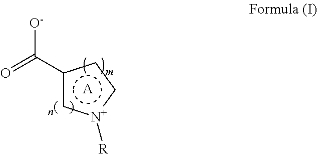

or a stereoisomer thereof, wherein

n is 0, 1, 2, or 3;

m is 0, 1, 2, 3, or 4;

[symbol] indicates that ring A is saturated, partially unsaturated, or fully unsaturated; and

R is hydrogen or alkyl, wherein the alkyl is unsubstituted or substituted with one or more substituents independently selected from the group consisting of halo, —CN, —OH, and haloalkyl.

The co-crystal former of Formula (I) may be in zwitter-ionic form or in a form in which all atoms have neutral charge. In some instances, the co-crystal former of Formula (I) may be depicted in a positively charged form, such as when the nitrogen of ring A is positively charged and the carboxylate is present in the COOH form. In some embodiments, the co-crystal former of Formula (I) may be depicted in a negatively charged form, such as when the carboxylate is present in in the COO⁻ form and the nitrogen of ring A is neutral.

In some embodiments, ring A is saturated. In some embodiments, ring A is fully unsaturated. In some embodiments, ring A is partially unsaturated. In some embodiments ring A is a pyridinium. In some embodiments, ring A is pyrrolidinium. In some embodiments, ring A is azetidinium.

In some embodiments, n is 0. In some embodiments, n is 1. In some embodiments, n is 2. In some embodiments, n is 3. In some embodiments, m is 0. In some embodiments, m is 1. In some embodiments, m is 2. In some embodiments, m is 4. In some embodiments, n is 1 and m is 2. In some embodiments, n is 0 and m is 2. In some embodiments, n is 1 and m is 0. In some embodiments, n is 0 or 1, and m is 1 or 2.

In some embodiments, n is 1, m is 2, and ring A is fully unsaturated (i.e., aromatic). In some embodiments, n is 0, m is 2, and ring A is saturated. In some embodiments, n is 1, m is 0, and ring A is saturated.

In some embodiments R is hydrogen. In some embodiments, the R is unsubstituted or substituted C₁-C₆ alkyl. In some embodiments, the R is alkyl substituted with one or more substituents independently selected from the group consisting of halo, —CN, —OH, and haloalkyl. In some embodiments, the R is C₁-C₆ alkyl substituted with one or more groups selected from the group consisting of halo, CN, —OH, and C₁-C₆ haloalkyl. In some embodiments, R is methyl. In some embodiments, R is ethyl.

In some embodiments, ring A is fully unsaturated, and R is hydrogen. In some embodiments, ring A is saturated and R is hydrogen. In some embodiments, ring A is fully unsaturated, and R is methyl. In some embodiments, ring A is saturated and R is methyl.

In some embodiments, the co-crystal former of Formula (I) is a co-crystal former of Formula (Ia):

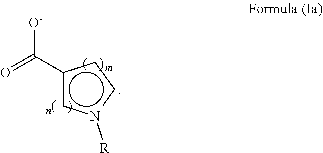

In some embodiments, the co-crystal former of Formula (I) is a co-crystal former of Formula (Ib):

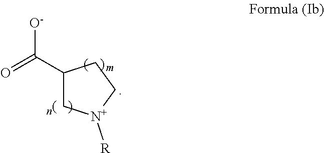

In some embodiments, the co-crystal former of Formula (I) is in an R stereochemical configuration. In some embodiments, the co-crystal former of Formula (I) is in an S stereochemical configuration. In some embodiments, the co-crystal former of Formula (I) is in an L stereochemical configuration. In some embodiments, the co-crystal former of Formula (I) is in a D stereochemical configuration.

In some embodiments, a co-crystal former is trigonelline. The structure of trigonelline, specifically used according to the present invention is shown below:

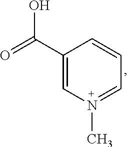

or a neutral (e.g., zwitterion) form thereof.

In some embodiments, a co-crystal former is proline. In some embodiments, the proline is D-proline. In some embodiments, the proline is L-proline. The structure of proline, specifically used according to the present invention is shown below:

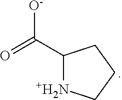

Without being bound by any particular theory, it is believed that epicatechin and the co-crystal former are bonded together through hydrogen bonds (e.g., via the alpha-carboxylic acid group of the co-crystal former). Other non-covalent interactions, including pi-stacking and van der Waals interactions, may also be present. It is also believed that the ring, either aromatic or non-aromatic, of the co-crystal former provides appropriate rigidness to form a co-crystal with (+) epicatechin or (−) epicatechin. To give an example, (+) epicatechin and trigonelline at a molar ratio of 1:1 may form a co-crystal as depicted below:

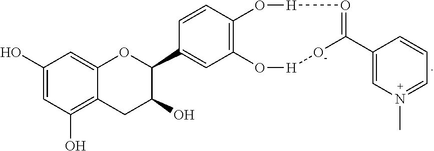

The present invention discloses a novel co-crystal of epicatechin:trigonelline in some embodiments. In other embodiments, a novel co-crystal of epicatechin:proline is disclosed. In some embodiments the proline is D-proline. In some embodiments, the proline is L-proline.

The epicatechin used in the present invention may be epicatechin, (+) epicatechin, (−) epicatechin or racemic mixture of epicatechin. The structures of (+) epicatechin and (−) epicatechin are shown below:

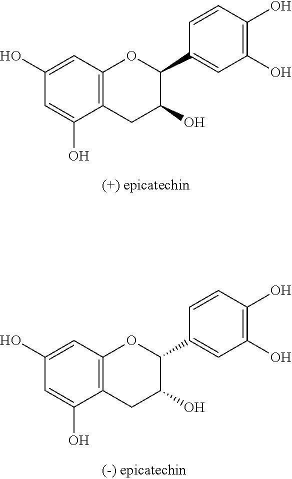 [SMILES: Oc1cc(O)c2c(c1)O[C@@H](c1ccc(O)c(O)c1)[C@@H](O)C2 ; Oc1cc(O)c2c(c1)O[C@H](c1ccc(O)c(O)c1)[C@H](O)C2]

In some embodiments, epicatechin is enantiomercally pure or enatiomerically enriched. In some embodiments, the epicatechin is enatiomerically pure or enatiomerically enriched (+) epicatechin. In other embodiments, the epicatechin is enatiomerically pure or enatiomerically enriched (−) epicatechin. The purity of the enatiomerically pure or enatiomerically enriched (+)/(−) epicatechin is at least 80%, 85%, 90%, 91%, 92%, 93%, 94%, 95%, 96%, 97%, 98%, 99%, 99.5%, 99.8%, 99.9%, or 100%.

Co-crystals described herein can have a purity of at least 80%, 85%, 90%, 91%, 92%, 93%, 94%, 95%, 96%, 97%, 98%, 99%, 99.5%, 99.8%, or 99.9% In some embodiments, provided is a co-crystal of (+) epicatechin:trigonelline. In some embodiments, provided is a co-crystal of (−) epicatechin:trigonelline. In some embodiments, provided is a co-crystal of (+) epicatechin:D-proline. In some embodiments, provided is a co-crystal of (+) epicatechin:L-proline.

The co-crystal of epicatechin:aco-crystal former of Formula (I) may be present in various ratios. In some embodiments, the ratio is in the range of 1:3 to 3:1. The ratio can be a molar ratio or a weight ratio. In some embodiments, the co-crystal contains epicatechin:a co-crystal former of Formula (I) at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal contains epicatechin:aco-crystal former of Formula (I) at a ratio of about 1:3, about 1:2.5, about 1:2, about 1:1.5, about 1:1, about 1.5:1, about 2:1, about 2.5:1, or about 3:1. In some embodiments, the co-crystal contains epicatechin:aco-crystal former of Formula (I) at a ratio of about 1:1. In some embodiments, the co-crystal contains (+) epicatechin:aco-crystal former of Formula (I) at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal contains (+) epicatechin:a co-crystal former of Formula (I) at a ratio of about 1:3, about 1:2.5, about 1:2, about 1:1.5, about 1:1, about 1.5:1, about 2:1, about 2.5:1, or about 3:1. In some embodiments, the co-crystal contains (+) epicatechin:aco-crystal former of Formula (I) at a ratio of about 1:1. In some embodiments, the co-crystal contains (−) epicatechin:aco-crystal former of Formula (I) at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal contains (−) epicatechin:aco-crystal former of Formula (I) at a ratio of about 1:3, about 1:2.5, about 1:2, about 1:1.5, about 1:1, about 1.5:1, about 2:1, about 2.5:1, or about 3:1. In some embodiments, the co-crystal contains (−) epicatechin:aco-crystal former of Formula (I) at a ratio of about 1:1.

In some embodiments, the co-crystal contains epicatechin:trigonelline at a ratio of from about 1:3 to about 3:1, from about 1:2 to about 2:1, from about 1:3 to about 2:1, from about 1:2 to about 3:1, form about 3:1 to about 1:1, or from about 1:1 to about 1:3. In some embodiments, the co-crystal contains epicatechin:trigonelline at a ratio of about 1:3, about 1:2.5, about 1:2, about 1:1.5, about 1:1, about 1.5:1, about 2:1, about 2.5:1, or about 3:1. In some embodiments, the co-crystal contains epicatechin:trigonelline at a ratio of about 1:1. In some embodiments, the co-crystal contains epicatechin:proline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal contains epicatechin:proline at a ratio of about 1:3, about 1:2.5, about 1:2, about 1:1.5, about 1:1, about 1.5:1, about 2:1, about 2.5:1, or about 3:1. In some embodiments, the co-crystal contains epicatechin:proline at a ratio of about 1:1.

In some embodiments, the co-crystal contains (+) epicatechin:trigonelline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal contains (+) epicatechin:trigonelline at a ratio of about 1:3, about 1:2.5, about 1:2, about 1:1.5, about 1:1, about 1.5:1, about 2:1, about 2.5:1, or about 3:1. In some embodiments, the co-crystal contains (+) epicatechin:trigonelline at a ratio of about 1:1. In some embodiments, the co-crystal contains (−) epicatechin:trigonelline at a ratio of from about 1:3 to about 3:1, from about 1:2 to about 2:1, from about 1:3 to about 2:1, form about 1:2 to about 3:1, from about 3:1 to about 1:1, or from about 1:1 to about 1:3. In some embodiments, the co-crystal contains (−) epicatechin:trigonelline at a ratio of about 1:3, about 1:2.5, about 1:2, about 1:1.5, about 1:1, about 1.5:1, about 2:1, about 2.5:1, or about 3:1. In some embodiments, the co-crystal contains (−) epicatechin:trigonelline at a ratio of about 1:1.

In some embodiments, the co-crystal contains (+) epicatechin:D-proline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal contains (+) epicatechin:D-proline at a ratio of about 1:3, about 1:2.5, about 1:2, about 1:1.5, about 1:1, about 1.5:1, about 2:1, about 2.5:1, or about 3:1. In some embodiments, the co-crystal contains (+) epicatechin:D-proline at a ratio of about 1:1. In some embodiments, the co-crystal contains (−) epicatechin:L-proline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal contains (−) epicatechin:L-proline at a ratio of about 1:3, about 1:2.5, about 1:2, about 1:1.5, about 1:1, about 1.5:1, about 2:1, about 2.5:1, or about 3:1. In some embodiments, the co-crystal contains (−) epicatechin:L-proline at a ratio of about 1:1.

The present invention provides co-crystals that can be characterized by an X-ray diffraction pattern having characteristic peaks, in terms of 20. The relative intensities of the peaks can vary, depending upon the sample preparation technique, the sample mounting procedure and the particular instrument employed. Moreover, instrument variation and other factors can affect the 2-theta values. In some embodiments, the XRPD peak assignments can vary by plus or minus about 0.2°.

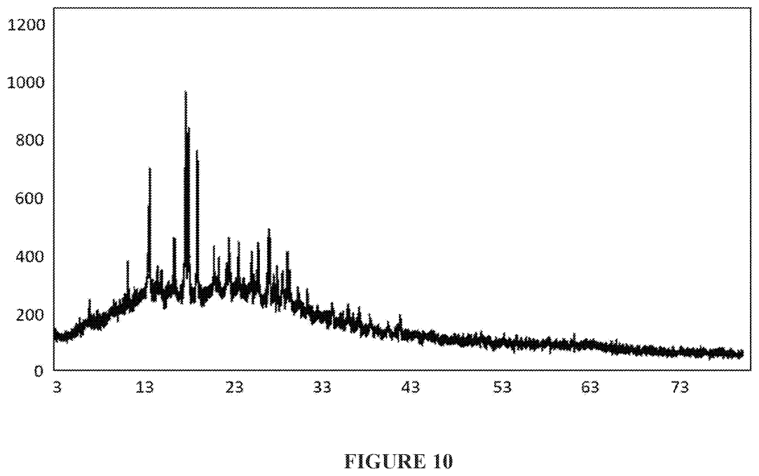

In some embodiments, a co-crystal of (−) epicatechin:trigonelline at a molar ratio of 1:1 is characterized by an X-Ray diffraction pattern comprising a peak, in terms of 20, at about 17.6°. In some embodiments, the X-Ray diffraction pattern further includes characteristic peaks, in terms of 20, at about 18.0°, about 19.0° and/or about 13.6°. In some embodiments, the X-Ray diffraction pattern further includes characteristic peaks, in terms of 2θ, at about 27.0°, about 16.4°, about 20.9°, about 22.5°, about 23.6°, about 25.0°, about 25.7°, and/or about 29.0°. In some embodiments, the co-crystal of (−) epicatechin:trigonelline at a molar ratio of 1:1 is characterized by an X-Ray diffraction pattern substantially as shown in FIG. 10.

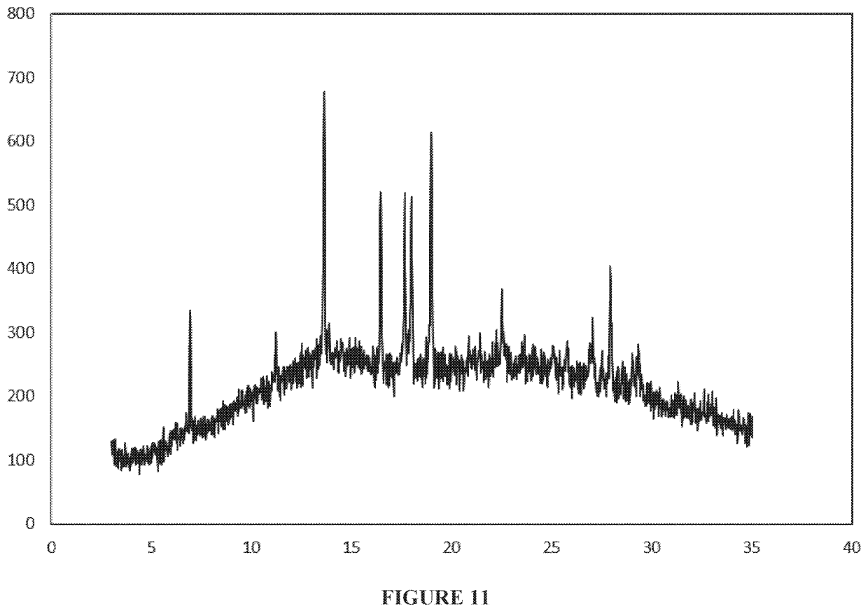

In some embodiments, a co-crystal of (+) epicatechin:trigonelline at a molar ratio of 1:1 is characterized by an X-Ray diffraction pattern comprising a peak, in terms of 20, at about 13.6°. In some embodiments, the X-Ray diffraction pattern further includes a characteristic peak, in terms of 20, at about 19.0°. In some embodiments, the X-Ray diffraction pattern further includes characteristic peaks, in terms of 20, at about 6.9°, about 16.4°, about 17.6°, about 18.0°, about 22.5°, and/or about 27.9°. In some embodiments, the co-crystal of (+) epicatechin:trigonelline at a molar ratio of 1:1 is characterized by an X-Ray diffraction pattern substantially as shown in FIG. 11.

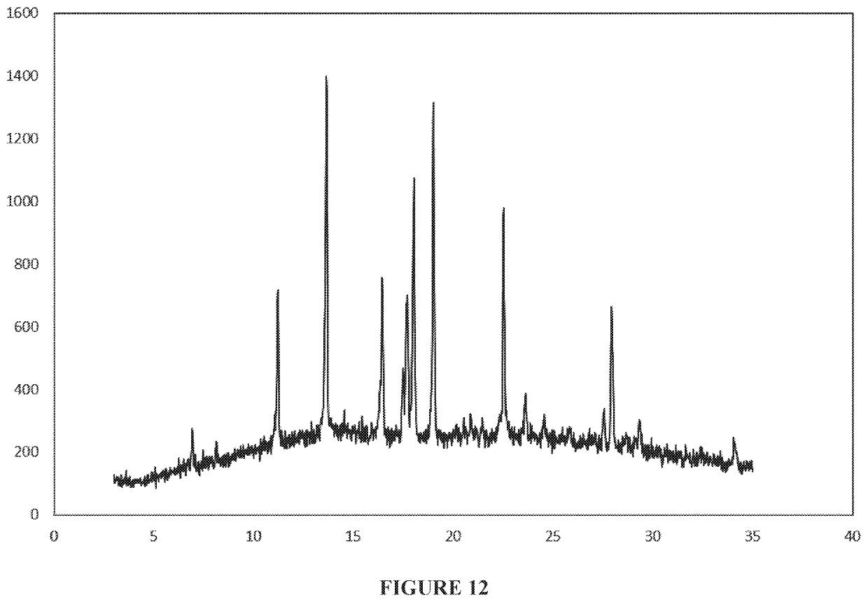

In some embodiments, a co-crystal of (+) epicatechin:trigonelline at a molar ratio of 1:2 is characterized by an X-Ray diffraction pattern comprising a peak, in terms of 20, at about 13.6°. In some embodiments, the X-Ray diffraction pattern further includes characteristic peaks, in terms of 20, at about 19.0° and/or about 18.0°. In some embodiments, the X-Ray diffraction pattern further includes characteristic peaks, in terms of 20, at about 11.2°, about 16.4°, about 17.7°, about 22.5°, and/or about 27.9°. In some embodiments, the co-crystal of (+) epicatechin:trigonelline at a molar ratio of 1:2 is characterized by an X-Ray diffraction pattern substantially as shown in FIG. 12.

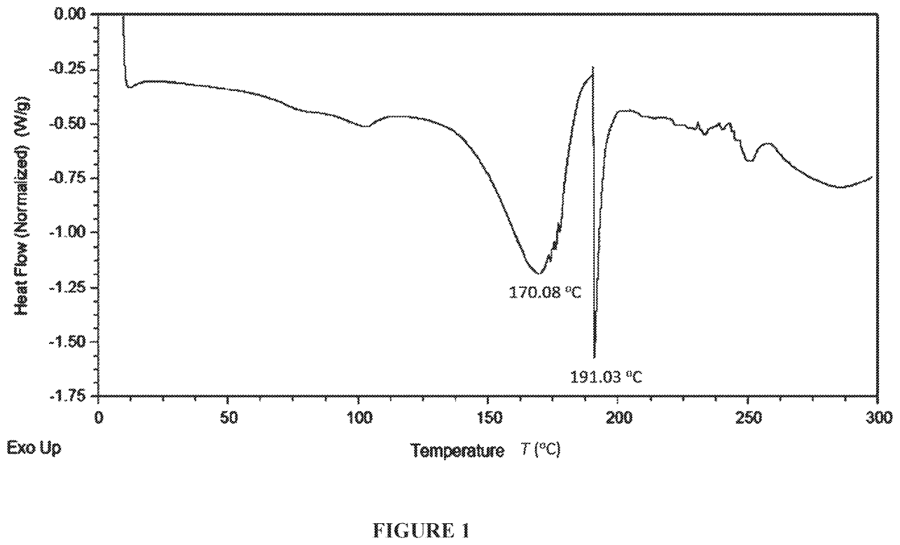

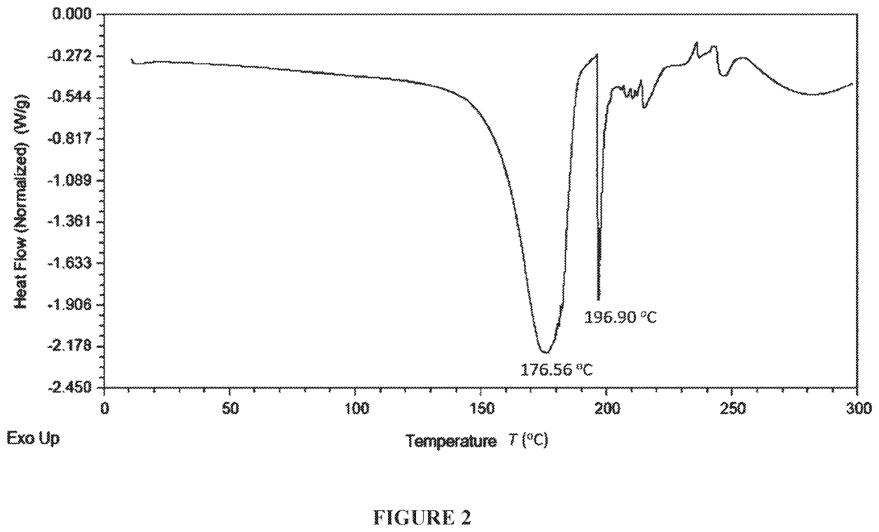

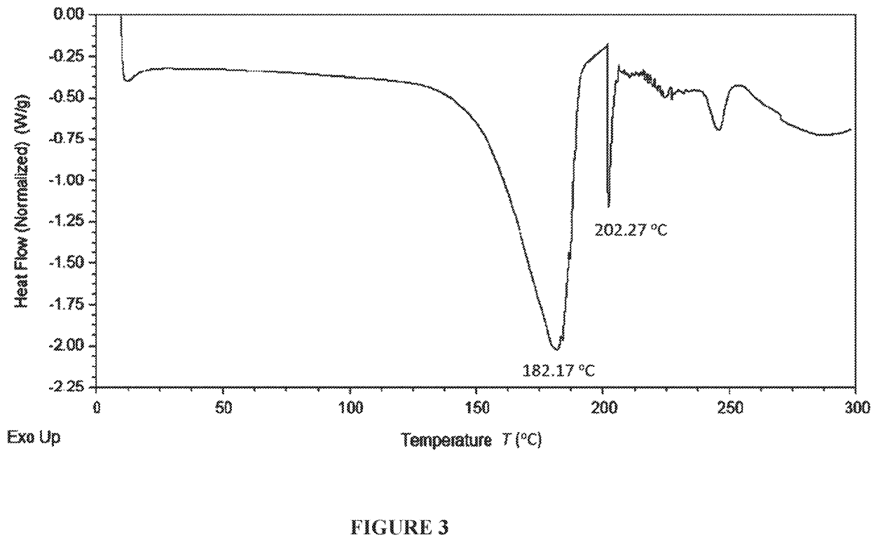

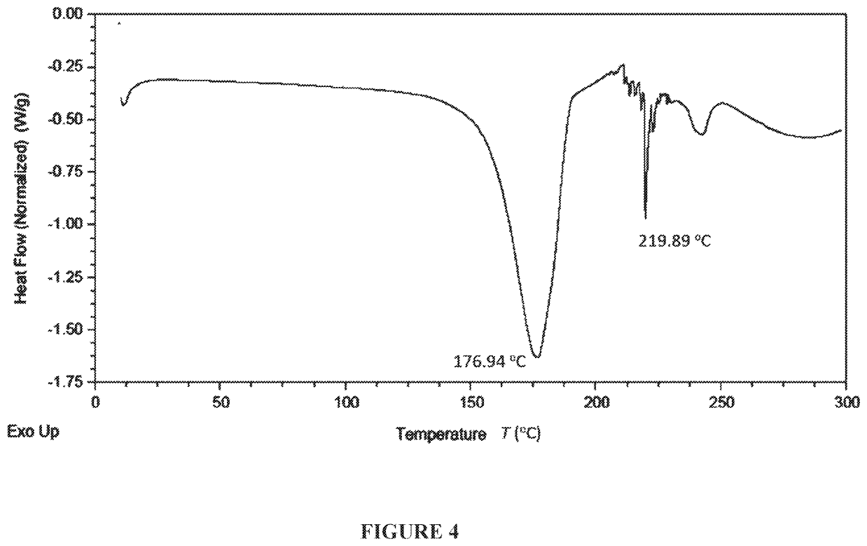

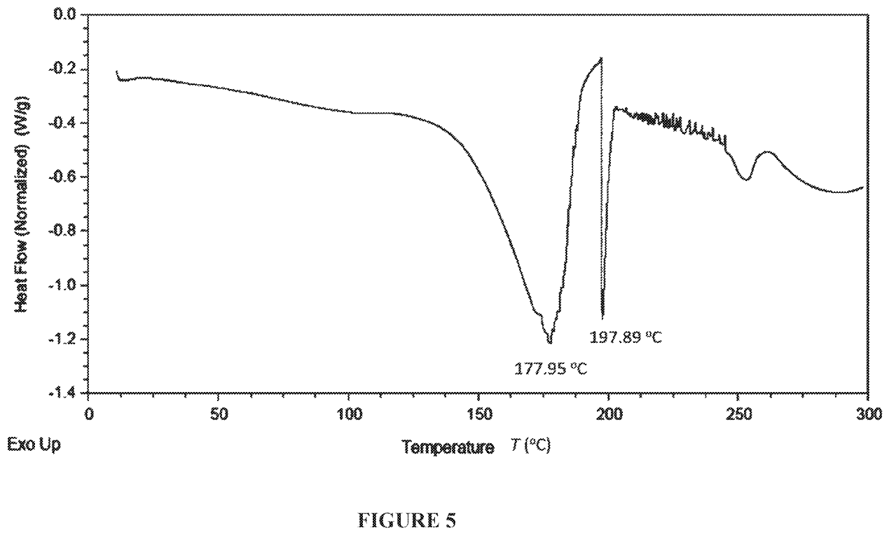

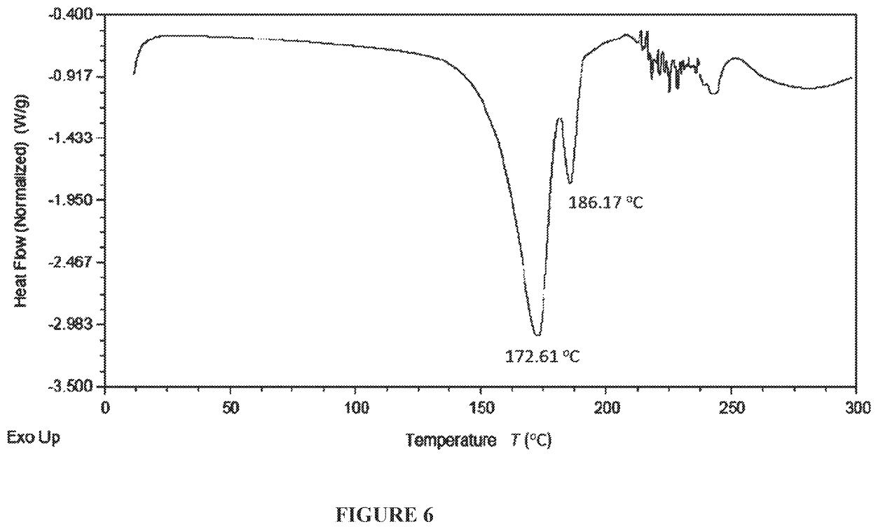

The co-crystals can also be identified by its characteristic differential scanning calorimetry (DSC) trace. In some embodiments, the co-crystals provided herein have characteristic differential scanning calorimetry (DSC) patterns substantially as shown in FIGS. 1-6, 15, 16, 18 and 19.

In some embodiments, a co-crystal of (−) epicatechin:trigonelline at a molar ratio of 1:1 is characterized by a melting point ranging from about 169 to about 175° C. In some embodiments, a co-crystal of (+) epicatechin:trigonelline at a molar ratio of 1:1 is characterized by a meting point ranging from about 165 to about 178° C. or from about 165 to about 169° C. In some embodiments, a co-crystal of (+) epicatechin:trigonelline at a molar ratio of 1:2 is characterized by a melting point ranging from about 172 to about 185° C. In some embodiments, a co-crystal of (+) epicatechin:(D) proline at a molar ratio of 1:1 is characterized by a melting point ranging from about 198 to about 202° C. In some embodiments, a co-crystal of (−) epicatechin:(L) proline at a molar ratio of 1:1 is characterized by a melting point ranging from about 195 to about 198° C.

### Compositions

In another embodiment, the present invention provides pharmaceutical compositions comprising co-crystals of epicatechin:aco-crystal former of Formula (I) together with one or more pharmaceutically acceptable excipients. In some embodiments, the composition comprises one co-crystalline form of epicatechin:aco-crystal former of Formula (I). In some embodiments, the composition comprises two or more co-crystalline forms of epicatechin:a co-crystal former of Formula (I). For example, the pharmaceutical composition in the present invention can contain co-crystals of (+)/(−) epicatechin:trigonelline, co-crystals of (+)/(−) epicatechin:proline, or any combination thereof.

In some embodiments, provided is a pharmaceutical composition comprising a co-crystal of (+) epicatechin:trigonelline. In some embodiments, provided is a pharmaceutical composition comprising a co-crystal of (−) epicatechin:trigonelline. In some embodiments, provided is a pharmaceutical composition comprising a co-crystal of (+) epicatechin:D-proline. In some embodiments, provided is a pharmaceutical composition comprising a co-crystal of (+) epicatechin:L-proline.

The pharmaceutical composition comprising a co-crystal of epicatechin:aco-crystal former of Formula (I) present in various ratios. In some embodiments, the ratio is in the range of 1:3 to 3:1. The ratio can be a molar ratio or a weight ratio. In some embodiments, the co-crystal in the pharmaceutical composition contains epicatechin:aco-crystal former of Formula (I) at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal in the pharmaceutical composition contains epicatechin:aco-crystal former of Formula (I) at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains epicatechin:aco-crystal former of Formula (I) at a ratio of about 1:1. In some embodiments, the co-crystal in the pharmaceutical composition contains (+) epicatechin:aco-crystal former of Formula (I) at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal in the pharmaceutical composition contains (+) epicatechin:aco-crystal former of Formula (I) at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains (+) epicatechin:a co-crystal former of Formula (I) at a ratio of about 1:1. In some embodiments, the co-crystal in the pharmaceutical composition contains (−) epicatechin:aco-crystal former of Formula (I) at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal in the pharmaceutical composition contains (−) epicatechin:aco-crystal former of Formula (I) at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains (−) epicatechin:a co-crystal former of Formula (I) at a ratio of about 1:1.

In some embodiments, the co-crystal in the pharmaceutical composition contains epicatechin:trigonelline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal contains epicatechin; trigonelline at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains epicatechin:trigonelline at a ratio of about 1:1. In some embodiments, the co-crystal in the pharmaceutical composition contains epicatechin:proline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal in the pharmaceutical composition contains epicatechin:proline at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains epicatechin:proline at a ratio of about 1:1.

In some embodiments, the co-crystal in the pharmaceutical composition contains (+) epicatechin:trigonelline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal in the pharmaceutical composition contains (+) epicatechin:trigonelline at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains (+) epicatechin:trigonelline at a ratio of about 1:1. In some embodiments, the co-crystal in the pharmaceutical composition contains (−) epicatechin:trigonelline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal in the pharmaceutical composition contains (−) epicatechin:trigonelline at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains (−) epicatechin:trigonelline at a ratio of about 1:1.

In some embodiments, the co-crystal in the pharmaceutical composition contains (+) epicatechin:D-proline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal in the pharmaceutical composition contains (+) epicatechin:D-proline at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains (+) epicatechin:D-proline at a ratio of about 1:1. In some embodiments, the co-crystal in the pharmaceutical composition contains (−) epicatechin:L-proline at a ratio of from about 1:3 to about 3:1, from about 1:2 to about 2:1, from about 1:3 to about 2:1, from about 1:2 to about 3:1, from about 3:1 to about 1:1, or from about 1:1 to about 1:3. In some embodiments, the co-crystal in the pharmaceutical composition contains (−) epicatechin:L-proline at a ratio of about 1:3, about 1:2.5, about 1:2, about 1:1.5, about 1:1, about 1.5:1, about 2:1, about 2.5:1, or about 3:1. In some embodiments, the co-crystal in the pharmaceutical composition contains (−) epicatechin:L-proline at a ratio of about 1:1.

### Methods of Preparation

In another aspect, the present invention provides a process for preparing a novel co-crystal of epicatechin:aco-crystal former of Formula (I). In some embodiments, the process comprises the following steps:

(i) dissolving epicatechin and a co-crystal former of Formula (I) in a solvent to obtain a solution;

(ii) heating the solution obtained from step (i);

(iii) resting the heated solution of step (ii); and

(iv) obtaining the co-crystals.

In some embodiments of any of the methods of preparing a co-crystal provided herein, the co-crystal former of Formula (I) is added to the solvent in a neutral form, including a zwitter-ionic form. In some embodiments of any of the methods of preparing a co-crystal provided herein, the co-crystal former of Formula (I) is added to the solvent in a positively charged (e.g., salt) form. In some embodiments of any of the methods of preparing a co-crystal provided herein, the co-crystal former of Formula (I) is added to the solvent in a negatively charged (e.g., salt) form. In some. In some embodiments, an epicatechin:trigonelline co-crystal is prepared using trigonelline hydrochloride.

Epicatechin, including (+) epicatechin and (−) epicatechin, can be prepared using methods including, without limitation, as described in WO2012/101652 and WO2014/115174, which are incorporated by reference herein in their entirety.

The solvent in the process of the present invention may be an organic solvent or an aqueous solvent or mixtures thereof. The solvent may preferably be selected from the group consisting of water; alcohols such as methanol, ethanol, 1-propanol, 2-propanol (isopropanol), 1-butanol, 2-butanol, t-butyl alcohol, 1-pentanol, 2-pentanol, 2-ethoxyethanol, ethylene glycol, glycerol, and the like: and any mixtures thereof, preferably the solvent is ethanol or 2-propanol. Aqueous alcoholic solutions are preferred. In some embodiments, an organic solution or an aqueous solution comprises two or more solvents, for example, water and ethanol or water and isopropanol. In some embodiments, two solvents may be present in a molar or weight ratio that varies in the range of 1:100 to 100:1, 1:10 to 10:1, or 1:3 to 3:1. In some embodiments, two solvents may be present in a molar or weight ratio of about 1:100, about 1:50, about 1:20, about 1:10, about 1:5, about 1:3, about 1:2, about 1:1, about 2:1, about 3:1, about 5:1, about 10:1, about 20:1, about 50:1, or about 100:1. In some embodiments, the solvent is ethanol:water (1:1). In some embodiments, the solvent is isopropanol.

The solution may be heated at a temperature between 50 to 60° C., preferably at temperature between 55 to 58° C. until a clear solution is obtained. In some embodiments, the solution is heated at a temperature of from about 40 to about 100, from about 50 to about 80, from about 60 to about 70, from about 65 to about 75, or from about 70 to about 80° C. In some embodiments, the solution is heated at a temperature of from about 65 to about 75, such as about 70° C.

The solution may be rested for a period of 1 hour to 7 days (e.g., 1-7 days) at a temperature lower than the heated temperature (e.g., in a range of 25-37° C.). The temperature can range from 0-40° C., for example, at about 4° C., at about 20° C., at about 25° C., or at about 37° C. In some embodiments, the solution is rested for a period of about 1 day, about 2 days, about 3 days, about 4 days, about 5 days, about 6 days, or about 7 days. In some embodiments, the solution is rested for a period of about 2 hours, about 12 hours, about 16 hours, about 18 hours, about 20 hours, about 24 hours, about 36 hours, about 48 hours, about 60 hours, about 72 hours, or about 90 hours. In some embodiments, resting the heated solution includes cooling the solution below mom temperature, for example at refrigeration temperature (e.g., 4° C.). The solution may be cooled to 4° C. for a period of about 1, about 2, about 3, about 4, about 5, about 6, or about 7 days. In some embodiments, the solution is cooled to 4° C. for a period of about 12, about 24, about 36, about 48, about 60, about 72, or about 90 hours. In some embodiments, the solution is rested for a period of about 1, about 2, about 3, about 4, about 5, about 6, or about 7 days at a temperature in the range of about 0 to about 40, about 10 to about 30, about 0 to about 10, about 10 to about 20, about 20 to about 30, or about 30 to about 40° C. In some embodiments, the solution is rested for a period of about 24 hours at 15-20° C. (e.g., 18° C.). In some embodiments, the solution is rested for a period of about 48 hours at 15-20° C. (e.g., 18° C.). In some embodiments, the solution is rested for a period of about 16 hours at 2-6° C. (e.g., 4° C.). The resting can be conducted in one or more steps, each at a different temperature for a certain period of time. In some embodiments, the solution is kept at room temperature for 2 h and then kept at 4° C. (refrigerator) for 16 h.

In some embodiments, the yield of co-crystal is at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

The co-crystal formed may be evaluated for its physicochemical parameters using methods known in the art, including through analytical techniques such as infrared (IR) spectroscopy, X-ray powder diffraction (XRPD, also referred to as PXRD), differential scanning calorimetry (DSC), and the like.

### Pharmacokinetics and Pharmacodynamics

The co-crystals described herein may exhibit advantageous properties, for instance, in comparison to (+) or (−) epicatechin not in a co-crystalline form. The co-crystals of the present invention improve the pharmacokinetic profile of epicatechin, both in terms of Cmax and AUC. The co-crystals of the present invention decrease the number of doses needed to achieve a desired effect, and/or create a more effective and/or a safer drug of epicatechin. The co-crystals have pharmacokinetic and pharmacodynamic advantages. The co-crystals provided herein may have improvements compared to epicatechin not in a co-crystalline form with respect to any one of more of the following properties: solubility is increased, bioavailability is increased, stability is increased, dose-response is increased, pharmacokinetic profile (e.g., Cmax, AUC) is improved; and inter-subject variability is reduced.

Pharmacokinetic (PK) and pharmacodynamic (PD) properties of the co-crystals can be assessed using methods known in the art. For example, PK and/or PD properties may be evaluated in animal models such as SD rats. The co-crystal may be dosed in suitable vehicle, such as a vehicle in which it retains its co-crystalline form. Vehicles that may be used for PK and/or PD analysis of the co-crystals described herein include, without limitation, carboxymethylcellulose (CMC) and Tween 80.

In some embodiments, the co-crystal of epicatechin (e.g., (+) epicatechin, (−) epicatechin) has a Cmax that is at least 10%, 20%, 30%, 40%, 50%, 60%, 70%, 80%, 90%, 100%, 125%, 150%, 175%, 200%, 250%, 300%, 350%, or 400% greater than the Cmax for epicatechin (e.g., (+) epicatechin, (−) epicatechin) not in a co-crystalline form. In some embodiments, the co-crystal of epicatechin (e.g., (+) epicatechin, (−) epicatechin) has a Cmax of at least about 400, about 500, about 600, about 700, about 800, about 900, or about 1000 nM. In some embodiments, the co-crystal of epicatechin (e.g., (+) epicatechin, (−) epicatechin) has an AUC that is at least 10%, 20%, 30%, 40%, 50%, 60%, 70%, 80%, 90%, 100%, 125%, 150%, 175%, 200%, 250%, 300%, 350%, or 400% greater than the AUC for epicatechin (e.g., (+) epicatechin, (−) epicatechin) not in a co-crystalline form. In some embodiments, the co-crystal of epicatechin (e.g., (+) epicatechin, (−) epicatechin) has an AUC of at least about 700, about 800, about 900, about 1000, about 1100, about 1200, about 1300, about 1400, about 1500, about 1600, about 1700, about 1800, about 1900, or about 2000 nM.

### Methods of Use

The co-crystals of the present invention are used to improve the physicochemical properties of pharmaceutical and nutraceutical ingredients.

The co-crystals of the present invention may be used for all indications in which epicatechin is indicated, including, without limitation, any of the disease or conditions described in WO2012/170430, WO2013/022846, WO2013/142816, US2018/0193306, WO2014/162320, WO2017/221269, and WO2018/083713, each of which is hereby incorporated by reference in its entirety.

In yet another aspect, the present invention provides methods for treating diseases or disorders that would benefit from increased expression of Electron transfer Chain (ETC), particularly ETC IV. The methods involve administering to a subject in need thereof a therapeutically effective amount of the co-crystals and the pharmaceutical compositions of the present invention.

The vast majority of the body's need for ATP is supplied through the process of oxidative phosphorylation, carried out in the mitochondria in all tissues. There are 5 protein complexes, known as the Electron Transport Complexes that effect ATP synthesis. ETC I, II, III, and IV mediate electron transport. ETC I, III, and IV also function as proton pumps that maintain an electrochemical gradient necessary for activity of ETC V, the ATP synthase enzyme that makes ATP from ADP. Complex r, also known as cytochrome c oxidase, (COX), consists of 14 subunits whose assembly into a functional complex requires an additional 30 protein factors. ETC IV is particularly important to oxidative phosphorylation. It is the only one of the ETC complexes to manifest tissue-specific and developmentally regulated isoforms, allowing precise regulation of oxidative phosphorylation under a variety of metabolic demands. Thus the ETC IV (COX) protein complex is considered to be the rate-limiting step in oxidative phosphorylation. Small positive or negative changes in ETC IV can exert a significant impact on health. Selective activation of COX activity has been associated with improved cognition, improved neuronal cell survival under stress, and improved wound healing. Mutations in the numerous proteins that comprise or regulate the activity of ETC IV reveal the pathological consequences of even modest decreases in ETC IV activity. As little as a 30% reduction in COX activity has been shown to induce cardiomyopathy or be associated with the development of neurodegenerative diseases such as Alzheimers. Decreases in COX (ETCIV) expression due to mutations or molecular manipulation have been associated with loss of muscle endurance and speed, muscle dystonia, immunodeficiency states due to impaired T cell maturation, cardiomyopathy, particularly of the aging phenotype, ataxia, neurodegeneration, increased toxicity in the setting of ischemia, pulmonary inflammation and fibrosis, encephalopathy, vascular insufficiency, and stimulation of cancer cell proliferation. Additional specific diseases associated with COX subunit isoform mutations causing loss of function include exocrine pancreatic insufficiency, inflammatory lung disease, Charcot-Marie-Tooth disease, infantile encephalomyopathy, and Leigh syndrome neurodegeneration with epilepsy.

The following conditions associated with loss of COX expression or function would be expected to be therapeutically responsive to a potent, preferential inducer of COX (ETC IV) expression: impaired cognition, neurodegenerative diseases such as Alzheimer's or Leigh syndrome, dystonia, sarcopenia, cardiomyopathy of aging or other diseases associated with mitochondrial dysfunction, ischemic vascular disease, immunodeficiency states, ataxia, pulmonary inflammation and fibrosis, infantile encephalomyopathy, epilepsy, Charcot-Marie-Tooth disease, exocrine pancreatic insufficiency, impaired wound healing, growth of cancer cells.

In some embodiments, the co-crystals and pharmaceutical compositions provided herein may be used for inducing mitochondrial biogenesis, including biogenesis of any one or more of ETC I, II, III, IV, and V.

In addition, epicatechin can be used in lowering the elevated triglycerides so a co-crystal containing epicatechin would be use in medicament for conditions associated with elevated triglycerides, such as metabolic syndrome, Type II diabetes, congenital hyperlipidemias, and drug-induced hyperlipidemia, as is observed with corticosteroid treatments.

In another embodiment, provided are methods for prophylactic and/or therapeutic treatment of conditions related to mitochondrial dysfunction resulting from administration of one or more chemical compositions that exhibit mitochondrial toxicity. In some embodiments, the mitochondrial toxicity is identified based on or associated with one or more biological effects, which include, but are not limited to, abnormal mitochondrial respiration, abnormal oxygen consumption, abnormal extracellular acidification rate, abnormal mitochondrial number, abnormal lactate accumulation, and abnormal ATP levels. In some embodiments, the mitochondrial toxicity is identified based on or associated with one or more physiological manifestations, which include, but are not limited to, elevations in markers known to relate to injury to the heart, liver, and/or kidney, elevated serum liver enzymes, elevated cardiac enzymes, lactic acidosis, elevated blood glucose, and elevated serum creatinine. In another embodiment, provided are methods for treating chronic mitochondrial depletion and the symptoms arising as a result of drug-associated toxicity or as a combination of drug associated toxicity occurring within a background of biological depletion of mitochondrial number, as occurs in diabetes, obesity, and during the course of aging. In another embodiment, provided are methods for treating chronic perturbation of mitochondria function or structure, including chronic myopathy, sarcopenia, persistent diabetes, chronic fatigue syndromes, gastrointestinal symptoms, liver, and cardiovascular dysfunction and failure, neurological symptoms, impaired sleep, and persistent alteration in cognitive acuity or function, such as memory.

In another embodiment, provided are methods for treating, preventing, or reversing injury to skeletal or cardiac muscles, for treating or preventing diseases relating to the structure and function of skeletal or cardiac muscles, and for inducing regeneration or restructuring of skeletal or cardiac muscle as a means for treating disease relating to abnormalities in the skeletal or cardiac muscle structure and function in a subject.

In some embodiments, provided are methods for treatment of impaired skeletal or cardiac muscle function due to aging, obesity, disuse or inactivity, exposure to potentially toxic nutritional agents such as fructose, or exposure to inadequate nutrition such as starvation or malnutrition.

In some embodiments, provided are methods for the treatment of muscle-related side effects of athletic training or competition including soreness, cramping, weakness, pain, or injury.

In some embodiments, provided are methods for the treatment of skeletal or cardiac muscle diseases associated with ischemia, or impaired or inadequate blood flow. In some embodiments, the diseases are selected from the group consisting of atherosclerosis, trauma, diabetes, vascular stenosis, peripheral arterial disease, vasculopathy, and vasculitis.

In some embodiments, provided are methods for the treatment of diseases associated with genetic disorders that directly or indirectly affect the number, structure, or function of cardiac muscle cells or skeletal muscle cells. In some embodiments, the disease is selected from the group consisting of muscular dystrophies and Friedreich's ataxia.

In some embodiments, provided are methods for the treatment of diseases associated with impaired neurological control of muscular activity resulting in consequent abnormalities in structure and function of skeletal muscles due to inactivity, aberrant contractility, or contracted states. In some embodiments, the disease is selected from the group consisting of peripheral denervation syndromes, trauma, amyotrophic lateral sclerosis, meningitis, and structural abnormalities of the spine, whether congenital or acquired.

In some embodiments, provided are methods for the treatment of diseases associated with loss of number, loss of function, or loss of correct, optimally efficient internal organization of skeletal muscle cells or cardiac muscle cells. In some embodiments, the disease is muscle wasting. In some embodiments, the disease is sarcopenia. In some embodiments, sarcopenia is associated with a variety of disorders, including aging, diabetes, abnormal metabolic conditions, infection, inflammation, autoimmune disease, cardiac dysfunction, arthritis congestive heart failure, aging, myocarditis, myositis, polymyalgia rheumatica, polymyositis, HIV, cancer, side effects of chemotherapy, malnutrition, aging, inborn errors of metabolism, trauma, stroke, and neurological impairment.

In some embodiments, the method of treating diseases associated with loss of number, loss of function, or loss of correct, optimally efficient internal organization of skeletal muscle cells or cardiac muscle cells further comprises exercise or programmatic sequences or intensities of exercise.

In some embodiments, provided are methods for enhancing sports performance, endurance, building muscle shape or strength, or facilitating recovery from the effects of training or competition.

In some embodiments, provided are methods for treating muscle injury, weakness, or pain associated with the administration of medicines. In some embodiments, provided are methods for use to prevent, ameliorate, or reverse muscle injury associated with medicines that damage mitochondria and/or cause myopathy as a secondary consequence.

In some embodiments of any one of the embodiments disclosed above, the skeletal or cardiac muscle injury of dysfunction in the subject is identified based on or associated with one or more physiological manifestations, which include, but are not limited to, elevated plasma levels of cardiac or skeletal muscle enzymes or proteins, such as myoglobin, troponin, or creatine phosphokinase, lactic acidosis, and elevated serum creatinine.

In some embodiments, provided are methods for stimulating the increased number or function of skeletal muscle cells or contractile muscle cells. Such stimulation of muscle cells may comprise stimulation of one or more aspects of muscle cell function, including cell division, muscle cell regeneration, activation of muscle satellite cells and their differentiation into adult muscle cells, recovery from injury, increased number or function of mitochondria or processes serving mitochondrial function, increased expression of proteins contributing to contractility, regulation of biochemical or translational processes, mitoses, or transduction of mechanical energy via dystrophin or other attachment processes. The methods and compositions described herein can assist in prevention of the consequences of muscle injury or dysfunction which have not yet occurred, as well as provide for the active therapy of muscle injury, dysfunction, or diseases which have already occurred.

In some embodiments, provided are methods of using muscle proteins whose expression is stimulated by administration of a co-crystal or pharmaceutical composition provided herein as diagnostic biomarkers by which to determine the time and degree of muscle response to the therapeutic methods and compositions disclosed herein. Such biomarkers may be determined by measuring in tissue, plasma, blood, or urine the proteins themselves or the DNA or RNA nucleotides that encode for the proteins. In one embodiment, a decrease in the body of useful muscle proteins, such as dystrophin, or the presence of inhibitory proteins, such as thromobospondin, may be used to diagnose the severity of the abnormality of cardiac muscle structure or function or the probability of response to the therapeutic methods and compositions described herein. In another embodiment, changes in the levels of such biomarkers may be used to gauge the success or failure of certain therapeutic modalities, including those disclosed herein, in order to optimize the dose and to decide whether to maintain or change therapeutic methods and compositions.

In some embodiments, provided are methods of inducing follistatin production, inhibiting myostatin production, and/or increasing the ratio of follistatin to myostatin. This may be, for example, in associated with treating a muscle or bone condition or disorder.

Additional embodiments disclosed herein relate to a method to induce the increased cellular or muscular or bodily production of follistatin and follistatin-like proteins in order to reverse or ameliorate weakness of bone, thus preventing bone fractures, which may in some instances be caused by administration of compounds known to induce weakness of or damage to bone, impairment of bone generation, or impairment of bone growth, including but not limited to corticosteroids such as prednisone or deflazacort, anticonvulsants such as phenytoin and phenobarbital, chemotherapeutics such as aromatase inhibitors, and progestins. Further methods relate to inducing the increased cellular or muscular or bodily production of follistatin or follistatin-like proteins in order to reverse or ameliorate weakness of bone strength, thus preventing bone fractures, which may in some instances be associated with genetic predisposition, aging, inactive lifestyle, or low estrogen states such as menopause or post oophorectomy; a method to induce the increased cellular or muscular or bodily production of follistatin or follistatin-like proteins in order to reverse or ameliorate weakness of bone caused by medical conditions known to be associated with weakness of, or damage to, bone, impairment of bone generation, or impairment of bone growth, such celiac disease, kidney or liver disease, and immunomodulatory diseases such as systemic lupus erythematosus and rheumatoid arthritis, a method to induce the increased cellular or muscular or bodily production of follistatin or follistatin-like proteins in order to reverse or ameliorate weakness of bone in conjunction with the administration of other agents used to treat osteoporosis including calcium, Vitamin D, and calcitonin, in order to prevent bone fractures; a method to method to induce increased cellular or muscular or bodily production of follistatin or follistatin-like proteins as a therapeutic to accelerate the healing of bone fractures or to increase the degree of recovery from a bone fracture, such as those experienced in accidents, athletics, or combat; and a method to induce increased cellular or muscular or bodily production of follistatin or follistatin-like proteins in order to prevent systemic loss of bone density, and thus prevent subsequent bone fractures, during the recovery period after orthopedic surgery or after the onset of a disease or condition necessitating long periods of bed rest or physical inactivity, which are known to result in decreased bone density and muscle weakness.

In some embodiments, provided are methods for treating or preventing neurodegenerative diseases, such as amyotrophic lateral sclerosis (ALS), Parkinson's disease, Huntington's disease, spinal cord injury or abnormality, and peripheral and central neuropathies.

In some embodiments, provided arc methods for treating or preventing celiac disease, kidney disease, liver disease, inflammatory diseases such as systemic lupus erythematosus and rheumatoid arthritis, osteoporosis, and bone fracture.

Conditions that may be treated by the co-crystals, pharmaceutical compositions, and methods provided herein include: impaired skeletal and cardiac muscle function, recovery of skeletal or cardiac muscle health or function, functionally significant regeneration of skeletal or cardiac muscle cells or function.

In some embodiments, provided are methods for treating acute coronary syndromes, including but not limited to myocardial infarction and angina; acute ischemic events in other organs and tissues, renal injury, renal ischemia and diseases of the aorta and its branches; injuries arising from medical interventions, including but not limited to coronary artery bypass grafting (CABG) procedures and aneurysm repair; cancer; and metabolic diseases, diabetes mellitus and other such disorders.

In some embodiments, provided are methods for treating or preventing dystrophinopathy, such as Duchenne muscular dystrophy. Becker muscular dystrophy, and DMD-associated cardiomyopathy.

In some embodiments, provided are methods for treating or preventing sarcoglycanopathy, including α-sarcoglycanopathy (LGMD2D), β-sarcoglycanopathy (LGMD2E), γ-sarcoglycanopathy (LGMD2C), S-sarcoglycanopathy (LGMD2F) and s-sarcoglycanopathy (myoclonic dystonia). Sarcoglycanopathies include four subtypes of autosomal recessive limb-girdle muscular dystrophy (LGMD2C, LGMD2D, LGMD2E, and LGMD2F) that are caused, respectively, by mutations in the SGCG, SGCA, SGCB, and SGCD genes.

In some embodiments, provided are methods for treating or preventing dysferlinopathy, such as Miyoshi myopathy, scapuloperoneal syndrome, distal myopathy with anterior tibial onset, and elevated level of muscular enzyme CK.

Provided is a method of treating or preventing any of the diseases or conditions described herein in a subject in need thereof, comprising administering to the subject a therapeutically effective amount of a co-crystal or pharmaceutical composition provided herein. Also provided is a co-crystal provided herein for use the manufacture of a medicament for treating or preventing any of the diseases or conditions described herein in a subject in need thereof. Also provided is a co-crystal or pharmaceutical composition provided herein for use in treating or preventing a disease or condition described herein in a subject in need thereof. Also provided is a co-crystal or pharmaceutical composition provided herein for use in medical therapy. Also provided is use of a co-crystal or pharmaceutical composition provided herein for treating or preventing a disease or condition described herein in a subject in need thereof.

### Dosages

The co-crystals and compositions disclosed and/or described herein are administered at a therapeutically effective dosage, e.g., a dosage sufficient to provide treatment for the disease state. While human dosage levels have yet to be optimized for the chemical entities described herein, generally, a daily dose ranges from about 0.01 to 100 mg/kg of body weight; in some embodiments, from about 0.05 to 10.0 mg/kg of body weight, and in some embodiments, from about 0.10 to 1.4 mg/kg of body weight. Thus, for administration to a 70 kg person, in some embodiments, the dosage range would be about from 0.7 to 7000 mg per day; in some embodiments, about from 3.5 to 700.0 mg per day, and in some embodiments, about from 7 to 100.0 mg per day. The amount of the chemical entity administered will be dependent, for example, on the subject and disease state being treated, the severity of the affliction, the manner and schedule of administration and the judgment of the prescribing physician. For example, an exemplary dosage range for oral administration is from about 5 mg to about 500 mg per day, and an exemplary intravenous administration dosage is from about 5 mg to about 500 mg per day, each depending upon the pharmacokinetics.

A daily dose is the total amount administered in a day. A daily dose may be, but is not limited to be, administered each day, every other day, each week, every 2 weeks, every month, or at a varied interval. In some embodiments, the daily dose is administered for a period ranging from a single day to the life of the subject. In some embodiments, the daily dose is administered once a day. In some embodiments, the daily dose is administered in multiple divided doses, such as in 2, 3, or 4 divided doses. In some embodiments, the daily dose is administered in 2 divided doses.

Administration of the co-crystals and compositions described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the co-crystal or composition is administered orally or intravenously. In some embodiments, the co-crystal or composition disclosed and/or described herein is administered orally.

Pharmaceutically acceptable compositions include solid, semi-solid, liquid and aerosol dosage forms, such as tablet, capsule, powder, liquid, suspension, suppository, and aerosol forms. The co-crystals disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

The co-crystals described herein can be administered either alone or in combination with one or more conventional pharmaceutical carriers or excipients (e.g., mannitol, lactose, starch, magnesium stearate, sodium saccharine, talcum, cellulose, sodium crosscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pa.

In some embodiments, the compositions will take the form of a pill or tablet and thus the composition may contain, along with a co-crystal disclosed and/or described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

Liquid pharmaceutically administrable compositions can, for example, be prepared by dissolving, dispersing or suspending etc. a co-crystal disclosed and/or described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the co-crystal contained in such parenteral compositions depends, for example, on the physical nature of the co-crystal, the activity of the co-crystal, and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a co-crystal disclosed and/or described herein in solution.

Pharmaceutical compositions of the co-crystal and compositions described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns.

In addition, pharmaceutical compositions can include a co-crystal disclosed and/or described herein and one or more additional medicinal agents, pharmaceutical agents, adjuvants, and the like.

### Kits

Also provided are articles of manufacture and kits containing any of the co-crystals or compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing a co-crystal or composition described herein and instructions for use. The kits may contain instructions for use in the treatment of a any disease provided herein in a subject in need thereof. A kit may additionally contain any materials or equipment that may be used in the administration of the co-crystal or composition, such as vials, syringes, or IV bags. A kit may also contain sterile packaging.

Illustrative examples of certain analytical data for the co-crystals of epicatechin:trigonelline and in co-crystals of epicatechin:proline obtained in the examples are set forth in the Figures provided herewith (including FIGS. 1-3).

Certain specific aspects and embodiments of the present invention will be explained in more detail with reference to the following examples, which are provided only for purposes of illustration and should not be construed as limiting the scope of the invention in any manner.

The following Examples will further illustrate the present invention, which by no means limit the scope of the invention.

### EXAMPLES

### Example 1. Preparation of Epicatechin_Trigonelline Cocrystals

Epicatechin:trigonelline is taken in mixture of ethanol: water solvents and heated up to 55° C.-58° C. until get a clear solution. Solution is kept at RT for 2-3 days to obtain desired co-crystal. The various parameters of the process to obtain co-crystals are listed below at Table 1. Different samples of epicatechin:trigonelline are analyzed as below:

TABLE 1
Various process parameters for preparation of the co-crystals.

| | | | Solvent | |
|---|---|---|---|---|
| | | Amount of | mixture for | |
| Compound | Co-crystal | the components | preparing the | Melting |
| number | molar ratio | in mg | cocrystal | point |
| 101 | (−)EPI- | 290 (EPI) 173 | 10 ml EtOH | 169-175° C. |
| | TRIG(1:1) | (trig) | 0.6 ml water | |
| 102 | (−)EPI- | 289 (EPI) | 10 ml EtOH | 170-176° C. |
| | TRIG(2:1) | 86 (trig) | 0.4 ml water | |
| 103 | (−)EPI- | 145 (EPI) 173 | 4 ml EtOH | 162-170° C. |
| | TRIG(1:2) | (trig) | 0.6 ml water | |
| 104 | (+)EPI- | 144 (EPI) | 4 ml EtOH | 165-178° C. |
| | TR1G(1:1) | 86 (trig) | 0.6 ml water | |
| 105 | (+)EPI- | 289 (EPI) | 10 ml EtOH | 158-178° C. |
| | TRIG(2:1) | 86 (trig) | 0.4 ml water | |
| 106 | (+)EPI- | 145 (EPI) 173 | 4 ml EtOH | 172-182° C. |
| | TRIG(1:2) | (trig) | 0.6 ml water | |

Melting Point trig: 258-260° C. 2, Melting Point (−)EPI: 239-240° C., Melting Point (+)EPI: 235-237° C.

### Example 2. Characteristics of the Co-Crystals of the Present Invention

### 2.1 Thermal Measurement (DSC)

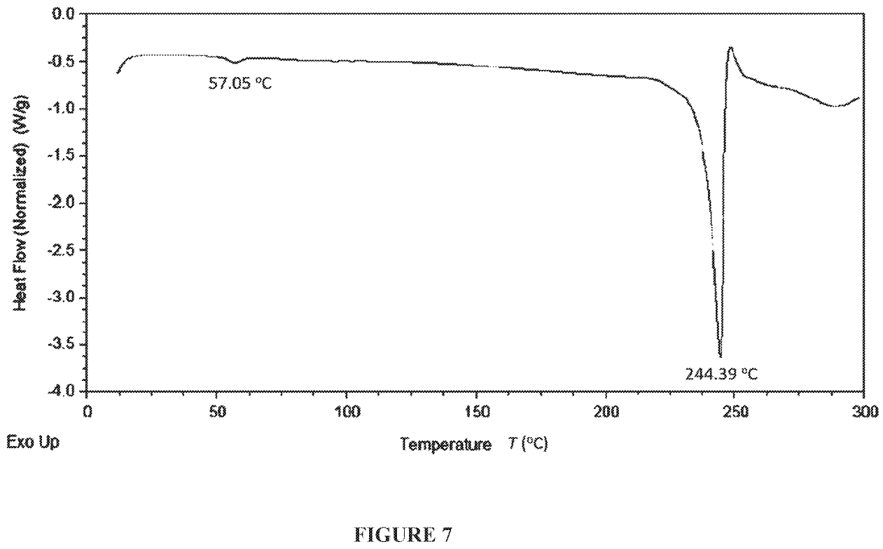

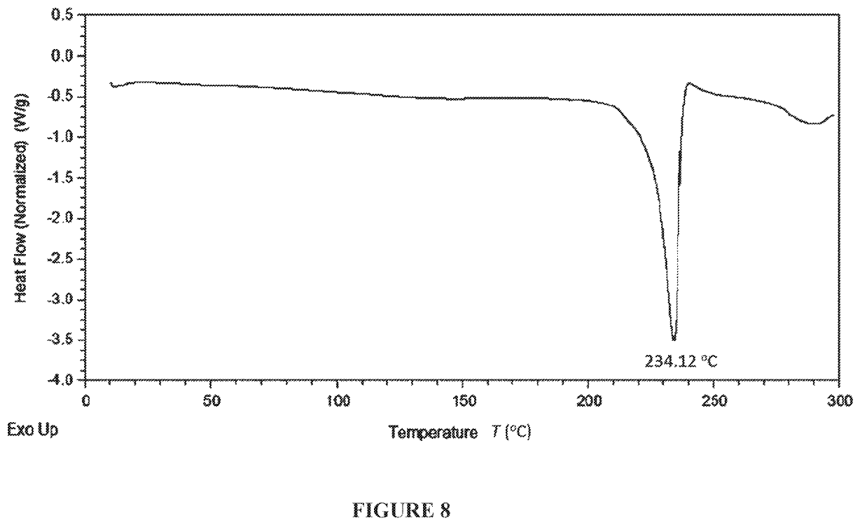

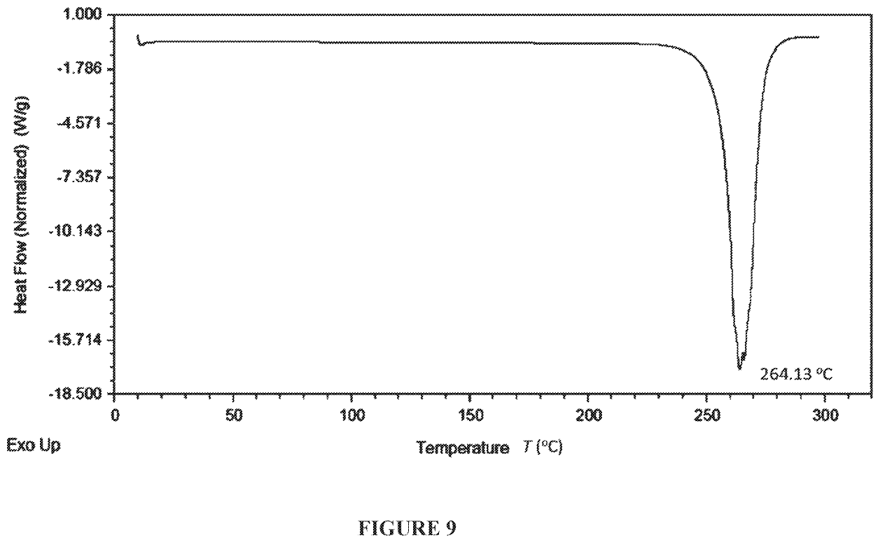

Thermal analysis of the samples was performed on a DSC apparatus (TA Instruments; Model: Discovery DSC 25 series) which was standardized for temperature and cell constants using indium. Samples (3-5 mg) crimped in the TZERO aluminum pan were analyzed from 10 to 300′C with a heating rate of 10° C./min. Samples were continuously removed with nitrogen at 50 ml/min. Unless otherwise indicated all reported transitions are as stated +/−10 degrees C. DSC pattern is depicted in FIGS. 1-9.

### 2.2 X Ray Diffraction Pattern

Co-crystals were analyzed using Rigaku Ultima IV Xray Diffractomcter (XRD). Scanning mode selected was 2 theta/theta and scan type was continuous. Parameters used during scanning is mentioned below:

X-Ray: 40 kV/20 mA

### DivSlit:⅔ deg

### DivH.L.Slit: 10 mm

### SctSlit: ⅔ deg

### RecSlit:0.3 mm

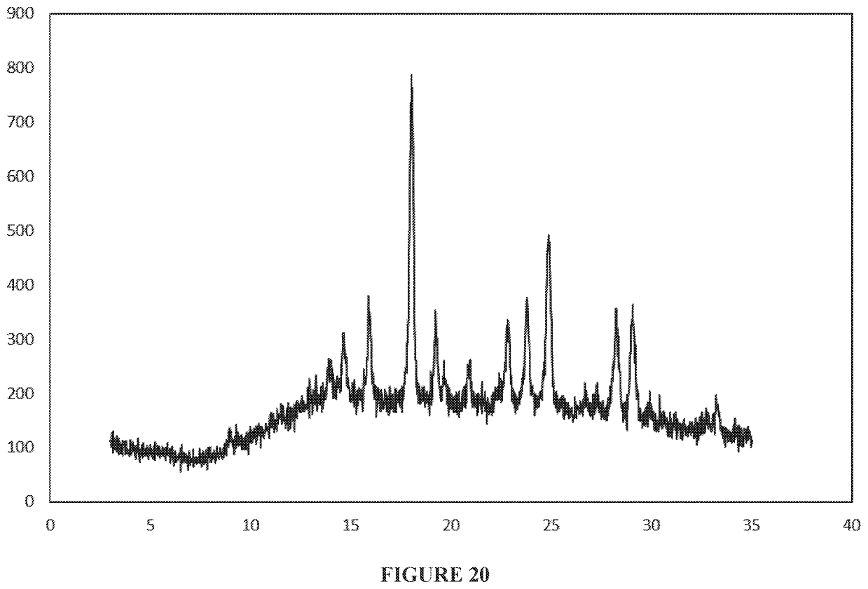

The results of X-ray diffraction are presented at FIGS. 10-12. The X-ray diffraction pattern of Compound 108 is shown in FIG. 20. The raw data of FIGS. 10-12 and 20 are shown in Table 2 below. 2 Theta values are listed side by side with their relative intensities.

TABLE 2
raw data of X-ray diffraction pattern
FIG. 10

| 2Theta | 101 | 2Theta | 101 | 2Theta | 101 | 2Theta | 101 | 2Theta | 101 |
|---|---|---|---|---|---|---|---|---|---|
| 3 | 138 | 3.84 | 114 | 4.68 | 120 | 5.52 | 145 | 6.36 | 155 |
| 3.02 | 131 | 3.86 | 121 | 4.7 | 135 | 5.54 | 166 | 6.38 | 164 |
| 3.04 | 148 | 3.88 | 120 | 4.72 | 128 | 5.56 | 164 | 6.4 | 148 |
| 3.06 | 116 | 3.9 | 122 | 4.74 | 115 | 5.58 | 146 | 6.42 | 153 |
| 3.08 | 132 | 3.92 | 129 | 4.76 | 120 | 5.6 | 124 | 6.44 | 174 |
| 3.1 | 139 | 3.94 | 107 | 4.78 | 121 | 5.62 | 158 | 6.46 | 183 |
| 3.12 | 150 | 3.96 | 125 | 4.8 | 122 | 5.64 | 151 | 6.48 | 170 |
| 3.14 | 114 | 3.98 | 110 | 4.82 | 117 | 5.66 | 137 | 6.5 | 160 |
| 3.16 | 124 | 4 | 115 | 4.84 | 136 | 5.68 | 143 | 6.52 | 168 |
| 3.18 | 132 | 4.02 | 125 | 4.86 | 145 | 5.7 | 154 | 6.54 | 168 |
| 3.2 | 110 | 4.04 | 108 | 4.88 | 120 | 5.72 | 146 | 6.56 | 173 |
| 3.22 | 115 | 4.06 | 112 | 4.9 | 148 | 5.74 | 156 | 6.58 | 161 |
| 3.24 | 104 | 4.08 | 114 | 4.92 | 111 | 5.76 | 142 | 6.6 | 161 |
| 3.26 | 114 | 4.1 | 110 | 4.94 | 142 | 5.78 | 151 | 6.62 | 160 |
| 3.28 | 114 | 4.12 | 122 | 4.96 | 142 | 5.8 | 164 | 6.64 | 173 |
| 3.3 | 129 | 4.14 | 115 | 4.98 | 114 | 5.82 | 188 | 6.66 | 163 |
| 3.32 | 114 | 4.16 | 125 | 5 | 126 | 5.84 | 175 | 6.68 | 169 |
| 3.34 | 120 | 4.18 | 136 | 5.02 | 140 | 5.86 | 159 | 6.7 | 177 |
| 3.36 | 138 | 4.2 | 127 | 5.04 | 143 | 5.88 | 122 | 6.72 | 162 |
| 3.38 | 127 | 4.22 | 128 | 5.06 | 122 | 5.9 | 149 | 6.74 | 182 |
| 3.4 | 120 | 4.24 | 110 | 5.08 | 124 | 5.92 | 156 | 6.76 | 157 |
| 3.42 | 130 | 4.26 | 136 | 5.1 | 145 | 5.94 | 152 | 6.78 | 176 |
| 3.44 | 128 | 4.28 | 128 | 5.12 | 135 | 5.96 | 149 | 6.8 | 205 |
| 3.46 | 131 | 4.3 | 141 | 5.14 | 148 | 5.98 | 139 | 6.82 | 193 |
| 3.48 | 119 | 4.32 | 119 | 5.16 | 138 | 6 | 169 | 6.84 | 185 |
| 3.5 | 123 | 4.34 | 126 | 5.18 | 153 | 6.02 | 161 | 6.86 | 214 |
| 3.52 | 119 | 4.36 | 111 | 5.2 | 121 | 6.04 | 160 | 6.88 | 228 |
| 3.54 | 118 | 4.38 | 129 | 5.22 | 144 | 6.06 | 150 | 6.9 | 250 |
| 3.56 | 121 | 4.4 | 126 | 5.24 | 156 | 6.08 | 157 | 6.92 | 208 |
| 3.58 | 132 | 4.42 | 127 | 5.26 | 131 | 6.1 | 142 | 6.94 | 246 |
| 3.6 | 116 | 4.44 | 102 | 5.28 | 128 | 6.12 | 146 | 6.96 | 202 |
| 3.62 | 110 | 4.46 | 113 | 5.3 | 152 | 6.14 | 129 | 6.98 | 181 |
| 3.64 | 113 | 4.48 | 120 | 5.32 | 147 | 6.16 | 138 | 7 | 163 |
| 3.66 | 122 | 4.5 | 114 | 5.34 | 122 | 6.18 | 170 | 7.02 | 162 |
| 3.68 | 120 | 4.52 | 120 | 5.36 | 157 | 6.2 | 160 | 7.04 | 154 |
| 3.7 | 123 | 4.54 | 139 | 5.38 | 138 | 6.22 | 154 | 7.06 | 163 |
| 3.72 | 127 | 4.56 | 120 | 5.4 | 167 | 6.24 | 180 | 7.08 | 159 |
| 3.74 | 119 | 4.58 | 136 | 5.42 | 133 | 6.26 | 151 | 7.1 | 191 |
| 3.76 | 107 | 4.6 | 118 | 5.44 | 142 | 6.28 | 173 | 7.12 | 159 |
| 3.78 | 124 | 4.62 | 117 | 5.46 | 139 | 6.3 | 162 | 7.14 | 152 |
| 3.8 | 129 | 4.64 | 133 | 5.48 | 140 | 6.32 | 156 | 7.16 | 158 |
| 3.82 | 112 | 4.66 | 128 | 5.5 | 141 | 6.34 | 148 | 7.18 | 166 |
| 7.2 | 170 | 8.04 | 176 | 8.88 | 173 | 9.72 | 206 | 10.56 | 208 |
| 7.22 | 182 | 8.06 | 190 | 8.9 | 176 | 9.74 | 210 | 10.58 | 238 |
| 7.24 | 182 | 8.08 | 198 | 8.92 | 189 | 9.76 | 226 | 10.6 | 255 |
| 7.26 | 163 | 8.1 | 171 | 8.94 | 186 | 9.78 | 227 | 10.62 | 256 |
| 7.28 | 175 | 8.12 | 181 | 8.96 | 188 | 9.8 | 192 | 10.64 | 209 |
| 7.3 | 159 | 8.14 | 185 | 8.98 | 195 | 9.82 | 203 | 10.66 | 229 |
| 7.32 | 170 | 8.16 | 175 | 9 | 197 | 9.84 | 202 | 10.68 | 223 |
| 7.34 | 148 | 8.18 | 175 | 9.02 | 199 | 9.86 | 234 | 10.7 | 211 |
| 7.36 | 172 | 8.2 | 184 | 9.04 | 196 | 9.88 | 210 | 10.72 | 230 |
| 7.38 | 160 | 8.22 | 145 | 9.06 | 179 | 9.9 | 209 | 10.74 | 221 |
| 7.4 | 198 | 8.24 | 183 | 9.08 | 200 | 9.92 | 243 | 10.76 | 218 |
| 7.42 | 186 | 8.26 | 195 | 9.1 | 207 | 9.94 | 196 | 10.78 | 199 |
| 7.44 | 149 | 8.28 | 186 | 9.12 | 219 | 9.96 | 203 | 10.8 | 205 |
| 7.46 | 187 | 8.3 | 192 | 9.14 | 204 | 9.98 | 224 | 10.82 | 224 |
| 7.48 | 175 | 8.32 | 190 | 9.16 | 194 | 10 | 226 | 10.84 | 204 |
| 7.5 | 183 | 8.34 | 202 | 9.18 | 196 | 10.02 | 224 | 10.86 | 236 |
| 7.52 | 190 | 8.36 | 179 | 9.2 | 181 | 10.04 | 175 | 10.88 | 272 |
| 7.54 | 165 | 8.38 | 181 | 9.22 | 181 | 10.06 | 189 | 10.9 | 240 |
| 7.56 | 169 | 8.4 | 164 | 9.24 | 201 | 10.08 | 221 | 10.92 | 219 |
| 7.58 | 170 | 8.42 | 187 | 9.26 | 199 | 10.1 | 201 | 10.94 | 219 |
| 7.6 | 167 | 8.44 | 186 | 9.28 | 218 | 10.12 | 216 | 10.96 | 267 |
| 7.62 | 155 | 8.46 | 167 | 9.3 | 220 | 10.14 | 226 | 10.98 | 238 |
| 7.64 | 158 | 8.48 | 167 | 9.32 | 195 | 10.16 | 203 | 11 | 231 |
| 7.66 | 160 | 8.5 | 204 | 9.34 | 226 | 10.18 | 200 | 11.02 | 233 |
| 7.68 | 177 | 8.52 | 184 | 9.36 | 206 | 10.2 | 219 | 11.04 | 252 |
| 7.7 | 153 | 8.54 | 178 | 9.38 | 225 | 10.22 | 228 | 11.06 | 219 |
| 7.72 | 185 | 8.56 | 153 | 9.4 | 216 | 10.24 | 210 | 11.08 | 259 |
| 7.74 | 164 | 8.58 | 178 | 9.42 | 202 | 10.26 | 211 | 11.1 | 268 |
| 7.76 | 210 | 8.6 | 193 | 9.44 | 213 | 10.28 | 248 | 11.12 | 260 |
| 7.78 | 200 | 8.62 | 178 | 9.46 | 208 | 10.3 | 208 | 11.14 | 270 |
| 7.8 | 152 | 8.64 | 183 | 9.48 | 200 | 10.32 | 219 | 11.16 | 324 |
| 7.82 | 182 | 8.66 | 195 | 9.5 | 216 | 10.34 | 218 | 11.18 | 306 |
| 7.84 | 214 | 8.68 | 160 | 9.52 | 205 | 10.36 | 231 | 11.2 | 386 |
| 7.86 | 147 | 8.7 | 171 | 9.54 | 193 | 10.38 | 220 | 11.22 | 318 |
| 7.88 | 173 | 8.72 | 203 | 9.56 | 215 | 10.4 | 222 | 11.24 | 331 |
| 7.9 | 179 | 8.74 | 167 | 9.58 | 191 | 10.42 | 202 | 11.26 | 302 |
| 7.92 | 175 | 8.76 | 181 | 9.6 | 197 | 10.44 | 236 | 11.28 | 273 |
| 7.94 | 166 | 8.78 | 206 | 9.62 | 247 | 10.46 | 213 | 11.3 | 241 |
| 7.96 | 152 | 8.8 | 200 | 9.64 | 204 | 10.48 | 227 | 11.32 | 265 |
| 7.98 | 187 | 8.82 | 166 | 9.66 | 241 | 10.5 | 235 | 11.34 | 249 |
| 8 | 161 | 8.84 | 189 | 9.68 | 191 | 10.52 | 208 | 11.36 | 207 |
| 8.02 | 170 | 8.86 | 173 | 9.7 | 207 | 10.54 | 210 | 11.38 | 218 |
| 11.4 | 239 | 12.24 | 260 | 13.08 | 279 | 13.92 | 246 | 14.76 | 277 |
| 11.42 | 254 | 12.2.6 | 228 | 13.1 | 286 | 13.94 | 282 | 14.78 | 288 |
| 11.44 | 227 | 12.28 | 264 | 13.12 | 293 | 13.96 | 259 | 14.8 | 310 |
| 11.46 | 225 | 12.3 | 258 | 13.14 | 267 | 13.98 | 299 | 14.82 | 284 |
| 11.48 | 255 | 12.32. | 252 | 13.16 | 279 | 14 | 284 | 14.84 | 315 |
| 11.5 | 243 | 12.34 | 235 | 13.18 | 253 | 14.02 | 244 | 14.86 | 309 |
| 11.52 | 224 | 12.36 | 264 | 13.2 | 294 | 14.04 | 302 | 14.88 | 329 |
| 11.54 | 226 | 12.38 | 262 | 13.22 | 309 | 14.06 | 267 | 14.9 | 326 |
| 11.56 | 230 | 12.4 | 272 | 13.24 | 273 | 14.08 | 303 | 14.92 | 349 |
| 11.58 | 231 | 12.42 | 235 | 13.26 | 295 | 14.1 | 305 | 14.94 | 310 |
| 11.6 | 226 | 12.44 | 273 | 13.28 | 289 | 14.12 | 245 | 14.96 | 290 |
| 11.62 | 208 | 12.46 | 250 | 13.3 | 303 | 14.14 | 271 | 14.98 | 352 |
| 11.64 | 247 | 12.48 | 248 | 13.32 | 328 | 14.16 | 301 | 15 | 289 |
| 11.66 | 237 | 12.5 | 258 | 13.34 | 316 | 14.18 | 296 | 15.02 | 253 |
| 11.68 | 247 | 12.52 | 254 | 13.36 | 312 | 14.2 | 289 | 15.04 | 285 |
| 11.7 | 247 | 12.54 | 245 | 13.38 | 309 | 14.22 | 247 | 15.06 | 286 |
| 11.72 | 223 | 12.56 | 265 | 13.4 | 373 | 14.24 | 311 | 15.08 | 265 |
| 11.74 | 246 | 12.58 | 277 | 13.42 | 393 | 14.26 | 265 | 15.1 | 283 |
| 11.76 | 238 | 12.6 | 284 | 13.44 | 369 | 14.28 | 286 | 15.12 | 281 |
| 11.78 | 268 | 12.62 | 266 | 13.46 | 347 | 14.3 | 246 | 15.14 | 246 |
| 11.8 | 241 | 12.64 | 269 | 13.48 | 387 | 14.32 | 298 | 15.16 | 261 |
| 11.82 | 249 | 12.66 | 286 | 13.5 | 409 | 14.34 | 295 | 15.18 | 252 |
| 11.84 | 258 | 12.68 | 245 | 13.52 | 364 | 14.36 | 272 | 15.2 | 280 |
| 11.86 | 249 | 12.7 | 245 | 13.54 | 437 | 14.38 | 284 | 15.22 | 275 |
| 11.88 | 210 | 12.72 | 267 | 13.56 | 429 | 14.4 | 291 | 15.24 | 261 |
| 11.9 | 260 | 12.74 | 263 | 13.58 | 529 | 14.42 | 308 | 15.26 | 219 |
| 11.92 | 296 | 12.76 | 283 | 13.6 | 606 | 14.44 | 338 | 15.28 | 237 |
| 11.94 | 250 | 12.78 | 274 | 13.62 | 656 | 14.46 | 347 | 15.3 | 278 |
| 11.96 | 283 | 12.8 | 253 | 13.64 | 708 | 14.48 | 343 | 15.32 | 265 |
| 11.98 | 239 | 12.82 | 268 | 13.66 | 582 | 14.5 | 349 | 15.34 | 271 |
| 12 | 249 | 12.84 | 255 | 13.68 | 464 | 14.52 | 358 | 15.36 | 273 |
| 12.02 | 255 | 12.86 | 269 | 13.7 | 369 | 14.54 | 366 | 15.38 | 246 |
| 12.04 | 269 | 12.88 | 286 | 13.72 | 330 | 14.56 | 316 | 15.4 | 282 |
| 12.06 | 251 | 12.9 | 252 | 13.74 | 275 | 14.58 | 295 | 15.42 | 293 |
| 12.08 | 255 | 12.92 | 279 | 13.76 | 294 | 14.6 | 273 | 15.44 | 223 |
| 12.1 | 233 | 12.94 | 265 | 13.78 | 289 | 14.62 | 291 | 15.46 | 298 |
| 12.12 | 250 | 12.96 | 258 | 13.8 | 311 | 14.64 | 300 | 15.48 | 276 |
| 12.14 | 231 | 12.98 | 268 | 13.82 | 299 | 14.66 | 291 | 15.5 | 291 |
| 12.16 | 233 | 13 | 277 | 13.84 | 314 | 14.68 | 305 | 15.52 | 269 |
| 12.18 | 240 | 13.02 | 264 | 13.86 | 287 | 14.7 | 292 | 15.54 | 287 |
| 12.2 | 272 | 13.04 | 275 | 13.88 | 292 | 14.72 | 305 | 15.56 | 277 |
| 12.22 | 286 | 13.06 | 263 | 13.9 | 319 | 14.74 | 266 | 15.58 | 289 |
| 15.6 | 307 | 16.44 | 466 | 17.28 | 234 | 18.12 | 315 | 18.96 | 715 |
| 15.62 | 286 | 16.46 | 439 | 17.3 | 274 | 18.14 | 269 | 18.98 | 764 |
| 15.64 | 273 | 16.48 | 443 | 17.32 | 275 | 18.16 | 272 | 19 | 680 |
| 15.66 | 257 | 16.5 | 312 | 17.34 | 309 | 18.18 | 266 | 19.02 | 550 |
| 15.68 | 282 | 16.52 | 290 | 17.36 | 319 | 18.2 | 316 | 19.04 | 499 |
| 15.7 | 256 | 16.54 | 275 | 17.38 | 292 | 18.22 | 290 | 19.06 | 360 |
| 15.72 | 273 | 16.56 | 263 | 17.4 | 316 | 18.24 | 235 | 19.08 | 278 |
| 15.74 | 251 | 16.58 | 320 | 17.42 | 309 | 18.26 | 266 | 19.1 | 313 |
| 15.76 | 315 | 16.6 | 287 | 17.44 | 319 | 18.28 | 251 | 19.12 | 271 |
| 15.78 | 278 | 16.62 | 276 | 17.46 | 298 | 18.3 | 293 | 19.14 | 280 |
| 15.8 | 281 | 16.64 | 255 | 17.48 | 320 | 18.32 | 247 | 19.16 | 280 |
| 15.82 | 271 | 16.66 | 275 | 17.5 | 345 | 18.34 | 279 | 19.18 | 284 |
| 15.84 | 273 | 16.68 | 259 | 17.52 | 378 | 18.36 | 258 | 19.2 | 254 |
| 15.86 | 295 | 16.7 | 265 | 17.54 | 367 | 18.38 | 264 | 19.22 | 280 |
| 15.88 | 272 | 16.72 | 275 | 17.56 | 412 | 18.4 | 257 | 19.24 | 283 |
| 15.9 | 304 | 16.74 | 263 | 17.58 | 495 | 18.42 | 285 | 19.26 | 290 |
| 15.92 | 264 | 16.76 | 248 | 17.6 | 574 | 18.44 | 263 | 19.28 | 298 |
| 15.94 | 296 | 16.78 | 267 | 17.62 | 797 | 18.46 | 242 | 19.3 | 268 |
| 15.96 | 264 | 16.8 | 275 | 17.64 | 972 | 18.48 | 265 | 19.32 | 302 |
| 15.98 | 282 | 16.82 | 249 | 17.66 | 903 | 18.5 | 251 | 19.34 | 296 |
| 16 | 256 | 16.84 | 254 | 17.68 | 734 | 18.52 | 288 | 19.36 | 270 |
| 16.02 | 273 | 16.86 | 249 | 17.7 | 579 | 18.54 | 253 | 19.38 | 278 |
| 16.04 | 278 | 16.88 | 265 | 17.72 | 487 | 18.56 | 266 | 19.4 | 266 |
| 16.06 | 298 | 16.9 | 252 | 17.74 | 411 | 18.58 | 246 | 19.42 | 230 |
| 16.08 | 293 | 16.92 | 269 | 17.76 | 321 | 18.6 | 261 | 19.44 | 265 |
| 16.1 | 285 | 16.94 | 242 | 17.78 | 347 | 18.62 | 252 | 19.46 | 247 |
| 16.12 | 300 | 16.96 | 265 | 17.8 | 354 | 18.64 | 240 | 19.48 | 247 |
| 16.14 | 280 | 16.98 | 253 | 17.82 | 373 | 18.66 | 289 | 19.5 | 225 |
| 16.16 | 269 | 17 | 272 | 17.84 | 350 | 18.68 | 267 | 19.52 | 252 |
| 16.18 | 294 | 17.02 | 251 | 17.86 | 422 | 18.7 | 270 | 19.54 | 279 |
| 16.2 | 271 | 17.04 | 269 | 17.88 | 424 | 18.72 | 266 | 19.56 | 267 |
| 16.22 | 301 | 17.06 | 284 | 17.9 | 456 | 18.74 | 276 | 19.58 | 278 |
| 16.24 | 290 | 17.08 | 254 | 17.92 | 475 | 18.76 | 329 | 19.6 | 288 |
| 16.26 | 334 | 17.1 | 299 | 17.94 | 591 | 18.78 | 325 | 19.62 | 244 |
| 16.28 | 274 | 17.12 | 284 | 17.96 | 682 | 18.8 | 335 | 19.64 | 250 |
| 16.3 | 270 | 17.14 | 301 | 17.98 | 822 | 18.82 | 335 | 19.66 | 285 |
| 16.32 | 316 | 17.16 | 252 | 18 | 843 | 18.84 | 328 | 19.68 | 281 |
| 16.34 | 307 | 17.18 | 287 | 18.02 | 734 | 18.86 | 329 | 19.7 | 234 |
| 16.36 | 350 | 17.2 | 291 | 18.04 | 592 | 18.88 | 417 | 19.72 | 287 |
| 16.38 | 399 | 17.22 | 272 | 18.06 | 482 | 18.9 | 433 | 19.74 | 230 |
| 16.4 | 390 | 17.24 | 262 | 18.08 | 357 | 18.92 | 485 | 19.76 | 246 |
| 16.42 | 434 | 17.26 | 285 | 18.1 | 301 | 18.94 | 585 | 19.78 | 269 |
| 19.8 | 291 | 20.64 | 290 | 21.48 | 308 | 22.32 | 341 | 23.16 | 310 |
| 19.82 | 287 | 20.66 | 293 | 21.5 | 269 | 22.34 | 335 | 23.18 | 265 |
| 19.84 | 270 | 20.68 | 272 | 21.52 | 305 | 22.36 | 319 | 23.2 | 312 |
| 19.86 | 258 | 20.7 | 310 | 21.54 | 273 | 22.38 | 303 | 23.22 | 279 |
| 19.88 | 258 | 20.72 | 284 | 21.56 | 272 | 22.4 | 328 | 23.24 | 276 |
| 19.9 | 294 | 20.74 | 286 | 21.58 | 269 | 22.42 | 319 | 23.26 | 324 |
| 19.92 | 283 | 20.76 | 320 | 21.6 | 258 | 22.44 | 328 | 23.28 | 267 |
| 19.94 | 245 | 20.78 | 340 | 21.62 | 258 | 22.46 | 360 | 23.3 | 265 |
| 19.96 | 251 | 20.8 | 358 | 21.64 | 280 | 22.48 | 413 | 23.32 | 284 |
| 19.98 | 289 | 20.82 | 340 | 21.66 | 279 | 22.5 | 465 | 23.34 | 291 |
| 20 | 247 | 20.84 | 402 | 21.68 | 279 | 22.52 | 454 | 23.36 | 295 |
| 20.02 | 277 | 20.86 | 438 | 21.7 | 253 | 22.54 | 439 | 23.38 | 283 |
| 20.04 | 276 | 20.88 | 388 | 21.72 | 287 | 22.56 | 411 | 23.4 | 272 |
| 20.06 | 264 | 20.9 | 382 | 21.74 | 283 | 22.58 | 380 | 23.42 | 289 |
| 20.08 | 283 | 20.92 | 360 | 21.76 | 266 | 22.6 | 328 | 23.44 | 291 |
| 20.1 | 285 | 20.94 | 326 | 21.78 | 283 | 22.62 | 315 | 23.46 | 292 |
| 20.12 | 288 | 20.96 | 305 | 21.8 | 285 | 22.64 | 313 | 23.48 | 278 |
| 20.14 | 262 | 20.98 | 310 | 21.82 | 273 | 22.66 | 336 | 23.5 | 327 |
| 20.16 | 297 | 21 | 305 | 21.84 | 272 | 22.68 | 352 | 23.52 | 369 |
| 20.18 | 289 | 21.02 | 302 | 21.86 | 279 | 22.7 | 329 | 23.54 | 399 |
| 20.2 | 289 | 21.04 | 290 | 21.88 | 269 | 22.72 | 323 | 23.56 | 396 |
| 20.22 | 278 | 21.06 | 281 | 21.9 | 271 | 22.74 | 307 | 23.58 | 450 |
| 20.24 | 268 | 21.08 | 280 | 21.92 | 289 | 22.76 | 270 | 23.6 | 440 |
| 20.26 | 256 | 21.1 | 299 | 21.94 | 258 | 22.78 | 289 | 23.62 | 417 |
| 20.28 | 277 | 21.12 | 320 | 21.96 | 267 | 22.8 | 244 | 23.64 | 357 |
| 20.3 | 295 | 21.14 | 317 | 21.98 | 298 | 22.82 | 279 | 23.66 | 337 |
| 20.32 | 299 | 21.16 | 331 | 22 | 278 | 22.84 | 295 | 23.68 | 314 |
| 20.34 | 258 | 21.18 | 323 | 22.02 | 275 | 22.86 | 266 | 23.7 | 304 |
| 20.36 | 282 | 21.2 | 284 | 22.04 | 268 | 22.88 | 268 | 23.72 | 283 |
| 20.38 | 279 | 21.22 | 288 | 22.06 | 272 | 22.9 | 269 | 23.74 | 284 |
| 20.4 | 271 | 21.24 | 282 | 22.08 | 308 | 22.92 | 262 | 23.76 | 326 |
| 20.42 | 269 | 21.26 | 277 | 22.1 | 300 | 22.94 | 308 | 23.78 | 261 |
| 20.44 | 259 | 21.28 | 290 | 22.12 | 284 | 22.96 | 266 | 23.8 | 283 |
| 20.46 | 277 | 21.3 | 332 | 22.14 | 293 | 22.98 | 278 | 23.82 | 291 |
| 20.48 | 263 | 21.32 | 273 | 22.16 | 293 | 23 | 257 | 23.84 | 291 |
| 20.5 | 304 | 21.34 | 338 | 22.18 | 309 | 23.02 | 281 | 23.86 | 301 |
| 20.52 | 293 | 21.36 | 307 | 22.2 | 320 | 23.04 | 270 | 23.88 | 282 |
| 20.54 | 278 | 21.38 | 384 | 22.22 | 358 | 23.06 | 267 | 23.9 | 264 |
| 20.56 | 266 | 21.4 | 372 | 22.24 | 377 | 23.08 | 282 | 23.92 | 289 |
| 20.58 | 280 | 21.42 | 398 | 22.26 | 345 | 23.1 | 271 | 23.94 | 252 |
| 20.6 | 303 | 21.44 | 373 | 22.28 | 365 | 23.12 | 296 | 23.96 | 289 |
| 20.62 | 269 | 21.46 | 315 | 22.3 | 310 | 23.14 | 302 | 23.98 | 275 |
| 24 | 256 | 24.84 | 305 | 25.68 | 297 | 26.52 | 245 | 27.36 | 250 |
| 24.02 | 284 | 24.86 | 289 | 25.7 | 352 | 26.54 | 237 | 27.38 | 283 |
| 24.04 | 289 | 24.88 | 286 | 25.72 | 377 | 26.56 | 258 | 27.4 | 246 |
| 24.06 | 256 | 24.9 | 297 | 25.74 | 438 | 26.58 | 259 | 27.42 | 240 |
| 24.08 | 261 | 24.92 | 279 | 25.76 | 446 | 26.6 | 266 | 27.44 | 304 |
| 24.1 | 302 | 24.94 | 307 | 25.78 | 415 | 26.62 | 276 | 27.46 | 303 |
| 24.12 | 322 | 24.96 | 283 | 25.8 | 426 | 26.64 | 261 | 27.48 | 284 |
| 24.14 | 310 | 24.98 | 364 | 25.82 | 406 | 26.66 | 286 | 27.5 | 315 |
| 24.16 | 288 | 25 | 372 | 25.84 | 345 | 26.68 | 253 | 27.52 | 336 |
| 24.18 | 310 | 25.02 | 391 | 25.86 | 308 | 26.7 | 226 | 27.54 | 325 |
| 24.2 | 273 | 25.04 | 420 | 25.88 | 310 | 26.72 | 266 | 27.56 | 299 |
| 24.22 | 291 | 25.06 | 374 | 25.9 | 302 | 26.74 | 269 | 27.58 | 287 |
| 24.24 | 323 | 25.08 | 393 | 25.92 | 270 | 26.76 | 291 | 27.6 | 245 |
| 24.26 | 283 | 25.1 | 342 | 25.94 | 272 | 26.78 | 274 | 27.62 | 262 |
| 24.28 | 278 | 25.12 | 308 | 25.96 | 258 | 26.8 | 283 | 27.64 | 263 |
| 24.3 | 250 | 25.14 | 302 | 25.98 | 302 | 26.82 | 272 | 27.66 | 261 |
| 24.32 | 302 | 25.16 | 298 | 26 | 283 | 26.84 | 332 | 27.68 | 276 |
| 24.34 | 284 | 25.18 | 236 | 26.02 | 272 | 26.86 | 338 | 27.7 | 247 |
| 24.36 | 267 | 25.2 | 301 | 26.04 | 266 | 26.88 | 396 | 27.72 | 249 |
| 24.38 | 264 | 25.22 | 306 | 26.06 | 253 | 26.9 | 439 | 27.74 | 219 |
| 24.4 | 287 | 25.24 | 304 | 26.08 | 270 | 26.92 | 472 | 27.76 | 207 |
| 24.42 | 266 | 25.26 | 317 | 26.1 | 236 | 26.94 | 466 | 27.78 | 260 |
| 24.44 | 294 | 25.28 | 254 | 26.12 | 237 | 26.96 | 452 | 27.8 | 265 |
| 24.46 | 258 | 25.3 | 315 | 26.14 | 253 | 26.98 | 450 | 27.82 | 241 |
| 24.48 | 266 | 25.32 | 339 | 26.16 | 245 | 27 | 496 | 27.84 | 247 |
| 24.5 | 298 | 25.34 | 316 | 26.18 | 267 | 27.02 | 448 | 27.86 | 308 |
| 24.52 | 245 | 25.36 | 328 | 26.2 | 263 | 27.04 | 438 | 27.88 | 315 |
| 24.54 | 253 | 25.38 | 317 | 26.22 | 258 | 27.06 | 465 | 27.9 | 344 |
| 24.56 | 274 | 25.4 | 300 | 26.24 | 227 | 27.08 | 406 | 27.92 | 366 |
| 24.58 | 279 | 25.42 | 313 | 26.26 | 263 | 27.1 | 342 | 27.94 | 325 |
| 24.6 | 269 | 25.44 | 272 | 26.28 | 220 | 27.12 | 330 | 27.96 | 301 |
| 24.62 | 271 | 25.46 | 282 | 26.3 | 259 | 27.14 | 304 | 27.98 | 290 |
| 24.64 | 266 | 25.48 | 301 | 26.32 | 267 | 27.16 | 305 | 28 | 306 |
| 24.66 | 304 | 25.5 | 268 | 26.34 | 261 | 27.18 | 270 | 28.02 | 257 |
| 24.68 | 259 | 25.52 | 313 | 26.36 | 249 | 27.2 | 276 | 28.04 | 259 |
| 24.7 | 280 | 25.54 | 291 | 26.38 | 254 | 27.22 | 285 | 28.06 | 234 |
| 24.72 | 271 | 25.56 | 276 | 26.4 | 250 | 27.24 | 230 | 28.08 | 267 |
| 24.74 | 307 | 25.58 | 296 | 26.42 | 247 | 27.26 | 275 | 28.1 | 248 |
| 24.76 | 283 | 25.6 | 278 | 26.44 | 247 | 27.28 | 252 | 28.12 | 209 |
| 24.78 | 304 | 25.62 | 292 | 26.46 | 249 | 27.3 | 261 | 28.14 | 259 |
| 24.8 | 281 | 25.64 | 278 | 26.48 | 243 | 27.32 | 250 | 28.16 | 250 |
| 24.82 | 270 | 25.66 | 287 | 26.5 | 249 | 27.34 | 260 | 28.18 | 241 |
| 28.2 | 234 | 29.04 | 396 | 29.88 | 207 | 30.72 | 217 | 31.56 | 215 |
| 28.22 | 230 | 29.06 | 417 | 29.9 | 233 | 30.74 | 197 | 31.58 | 203 |
| 28.24 | 243 | 29.08 | 373 | 29.92 | 234 | 30.76 | 198 | 31.6 | 205 |
| 28.26 | 237 | 29.1 | 353 | 29.94 | 243 | 30.78 | 239 | 31.62 | 215 |
| 28.28 | 243 | 29.12 | 343 | 29.96 | 230 | 30.8 | 223 | 31.64 | 190 |
| 28.3 | 251 | 29.14 | 342 | 29.98 | 225 | 30.82 | 202 | 31.66 | 193 |
| 28.32 | 232 | 29.16 | 350 | 30 | 210 | 30.84 | 223 | 31.68 | 224 |
| 28.34 | 257 | 29.18 | 286 | 30.02 | 221 | 30.86 | 223 | 31.7 | 175 |
| 28.36 | 255 | 29.2 | 242 | 30.04 | 221 | 30.88 | 218 | 31.72 | 202 |
| 28.38 | 248 | 29.22 | 273 | 30.06 | 247 | 30.9 | 189 | 31.74 | 220 |
| 28.4 | 285 | 29.24 | 260 | 30.08 | 227 | 30.92 | 209 | 31.76 | 199 |
| 28.42 | 287 | 29.26 | 280 | 30.1 | 227 | 30.94 | 205 | 31.78 | 228 |
| 28.44 | 277 | 29.28 | 354 | 30.12 | 235 | 30.96 | 195 | 31.8 | 202 |
| 28.46 | 297 | 29.3 | 322 | 30.14 | 211 | 30.98 | 218 | 31.82 | 195 |
| 28.48 | 340 | 29.32 | 312 | 30.16 | 237 | 31 | 223 | 31.84 | 162 |
| 28.5 | 336 | 29.34 | 335 | 30.18 | 222 | 31.02 | 191 | 31.86 | 205 |
| 28.52 | 350 | 29.36 | 334 | 30.2 | 254 | 31.04 | 215 | 31.88 | 207 |
| 28.54 | 332 | 29.38 | 300 | 30.22 | 221 | 31.06 | 204 | 31.9 | 192 |
| 28.56 | 293 | 29.4 | 282 | 30.24 | 234 | 31.08 | 187 | 31.92 | 176 |
| 28.58 | 271 | 29.42 | 261 | 30.26 | 295 | 31.1 | 224 | 31.94 | 211 |
| 28.6 | 301 | 29.44 | 258 | 30.28 | 263 | 31.12 | 202 | 31.96 | 189 |
| 28.62 | 310 | 29.46 | 247 | 30.3 | 250 | 31.14 | 209 | 31.98 | 200 |
| 28.64 | 274 | 29.48 | 256 | 30.32 | 245 | 31.16 | 187 | 32 | 184 |
| 28.66 | 284 | 29.5 | 222 | 30.34 | 274 | 31.18 | 205 | 32.02 | 208 |
| 28.68 | 259 | 29.52 | 238 | 30.36 | 253 | 31.2 | 218 | 32.04 | 178 |
| 28.7 | 273 | 29.54 | 232 | 30.38 | 250 | 31.22 | 215 | 32.06 | 172 |
| 28.72 | 266 | 29.56 | 242 | 30.4 | 249 | 31.24 | 205 | 32.08 | 189 |
| 28.74 | 233 | 29.58 | 210 | 30.42 | 247 | 31.26 | 220 | 32.1 | 182 |
| 28.76 | 236 | 29.6 | 225 | 30.44 | 231 | 31.28 | 233 | 32.12 | 182 |
| 28.78 | 218 | 29.62 | 225 | 30.46 | 245 | 31.3 | 233 | 32.14 | 186 |
| 28.8 | 221 | 29.64 | 248 | 30.48 | 235 | 31.32 | 247 | 32.16 | 173 |
| 28.82 | 226 | 29.66 | 225 | 30.5 | 188 | 31.34 | 286 | 32.18 | 189 |
| 28.84 | 267 | 29.68 | 242 | 30.52 | 212 | 31.36 | 229 | 32.2 | 197 |
| 28.86 | 251 | 29.7 | 240 | 30.54 | 218 | 31.38 | 237 | 32.22 | 194 |
| 28.88 | 260 | 29.72 | 223 | 30.56 | 244 | 31.4 | 231 | 32.24 | 180 |
| 28.9 | 288 | 29.74 | 227 | 30.58 | 209 | 31.42 | 225 | 32.26 | 196 |
| 28.92 | 251 | 29.76 | 218 | 30.6 | 199 | 31.44 | 206 | 32.28 | 217 |
| 28.94 | 243 | 29.78 | 237 | 30.62 | 215 | 31.46 | 199 | 32.3 | 207 |
| 28.96 | 300 | 29.8 | 196 | 30.64 | 213 | 31.48 | 206 | 32.32 | 199 |
| 28.98 | 298 | 29.82 | 235 | 30.66 | 194 | 31.5 | 214 | 32.34 | 213 |
| 29 | 324 | 29.84 | 226 | 30.68 | 201 | 31.52 | 231 | 32.36 | 229 |
| 29.02 | 340 | 29.86 | 233 | 30.7 | 236 | 31.54 | 219 | 32.38 | 188 |
| 32.4 | 210 | 33.24 | 210 | 34.08 | 203 | 34.92 | 165 | | |
| 32.42 | 214 | 33.26 | 191 | 34.1 | 196 | 34.94 | 159 | | |
| 32.44 | 202 | 33.28 | 183 | 34.12 | 223 | 34.96 | 166 | | |
| 32.46 | 218 | 33.3 | 177 | 34.14 | 220 | 34.98 | 150 | | |
| 32.48 | 230 | 33.32 | 177 | 34.16 | 196 | 35 | 204 | | |
| 32.5 | 196 | 33.34 | 182 | 34.18 | 218 | | | | |
| 32.52 | 228 | 33.36 | 175 | 34.2 | 200 | | | | |
| 32.54 | 226 | 33.38 | 191 | 34.22 | 213 | | | | |
| 32.56 | 184 | 33.4 | 170 | 34.24 | 168 | | | | |
| 32.58 | 196 | 33.42 | 196 | 34.26 | 169 | | | | |
| 32.6 | 208 | 33.44 | 195 | 34.28 | 164 | | | | |
| 32.62 | 198 | 33.46 | 210 | 34.3 | 149 | | | | |
| 32.64 | 208 | 33.48 | 152 | 34.32 | 184 | | | | |
| 32.66 | 186 | 33.5 | 156 | 34.34 | 160 | | | | |
| 32.68 | 194 | 33.52 | 185 | 34.36 | 155 | | | | |
| 32.7 | 174 | 33.54 | 171 | 34.38 | 172 | | | | |
| 32.72 | 197 | 33.56 | 197 | 34.4 | 165 | | | | |
| 32.74 | 174 | 33.58 | 175 | 34.42 | 183 | | | | |
| 32.76 | 184 | 33.6 | 161 | 34.44 | 153 | | | | |
| 32.78 | 176 | 33.62 | 151 | 34.46 | 172 | | | | |
| 32.8 | 175 | 33.64 | 211 | 34.48 | 173 | | | | |
| 32.82 | 194 | 33.66 | 181 | 34.5 | 152 | | | | |
| 32.84 | 167 | 33.68 | 168 | 34.52 | 174 | | | | |
| 32.86 | 206 | 33.7 | 183 | 34.54 | 189 | | | | |
| 32.88 | 179 | 33.72 | 159 | 34.56 | 160 | | | | |
| 32.9 | 179 | 33.74 | 179 | 34.58 | 172 | | | | |
| 32.92 | 170 | 33.76 | 168 | 34.6 | 161 | | | | |
| 32.94 | 178 | 33.78 | 162 | 34.62 | 180 | | | | |
| 32.96 | 177 | 33.8 | 210 | 34.64 | 166 | | | | |
| 32.98 | 195 | 33.82 | 164 | 34.66 | 171 | | | | |
| 33 | 196 | 33.84 | 208 | 34.68 | 172 | | | | |
| 33.02 | 175 | 33.86 | 194 | 34.7 | 182 | | | | |
| 33.04 | 181 | 33.88 | 172 | 34.72 | 161 | | | | |
| 33.06 | 171 | 33.9 | 204 | 34.74 | 169 | | | | |
| 33.08 | 185 | 33.92 | 168 | 34.76 | 185 | | | | |
| 33.1 | 204 | 33.94 | 198 | 34.78 | 159 | | | | |
| 33.12 | 191 | 33.96 | 189 | 34.8 | 182 | | | | |
| 33.14 | 184 | 33.98 | 226 | 34.82 | 157 | | | | |
| 33.16 | 179 | 34 | 241 | 34.84 | 171 | | | | |
| 33.18 | 207 | 34.02 | 237 | 34.86 | 167 | | | | |
| 33.2 | 191 | 34.04 | 234 | 34.88 | 176 | | | | |
| 33.22 | 201 | 34.06 | 228 | 34.9 | 171 | | | | |

FIG. 11

| 2Theta | 104 | 2Theta | 104 | 2Theta | 104 | 2Theta | 104 | 2Theta | 104 |
|---|---|---|---|---|---|---|---|---|---|
| 3 | 129 | 3.84 | 104 | 4.68 | 107 | 5.52 | 120 | 6.36 | 136 |
| 3.02 | 109 | 3.86 | 94 | 4.7 | 105 | 5.54 | 93 | 6.38 | 132 |
| 3.04 | 108 | 3.88 | 92 | 4.72 | 100 | 5.56 | 114 | 6.4 | 133 |
| 3.06 | 113 | 3.9 | 83 | 4.74 | 108 | 5.58 | 126 | 6.42 | 149 |
| 3.08 | 120 | 3.92 | 93 | 4.76 | 99 | 5.6 | 151 | 6.44 | 146 |
| 3.1 | 131 | 3.94 | 103 | 4.78 | 114 | 5.62 | 118 | 6.46 | 121 |
| 3.12 | 92 | 3.96 | 91 | 4.8 | 121 | 5.64 | 97 | 6.48 | 147 |
| 3.14 | 96 | 3.98 | 104 | 4.82 | 111 | 5.66 | 128 | 6.5 | 143 |
| 3.16 | 106 | 4 | 117 | 4.84 | 96 | 5.68 | 121 | 6.52 | 142 |
| 3.18 | 132 | 4.02 | 100 | 4.86 | 100 | 5.7 | 114 | 6.54 | 140 |
| 3.2 | 122 | 4.04 | 117 | 4.88 | 110 | 5.72 | 129 | 6.56 | 149 |
| 3.22 | 90 | 4.06 | 96 | 4.9 | 93 | 5.74 | 120 | 6.58 | 127 |
| 3.24 | 112 | 4.08 | 96 | 4.92 | 116 | 5.76 | 119 | 6.6 | 135 |
| 3.26 | 90 | 4.1 | 118 | 4.94 | 113 | 5.78 | 122 | 6.62 | 147 |
| 3.28 | 113 | 4.12 | 113 | 4.96 | 104 | 5.8 | 106 | 6.64 | 140 |
| 3.3 | 100 | 4.14 | 113 | 4.98 | 122 | 5.82 | 121 | 6.66 | 132 |
| 3.32 | 87 | 4.16 | 101 | 5 | 95 | 5.84 | 114 | 6.68 | 152 |
| 3.34 | 99 | 4.18 | 114 | 5.02 | 127 | 5.86 | 113 | 6.7 | 145 |
| 3.36 | 117 | 4.2 | 93 | 5.04 | 104 | 5.88 | 119 | 6.72 | 162 |
| 3.38 | 100 | 4.22 | 118 | 5.06 | 127 | 5.9 | 141 | 6.74 | 174 |
| 3.4 | 117 | 4.24 | 94 | 5.08 | 123 | 5.92 | 133 | 6.76 | 144 |
| 3.42 | 114 | 4.26 | 103 | 5.1 | 112 | 5.94 | 110 | 6.78 | 155 |
| 3.44 | 99 | 4.28 | 112 | 5.12 | 119 | 5.96 | 144 | 6.8 | 145 |
| 3.46 | 96 | 4.3 | 114 | 5.14 | 126 | 5.98 | 140 | 6.82 | 152 |
| 3.48 | 100 | 4.32 | 96 | 5.16 | 118 | 6 | 122 | 6.84 | 151 |
| 3.5 | 84 | 4.34 | 104 | 5.18 | 123 | 6.02 | 144 | 6.86 | 142 |
| 3.52 | 89 | 4.36 | 104 | 5.2 | 109 | 6.04 | 132 | 6.88 | 164 |
| 3.54 | 115 | 4.38 | 111 | 5.22 | 114 | 6.06 | 135 | 6.9 | 224 |
| 3.56 | 97 | 4.4 | 78 | 5.24 | 108 | 6.08 | 141 | 6.92 | 334 |
| 3.58 | 90 | 4.42 | 97 | 5.26 | 118 | 6.1 | 148 | 6.94 | 333 |
| 3.6 | 107 | 4.44 | 101 | 5.28 | 102 | 6.12 | 134 | 6.96 | 245 |
| 3.62 | 96 | 4.46 | 116 | 5.3 | 107 | 6.14 | 132 | 6.98 | 151 |
| 3.64 | 91 | 4.48 | 99 | 5.32 | 82 | 6.16 | 134 | 7 | 151 |
| 3.66 | 101 | 4.5 | 115 | 5.34 | 116 | 6.18 | 132 | 7.02 | 165 |
| 3.68 | 126 | 4.52 | 93 | 5.36 | 130 | 6.2 | 141 | 7.04 | 146 |
| 3.7 | 107 | 4.54 | 105 | 5.38 | 117 | 6.22 | 158 | 7.06 | 162 |
| 3.72 | 111 | 4.56 | 110 | 5.4 | 111 | 6.24 | 127 | 7.08 | 126 |
| 3.74 | 89 | 4.58 | 101 | 5.42 | 119 | 6.26 | 147 | 7.1 | 156 |
| 3.76 | 103 | 4.6 | 104 | 5.44 | 94 | 6.28 | 157 | 7.12 | 149 |
| 3.78 | 94 | 4.62 | 122 | 5.46 | 131 | 6.3 | 116 | 7.14 | 131 |
| 3.8 | 109 | 4.64 | 107 | 5.48 | 129 | 6.32 | 136 | 7.16 | 168 |
| 3.82 | 99 | 4.66 | 93 | 5.5 | 109 | 6.34 | 134 | 7.18 | 133 |
| 7.2 | 154 | 8.04 | 140 | 8.88 | 176 | 9.72 | 199 | 10.56 | 230 |
| 7.22 | 131 | 8.06 | 169 | 8.9 | 188 | 9.74 | 175 | 10.58 | 216 |
| 7.24 | 139 | 8.08 | 165 | 8.92 | 165 | 9.76 | 207 | 10.6 | 226 |
| 7.26 | 139 | 8.1 | 159 | 8.94 | 175 | 9.78 | 197 | 10.62 | 209 |
| 7.28 | 152 | 8.12 | 148 | 8.96 | 183 | 9.8 | 215 | 10.64 | 188 |
| 7.3 | 141 | 8.14 | 135 | 8.98 | 180 | 9.82 | 184 | 10.66 | 206 |
| 7.32 | 146 | 8.16 | 143 | 9 | 167 | 9.84 | 211 | 10.68 | 215 |
| 7.34 | 162 | 8.18 | 165 | 9.02 | 183 | 9.86 | 217 | 10.7 | 200 |
| 7.36 | 149 | 8.2 | 159 | 9.04 | 187 | 9.88 | 208 | 10.72 | 214 |
| 7.38 | 143 | 8.22 | 163 | 9.06 | 189 | 9.9 | 182 | 10.74 | 192 |
| 7.4 | 148 | 8.24 | 160 | 9.08 | 155 | 9.92 | 190 | 10.76 | 205 |
| 7.42 | 138 | 8.26 | 164 | 9.1 | 192 | 9.94 | 211 | 10.78 | 231 |
| 7.44 | 165 | 8.28 | 172 | 9.12 | 185 | 9.96 | 208 | 10.8 | 197 |
| 7.46 | 155 | 8.3 | 179 | 9.14 | 189 | 9.98 | 204 | 10.82 | 182 |
| 7.48 | 138 | 8.32 | 171 | 9.16 | 178 | 10 | 204 | 10.84 | 223 |
| 7.5 | 150 | 8.34 | 141 | 9.18 | 177 | 10.02 | 194 | 10.86 | 210 |
| 7.52 | 143 | 8.36 | 176 | 9.2 | 181 | 10.04 | 196 | 10.88 | 189 |
| 7.54 | 135 | 8.38 | 174 | 9.22 | 181 | 10.06 | 205 | 10.9 | 193 |
| 7.56 | 135 | 8.4 | 162 | 9.24 | 197 | 10.08 | 162 | 10.92 | 184 |
| 7.58 | 159 | 8.42 | 173 | 9.26 | 198 | 10.1 | 205 | 10.94 | 218 |
| 7.6 | 167 | 8.44 | 179 | 9.28 | 191 | 10.12 | 215 | 10.96 | 212 |
| 7.62 | 137 | 8.46 | 167 | 9.3 | 169 | 10.14 | 201 | 10.98 | 218 |
| 7.64 | 167 | 8.48 | 172 | 9.32 | 177 | 10.16 | 196 | 11 | 209 |
| 7.66 | 145 | 8.5 | 153 | 9.34 | 177 | 10.18 | 215 | 11.02 | 238 |
| 7.68 | 143 | 8.52 | 181 | 9.36 | 210 | 10.2 | 218 | 11.04 | 232 |
| 7.7 | 155 | 8.54 | 172 | 9.38 | 191 | 10.22 | 197 | 11.06 | 222 |
| 7.72 | 132 | 8.56 | 170 | 9.4 | 181 | 10.24 | 189 | 11.08 | 234 |
| 7.74 | 140 | 8.58 | 192 | 9.42 | 192 | 10.26 | 214 | 11.1 | 223 |
| 7.76 | 147 | 8.6 | 147 | 9.44 | 191 | 10.28 | 210 | 11.12 | 223 |
| 7.78 | 163 | 8.62 | 161 | 9.46 | 188 | 10.3 | 189 | 11.14 | 222 |
| 7.8 | 163 | 8.64 | 163 | 9.48 | 182 | 10.32 | 211 | 11.16 | 255 |
| 7.82 | 143 | 8.66 | 161 | 9.5 | 209 | 10.34 | 210 | 11.18 | 276 |
| 7.84 | 163 | 8.68 | 147 | 9.52 | 205 | 10.36 | 212 | 11.2 | 300 |
| 7.86 | 160 | 8.7 | 186 | 9.54 | 209 | 10.38 | 207 | 11.22 | 301 |
| 7.88 | 150 | 8.72 | 185 | 9.56 | 193 | 10.4 | 204 | 11.24 | 257 |
| 7.9 | 159 | 8.74 | 175 | 9.58 | 188 | 10.42 | 208 | 11.26 | 237 |
| 7.92 | 146 | 8.76 | 161 | 9.6 | 184 | 10.44 | 207 | 11.28 | 231 |
| 7.94 | 153 | 8.78 | 180 | 9.62 | 188 | 10.46 | 221 | 11.3 | 212 |
| 7.96 | 159 | 8.8 | 182 | 9.64 | 190 | 10.48 | 228 | 11.32 | 208 |
| 7.98 | 151 | 8.82 | 156 | 9.66 | 180 | 10.5 | 195 | 11.34 | 220 |
| 8 | 166 | 8.84 | 183 | 9.68 | 201 | 10.52 | 207 | 11.36 | 245 |
| 8.02 | 140 | 8.86 | 159 | 9.7 | 174 | 10.54 | 196 | 11.38 | 202 |
| 11.4 | 200 | 12.24 | 240 | 13.08 | 260 | 13.92 | 280 | 14.76 | 269 |
| 11.42 | 217 | 12.26 | 228 | 13.1 | 245 | 13.94 | 259 | 14.78 | 270 |
| 11.44 | 193 | 12.28 | 214 | 13.12 | 266 | 13.96 | 252 | 14.8 | 270 |
| 11.46 | 232 | 12.3 | 224 | 13.14 | 271 | 13.98 | 250 | 14.82 | 249 |
| 11.48 | 231 | 12.32 | 253 | 13.16 | 257 | 14 | 265 | 14.84 | 257 |
| 11.5 | 222 | 12.34 | 236 | 13.18 | 262 | 14.02 | 255 | 14.86 | 250 |
| 11.52 | 235 | 12.36 | 240 | 13.2 | 276 | 14.04 | 269 | 14.88 | 291 |
| 11.54 | 227 | 12.38 | 237 | 13.22 | 254 | 14.06 | 266 | 14.9 | 258 |
| 11.56 | 233 | 12.4 | 219 | 13.24 | 289 | 14.08 | 253 | 14.92 | 273 |
| 11.58 | 246 | 12.42 | 252 | 13.26 | 228 | 14.1 | 242 | 14.94 | 241 |
| 11.6 | 229 | 12.44 | 262 | 13.28 | 277 | 14.12 | 267 | 14.96 | 229 |
| 11.62 | 202 | 12.46 | 269 | 13.3 | 259 | 14.14 | 258 | 14.98 | 253 |
| 11.64 | 246 | 12.48 | 214 | 13.32 | 242 | 14.16 | 267 | 15 | 266 |
| 11.66 | 218 | 12.5 | 282 | 13.34 | 260 | 14.18 | 274 | 15.02 | 254 |
| 11.68 | 218 | 12.52 | 236 | 13.36 | 281 | 14.2 | 261 | 15.04 | 275 |
| 11.7 | 239 | 12.54 | 217 | 13.38 | 272 | 14.22 | 260 | 15.06 | 246 |
| 11.72 | 227 | 12.56 | 264 | 13.4 | 248 | 14.24 | 287 | 15.08 | 260 |
| 11.74 | 239 | 12.58 | 248 | 13.42 | 294 | 14.26 | 266 | 15.1 | 276 |
| 11.76 | 212 | 12.6 | 249 | 13.44 | 285 | 14.28 | 266 | 15.12 | 258 |
| 11.78 | 246 | 12.62 | 256 | 13.46 | 287 | 14.3 | 267 | 15.14 | 266 |
| 11.8 | 246 | 12.64 | 251 | 13.48 | 267 | 14.32 | 238 | 15.16 | 237 |
| 11.82 | 246 | 12.66 | 245 | 13.5 | 274 | 14.34 | 257 | 15.18 | 251 |
| 11.84 | 234 | 12.68 | 260 | 13.52 | 321 | 14.36 | 236 | 15.2 | 291 |
| 11.86 | 233 | 12.7 | 243 | 13.54 | 327 | 14.38 | 270 | 15.22 | 250 |
| 11.88 | 222 | 12.72 | 271 | 13.56 | 400 | 14.4 | 267 | 15.24 | 254 |
| 11.9 | 236 | 12.74 | 248 | 13.58 | 448 | 14.42 | 255 | 15.26 | 252 |
| 11.92 | 228 | 12.76 | 254 | 13.6 | 593 | 14.44 | 269 | 15.28 | 274 |
| 11.94 | 248 | 12.78 | 229 | 13.62 | 678 | 14.46 | 287 | 15.3 | 264 |
| 11.96 | 236 | 12.8 | 253 | 13.64 | 671 | 14.48 | 289 | 15.32 | 267 |
| 11.98 | 224 | 12.82 | 276 | 13.66 | 573 | 14.5 | 271 | 15.34 | 246 |
| 12 | 210 | 12.84 | 230 | 13.68 | 434 | 14.52 | 280 | 15.36 | 257 |
| 12.02 | 224 | 12.86 | 269 | 13.7 | 316 | 14.54 | 280 | 15.38 | 286 |
| 12.04 | 237 | 12.88 | 259 | 13.72 | 265 | 14.56 | 283 | 15.4 | 256 |
| 12.06 | 254 | 12.9 | 242 | 13.74 | 303 | 14.58 | 242 | 15.42 | 243 |
| 12.08 | 221 | 12.92 | 244 | 13.76 | 285 | 14.6 | 245 | 15.44 | 254 |
| 12.1 | 223 | 12.94 | 245 | 13.78 | 253 | 14.62 | 240 | 15.46 | 238 |
| 12.12 | 239 | 12.96 | 264 | 13.8 | 269 | 14.64 | 268 | 15.48 | 278 |
| 12.14 | 241 | 12.98 | 256 | 13.82 | 281 | 14.66 | 250 | 15.5 | 235 |
| 12.16 | 259 | 13 | 232 | 13.84 | 297 | 14.68 | 267 | 15.52 | 241 |
| 12.18 | 236 | 13.02 | 266 | 13.86 | 311 | 14.7 | 256 | 15.54 | 247 |
| 12.2 | 251 | 13.04 | 213 | 13.88 | 314 | 14.72 | 232 | 15.56 | 245 |
| 12.22 | 222 | 13.06 | 271 | 13.9 | 273 | 14.74 | 274 | 15.58 | 264 |
| 15.6 | 272 | 16.44 | 521 | 17.28 | 261 | 18.12 | 236 | 18.96 | 612 |
| 15.62 | 243 | 16.46 | 506 | 17.3 | 233 | 18.14 | 250 | 18.98 | 614 |
| 15.64 | 264 | 16.48 | 417 | 17.32 | 251 | 18.16 | 252 | 19 | 532 |
| 15.66 | 253 | 16.5 | 348 | 17.34 | 244 | 18.18 | 229 | 19.02 | 431 |
| 15.68 | 253 | 16.52 | 296 | 17.36 | 237 | 18.2 | 246 | 19.04 | 356 |
| 15.7 | 249 | 16.54 | 263 | 17.38 | 234 | 18.22 | 248 | 19.06 | 284 |
| 15.72 | 249 | 16.56 | 254 | 17.4 | 256 | 18.24 | 251 | 19.08 | 295 |
| 15.74 | 257 | 16.58 | 221 | 17.42 | 250 | 18.26 | 245 | 19.1 | 238 |
| 15.76 | 263 | 16.6 | 245 | 17.44 | 267 | 18.28 | 224 | 19.12 | 271 |
| 15.78 | 248 | 16.62 | 243 | 17.46 | 263 | 18.3 | 257 | 19.14 | 214 |
| 15.8 | 239 | 16.64 | 246 | 17.48 | 255 | 18.32 | 244 | 19.16 | 229 |
| 15.82 | 256 | 16.66 | 250 | 17.5 | 281 | 18.34 | 231 | 19.18 | 275 |
| 15.84 | 264 | 16.68 | 254 | 17.52 | 265 | 18.36 | 259 | 19.2 | 259 |
| 15.86 | 257 | 16.7 | 248 | 17.54 | 270 | 18.38 | 239 | 19.22 | 236 |
| 15.88 | 262 | 16.72 | 224 | 17.56 | 295 | 18.4 | 261 | 19.24 | 247 |
| 15.9 | 247 | 16.74 | 266 | 17.58 | 300 | 18.42 | 245 | 19.26 | 229 |
| 15.92 | 238 | 16.76 | 218 | 17.6 | 330 | 18.44 | 230 | 19.28 | 232 |
| 15.94 | 245 | 16.78 | 245 | 17.62 | 440 | 18.46 | 265 | 19.3 | 269 |
| 15.96 | 234 | 16.8 | 256 | 17.64 | 519 | 18.48 | 247 | 19.32 | 256 |
| 15.98 | 246 | 16.82 | 214 | 17.66 | 453 | 18.5 | 233 | 19.34 | 212 |
| 16 | 236 | 16.84 | 265 | 17.68 | 401 | 18.52 | 213 | 19.36 | 241 |
| 16.02 | 237 | 16.86 | 237 | 17.7 | 326 | 18.54 | 227 | 19.38 | 220 |
| 16.04 | 274 | 16.88 | 217 | 17.72 | 297 | 18.56 | 233 | 19.4 | 240 |
| 16.06 | 272 | 16.9 | 243 | 17.74 | 249 | 18.58 | 249 | 19.42 | 265 |
| 16.08 | 278 | 16.92 | 267 | 17.76 | 283 | 18.6 | 259 | 19.44 | 269 |
| 16.1 | 265 | 16.94 | 232 | 17.78 | 279 | 18.62 | 245 | 19.46 | 229 |
| 16.12 | 232 | 16.96 | 253 | 17.8 | 297 | 18.64 | 234 | 19.48 | 242 |
| 16.14 | 214 | 16.98 | 262 | 17.82 | 285 | 18.66 | 226 | 19.5 | 234 |
| 16.16 | 264 | 17 | 254 | 17.84 | 267 | 18.68 | 220 | 19.52 | 234 |
| 16.18 | 249 | 17.02 | 249 | 17.86 | 279 | 18.7 | 247 | 19.54 | 276 |
| 16.2 | 251 | 17.04 | 229 | 17.88 | 303 | 18.72 | 293 | 19.56 | 224 |
| 16.22 | 225 | 17.06 | 252 | 17.9 | 285 | 18.74 | 228 | 19.58 | 258 |
| 16.24 | 269 | 17.08 | 228 | 17.92 | 325 | 18.76 | 284 | 19.6 | 254 |
| 16.26 | 276 | 17.1 | 226 | 17.94 | 347 | 18.78 | 234 | 19.62 | 257 |
| 16.28 | 260 | 17.12 | 200 | 17.96 | 454 | 18.8 | 248 | 19.64 | 265 |
| 16.3 | 236 | 17.14 | 253 | 17.98 | 512 | 18.82 | 260 | 19.66 | 232 |
| 16.32 | 256 | 17.16 | 247 | 18 | 481 | 18.84 | 277 | 19.68 | 246 |
| 16.34 | 255 | 17.18 | 234 | 18.02 | 421 | 18.86 | 291 | 19.7 | 234 |
| 16.36 | 252 | 17.2 | 252 | 18.04 | 329 | 18.88 | 285 | 19.72 | 248 |
| 16.38 | 310 | 17.22 | 248 | 18.06 | 308 | 18.9 | 311 | 19.74 | 249 |
| 16.4 | 358 | 17.24 | 255 | 18.08 | 242 | 18.92 | 311 | 19.76 | 219 |
| 16.42 | 487 | 17.26 | 236 | 18.1 | 219 | 18.94 | 449 | 19.78 | 218 |
| 19.8 | 233 | 20.64 | 233 | 21.48 | 264 | 22.32 | 262 | 23.16 | 258 |
| 19.82 | 245 | 20.66 | 243 | 21.5 | 252 | 22.34 | 253 | 23.18 | 231 |
| 19.84 | 228 | 20.68 | 244 | 21.52 | 242 | 22.36 | 258 | 23.2 | 262 |
| 19.86 | 223 | 20.7 | 238 | 21.54 | 247 | 22.38 | 249 | 23.22 | 251 |
| 19.88 | 267 | 20.72 | 256 | 21.56 | 254 | 22.4 | 253 | 23.24 | 227 |
| 19.9 | 212 | 20.74 | 253 | 21.58 | 240 | 22.42 | 258 | 23.26 | 235 |
| 19.92 | 226 | 20.76 | 255 | 21.6 | 249 | 22.44 | 261 | 23.28 | 247 |
| 19.94 | 224 | 20.78 | 261 | 21.62 | 222 | 22.46 | 295 | 23.3 | 245 |
| 19.96 | 223 | 20.8 | 231 | 21.64 | 245 | 22.48 | 321 | 23.32 | 250 |
| 19.98 | 245 | 20.82 | 251 | 21.66 | 270 | 22.5 | 368 | 23.34 | 238 |
| 20 | 265 | 20.84 | 263 | 21.68 | 232 | 22.52 | 364 | 23.36 | 262 |
| 20.02 | 247 | 20.86 | 295 | 21.7 | 227 | 22.54 | 317 | 23.38 | 266 |
| 20.04 | 252 | 20.88 | 267 | 21.72 | 241 | 22.56 | 283 | 2.34 | 236 |
| 20.06 | 260 | 20.9 | 261 | 21.74 | 255 | 22.58 | 300 | 23.42 | 245 |
| 20.08 | 249 | 20.92 | 255 | 21.76 | 254 | 22.6 | 285 | 23.44 | 244 |
| 20.1 | 243 | 20.94 | 271 | 21.78 | 232 | 22.62 | 284 | 23.46 | 243 |
| 20.12 | 233 | 20.96 | 245 | 21.8 | 245 | 22.64 | 264 | 23.48 | 265 |
| 20.14 | 247 | 20.98 | 263 | 21.82 | 256 | 22.66 | 271 | 23.5 | 285 |
| 20.16 | 250 | 21 | 255 | 21.84 | 244 | 22.68 | 278 | 23.52 | 245 |
| 20.18 | 265 | 21.02 | 242 | 21.86 | 214 | 22.7 | 258 | 23.54 | 277 |
| 20.2 | 277 | 21.04 | 273 | 21.88 | 243 | 22.72 | 282 | 23.56 | 264 |
| 20.22 | 265 | 21.06 | 261 | 21.9 | 266 | 22.74 | 252 | 23.58 | 273 |
| 20.24 | 268 | 21.08 | 249 | 21.92 | 238 | 22.76 | 223 | 23.6 | 259 |
| 20.26 | 218 | 21.1 | 272 | 21.94 | 251 | 22.78 | 277 | 23.62 | 296 |
| 20.28 | 258 | 21.12 | 252 | 21.96 | 231 | 22.8 | 236 | 23.64 | 255 |
| 20.3 | 262 | 21.14 | 271 | 21.98 | 255 | 22.82 | 252 | 23.66 | 252 |
| 20.32 | 245 | 21.16 | 241 | 22 | 259 | 22.84 | 238 | 23.68 | 240 |
| 20.34 | 247 | 21.18 | 277 | 22.02 | 237 | 22.86 | 278 | 23.7 | 220 |
| 20.36 | 256 | 21.2 | 241 | 22.04 | 285 | 22.88 | 245 | 23.72 | 251 |
| 20.38 | 250 | 21.22 | 248 | 22.06 | 230 | 22.9 | 235 | 23.74 | 262 |
| 20.4 | 209 | 21.24 | 225 | 22.08 | 260 | 22.92 | 257 | 23.76 | 224 |
| 20.42 | 247 | 21.26 | 245 | 22.1 | 252 | 22.94 | 238 | 23.78 | 241 |
| 20.44 | 247 | 21.28 | 244 | 22.12 | 245 | 22.96 | 251 | 23.8 | 239 |
| 20.46 | 236 | 21.3 | 259 | 22.14 | 254 | 22.98 | 269 | 23.82 | 237 |
| 20.48 | 264 | 21.32 | 258 | 22.16 | 253 | 23 | 226 | 23.84 | 238 |
| 20.5 | 250 | 21.34 | 267 | 22.18 | 282 | 23.02 | 237 | 23.86 | 266 |
| 20.52 | 257 | 21.36 | 272 | 22.2 | 246 | 23.04 | 239 | 23.88 | 240 |
| 20.54 | 235 | 21.38 | 272 | 22.22 | 296 | 23.06 | 238 | 23.9 | 256 |
| 20.56 | 255 | 21.4 | 288 | 22.24 | 303 | 23.08 | 228 | 23.92 | 264 |
| 20.58 | 231 | 21.42 | 299 | 22.26 | 247 | 23.1 | 245 | 23.94 | 244 |
| 20.6 | 208 | 21.44 | 237 | 22.28 | 261 | 23.12 | 252 | 23.96 | 224 |
| 20.62 | 229 | 21.46 | 249 | 22.3 | 284 | 23.14 | 254 | 23.98 | 241 |
| 24 | 225 | 24.84 | 249 | 25.68 | 258 | 26.52 | 236 | 27.36 | 225 |
| 24.02 | 251 | 24.86 | 233 | 25.7 | 248 | 26.54 | 237 | 27.38 | 212 |
| 24.04 | 272 | 24.88 | 242 | 25.72 | 275 | 26.56 | 233 | 27.4 | 224 |
| 24.06 | 281 | 24.9 | 251 | 25.74 | 271 | 26.58 | 251 | 27.42 | 244 |
| 24.08 | 232 | 24.92 | 245 | 25.76 | 286 | 26.6 | 227 | 27.44 | 207 |
| 24.1 | 264 | 24.94 | 260 | 25.78 | 263 | 26.62 | 212 | 27.46 | 237 |
| 24.12 | 256 | 24.96 | 243 | 25.8 | 274 | 26.64 | 235 | 27.48 | 235 |
| 24.14 | 261 | 24.98 | 251 | 25.82 | 263 | 26.66 | 233 | 27.5 | 271 |
| 24.16 | 245 | 25 | 256 | 25.84 | 239 | 26.68 | 226 | 27.52 | 228 |
| 24.18 | 277 | 25.02 | 257 | 25.86 | 236 | 26.7 | 247 | 27.54 | 233 |
| 24.2 | 235 | 25.04 | 268 | 25.88 | 223 | 26.72 | 230 | 27.56 | 243 |
| 24.22 | 238 | 25.06 | 279 | 25.9 | 213 | 26.74 | 228 | 27.58 | 224 |
| 24.24 | 250 | 25.08 | 276 | 25.92 | 245 | 26.76 | 231 | 27.6 | 225 |
| 24.26 | 238 | 25.1 | 275 | 25.94 | 246 | 26.78 | 239 | 27.62 | 217 |
| 24.28 | 261 | 25.12 | 251 | 25.96 | 223 | 26.8 | 231 | 27.64 | 212 |
| 24.3 | 251 | 25.14 | 262 | 25.98 | 238 | 26.82 | 257 | 27.66 | 223 |
| 24.32 | 236 | 25.16 | 266 | 26 | 227 | 26.84 | 273 | 27.68 | 223 |
| 24.34 | 246 | 25.18 | 243 | 26.02 | 197 | 26.86 | 239 | 27.7 | 236 |
| 24.36 | 234 | 25.2 | 234 | 26.04 | 247 | 26.88 | 284 | 27.72 | 200 |
| 24.38 | 237 | 25.22 | 226 | 26.06 | 232 | 26.9 | 269 | 27.74 | 231 |
| 24.4 | 247 | 25.24 | 261 | 26.08 | 225 | 26.92 | 281 | 27.76 | 219 |
| 24.42 | 221 | 25.26 | 255 | 26.1 | 215 | 26.94 | 279 | 27.78 | 224 |
| 24.44 | 228 | 25.28 | 238 | 26.12 | 244 | 26.96 | 252 | 27.8 | 231 |
| 24.46 | 232 | 25.3 | 239 | 26.14 | 227 | 26.98 | 259 | 27.82 | 262 |
| 24.48 | 273 | 25.32 | 204 | 26.16 | 231 | 27 | 270 | 27.84 | 229 |
| 24.5 | 251 | 25.34 | 243 | 26.18 | 224 | 27.02 | 268 | 27.86 | 247 |
| 24.52 | 219 | 25.36 | 249 | 26.2 | 233 | 27.04 | 282 | 27.88 | 272 |
| 24.54 | 241 | 25.38 | 251 | 26.22 | 233 | 27.06 | 323 | 27.9 | 361 |
| 24.56 | 226 | 25.4 | 271 | 26.24 | 236 | 27.08 | 263 | 27.92 | 404 |
| 24.58 | 244 | 25.42 | 231 | 26.26 | 254 | 27.1 | 251 | 27.94 | 377 |
| 24.6 | 269 | 25.44 | 232 | 26.28 | 232 | 27.12 | 260 | 27.96 | 333 |
| 24.62 | 258 | 25.46 | 242 | 26.3 | 210 | 27.14 | 229 | 27.98 | 306 |
| 24.64 | 238 | 25.48 | 244 | 26.32 | 219 | 27.16 | 250 | 28 | 314 |
| 24.66 | 234 | 25.5 | 244 | 26.34 | 204 | 27.18 | 238 | 28.02 | 263 |
| 24.68 | 262 | 25.52 | 241 | 26.36 | 250 | 27.2 | 227 | 28.04 | 227 |
| 24.7 | 232 | 25.54 | 210 | 26.38 | 224 | 27.22 | 206 | 28.06 | 233 |
| 24.72 | 249 | 25.56 | 247 | 26.4 | 253 | 27.24 | 218 | 28.08 | 209 |
| 24.74 | 240 | 25.58 | 218 | 26.42 | 226 | 27.26 | 215 | 28.1 | 223 |
| 24.76 | 216 | 25.6 | 244 | 26.44 | 234 | 27.28 | 221 | 28.12 | 216 |
| 24.78 | 225 | 25.62 | 242 | 26.46 | 203 | 27.3 | 201 | 28.14 | 192 |
| 24.8 | 214 | 25.64 | 258 | 26.48 | 234 | 27.32 | 221 | 28.16 | 207 |
| 24.82 | 257 | 25.66 | 240 | 26.5 | 246 | 27.34 | 200 | 28.18 | 207 |
| 28.2 | 198 | 29.04 | 254 | 29.88 | 199 | 30.72 | 186 | 31.56 | 187 |
| 28.22 | 229 | 29.06 | 254 | 29.9 | 187 | 30.74 | 192 | 31.58 | 199 |
| 28.24 | 190 | 29.08 | 243 | 29.92 | 213 | 30.76 | 184 | 31.6 | 192 |
| 28.26 | 237 | 29.1 | 256 | 29.94 | 176 | 30.78 | 190 | 31.62 | 166 |
| 28.28 | 242 | 29.12 | 216 | 29.96 | 223 | 30.8 | 178 | 31.64 | 170 |
| 28.3 | 246 | 29.14 | 235 | 29.98 | 202 | 30.82 | 178 | 31.66 | 178 |
| 28.32 | 208 | 29.16 | 224 | 30 | 192 | 30.84 | 167 | 31.68 | 194 |
| 28.34 | 197 | 29.18 | 223 | 30.02 | 183 | 30.86 | 181 | 31.7 | 177 |
| 28.36 | 243 | 29.2 | 204 | 30.04 | 200 | 30.88 | 168 | 31.72 | 171 |
| 28.38 | 205 | 29.22 | 199 | 30.06 | 199 | 30.9 | 169 | 31.74 | 166 |
| 28.4 | 208 | 29.24 | 261 | 30.08 | 198 | 30.92 | 177 | 31.76 | 168 |
| 28.42 | 218 | 29.26 | 233 | 30.1 | 164 | 30.94 | 174 | 31.78 | 179 |
| 28.44 | 203 | 29.28 | 257 | 30.12 | 205 | 30.96 | 201 | 31.8 | 168 |
| 28.46 | 217 | 29.3 | 265 | 30.14 | 186 | 30.98 | 187 | 31.82 | 184 |
| 28.48 | 218 | 29.32 | 280 | 30.16 | 178 | 31 | 167 | 31.84 | 169 |
| 28.5 | 237 | 29.34 | 276 | 30.18 | 188 | 31.02 | 190 | 31.86 | 166 |
| 28.52 | 255 | 29.36 | 263 | 30.2 | 206 | 31.04 | 193 | 31.88 | 163 |
| 28.54 | 242 | 29.38 | 227 | 30.22 | 201 | 31.06 | 183 | 31.9 | 175 |
| 28.56 | 226 | 29.4 | 250 | 30.24 | 205 | 31.08 | 170 | 31.92 | 195 |
| 28.58 | 220 | 29.42 | 199 | 30.26 | 208 | 31.1 | 192 | 31.94 | 155 |
| 28.6 | 196 | 29.44 | 233 | 30.28 | 186 | 31.12 | 166 | 31.96 | 180 |
| 28.62 | 231 | 29.46 | 211 | 30.3 | 195 | 31.14 | 175 | 31.98 | 157 |
| 28.64 | 246 | 29.48 | 234 | 30.32 | 200 | 31.16 | 175 | 32 | 169 |
| 28.66 | 207 | 29.5 | 218 | 30.34 | 204 | 31.18 | 204 | 32.02 | 167 |
| 28.68 | 192 | 29.52 | 211 | 30.36 | 169 | 31.2 | 189 | 32.04 | 160 |
| 28.7 | 206 | 29.54 | 218 | 30.38 | 204 | 31.22 | 170 | 32.06 | 190 |
| 28.72 | 213 | 29.56 | 199 | 30.4 | 213 | 31.24 | 191 | 32.08 | 184 |
| 28.74 | 234 | 29.58 | 193 | 30.42 | 184 | 31.26 | 170 | 32.1 | 176 |
| 28.76 | 193 | 29.6 | 195 | 30.44 | 187 | 31.28 | 203 | 32.12 | 200 |
| 28.78 | 196 | 29.62 | 203 | 30.46 | 181 | 31.3 | 192 | 32.14 | 204 |
| 28.8 | 220 | 29.64 | 185 | 30.48 | 185 | 31.32 | 223 | 32.16 | 194 |
| 28.82 | 222 | 29.66 | 187 | 30.5 | 167 | 31.34 | 185 | 32.18 | 166 |
| 28.84 | 187 | 29.68 | 185 | 30.52 | 180 | 31.36 | 201 | 32.2 | 158 |
| 28.86 | 195 | 29.7 | 200 | 30.54 | 193 | 31.38 | 182 | 32.22 | 173 |
| 28.88 | 226 | 29.72 | 185 | 30.56 | 195 | 31.4 | 185 | 32.24 | 178 |
| 28.9 | 209 | 29.74 | 221 | 30.58 | 185 | 31.42 | 182 | 32.26 | 179 |
| 28.92 | 212 | 29.76 | 221 | 30.6 | 191 | 31.44 | 189 | 32.28 | 176 |
| 28.94 | 201 | 29.78 | 209 | 30.62 | 176 | 31.46 | 173 | 32.3 | 146 |
| 28.96 | 244 | 29.8 | 211 | 30.64 | 190 | 31.48 | 203 | 32.32 | 166 |
| 28.98 | 230 | 29.82 | 189 | 30.66 | 177 | 31.5 | 152 | 32.34 | 167 |
| 29 | 216 | 29.84 | 219 | 30.68 | 169 | 31.52 | 156 | 32.36 | 169 |
| 29.02 | 267 | 29.86 | 195 | 30.7 | 186 | 31.54 | 174 | 32.38 | 181 |
| 32.4 | 162 | 33.24 | 156 | 34.08 | 175 | 34.92 | 172 | | |
| 32.42 | 192 | 33.26 | 139 | 34.1 | 157 | 34.94 | 138 | | |
| 32.44 | 165 | 33.28 | 171 | 34.12 | 163 | 34.96 | 139 | | |
| 32.46 | 163 | 33.3 | 160 | 34.14 | 149 | 34.98 | 167 | | |
| 32.48 | 175 | 33.32 | 171 | 34.16 | 160 | 35 | 136 | | |
| 32.5 | 172 | 33.34 | 167 | 34.18 | 159 | | | | |
| 32.52 | 170 | 33.36 | 161 | 34.2 | 140 | | | | |
| 32.54 | 175 | 33.38 | 157 | 34.22 | 145 | | | | |
| 32.56 | 170 | 33.4 | 169 | 34.24 | 151 | | | | |
| 32.58 | 177 | 33.42 | 166 | 34.26 | 155 | | | | |
| 32.6 | 210 | 33.44 | 149 | 34.28 | 126 | | | | |
| 32.62 | 167 | 33.46 | 167 | 34.3 | 143 | | | | |
| 32.64 | 180 | 33.48 | 156 | 34.32 | 152 | | | | |
| 32.66 | 169 | 33.5 | 173 | 34.34 | 178 | | | | |
| 32.68 | 170 | 33.52 | 162 | 34.36 | 163 | | | | |
| 32.7 | 158 | 33.54 | 139 | 34.38 | 146 | | | | |
| 32.72 | 171 | 33.56 | 161 | 34.4 | 146 | | | | |
| 32.74 | 164 | 33.58 | 166 | 34.42 | 151 | | | | |
| 32.76 | 165 | 33.6 | 166 | 34.44 | 156 | | | | |
| 32.78 | 191 | 33.62 | 163 | 34.46 | 153 | | | | |
| 32.8 | 163 | 33.64 | 158 | 34.48 | 153 | | | | |
| 32.82 | 162 | 33.66 | 162 | 34.5 | 140 | | | | |
| 32.84 | 203 | 33.68 | 147 | 34.52 | 149 | | | | |
| 32.86 | 167 | 33.7 | 161 | 34.54 | 146 | | | | |
| 32.88 | 180 | 33.72 | 171 | 34.56 | 152 | | | | |
| 32.9 | 170 | 33.74 | 157 | 34.58 | 166 | | | | |
| 32.92 | 171 | 33.76 | 162 | 34.6 | 159 | | | | |
| 32.94 | 187 | 33.78 | 151 | 34.62 | 154 | | | | |
| 32.96 | 171 | 33.8 | 156 | 34.64 | 138 | | | | |
| 32.98 | 177 | 33.82 | 164 | 34.66 | 127 | | | | |
| 33 | 171 | 33.84 | 151 | 34.68 | 155 | | | | |
| 33.02 | 207 | 33.86 | 150 | 34.7 | 151 | | | | |
| 33.04 | 154 | 33.88 | 158 | 34.72 | 143 | | | | |
| 33.06 | 173 | 33.9 | 156 | 34.74 | 143 | | | | |
| 33.08 | 170 | 33.92 | 148 | 34.76 | 137 | | | | |
| 33.1 | 154 | 33.94 | 164 | 34.78 | 120 | | | | |
| 33.12 | 181 | 33.96 | 168 | 34.8 | 174 | | | | |
| 33.14 | 175 | 33.98 | 158 | 34.82 | 161 | | | | |
| 33.16 | 174 | 34 | 166 | 34.84 | 123 | | | | |
| 33.18 | 151 | 34.02 | 168 | 34.86 | 165 | | | | |
| 33.2 | 182 | 34.04 | 146 | 34.88 | 157 | | | | |
| 33.22 | 197 | 34.06 | 178 | 34.9 | 169 | | | | |

FIG. 12

| 2Theta | 106 | 2Theta | 106 | 2Theta | 106 | 2Theta | 106 | 2Theta | 106 |
|---|---|---|---|---|---|---|---|---|---|
| 3 | 128 | 3.84 | 102 | 4.68 | 108 | 5.52 | 135 | 6.36 | 153 |
| 3.02 | 109 | 3.86 | 89 | 4.7 | 106 | 5.54 | 104 | 6.38 | 158 |
| 3.04 | 102 | 3.88 | 96 | 4.72 | 128 | 5.56 | 119 | 6.4 | 142 |
| 3.06 | 107 | 3.9 | 105 | 4.74 | 103 | 5.58 | 146 | 6.42 | 139 |
| 3.08 | 102 | 3.92 | 97 | 4.76 | 104 | 5.6 | 141 | 6.44 | 152 |
| 3.1 | 116 | 3.94 | 109 | 4.78 | 101 | 5.62 | 144 | 6.46 | 146 |
| 3.12 | 113 | 3.96 | 110 | 4.8 | 111 | 5.64 | 121 | 6.48 | 159 |
| 3.14 | 111 | 3.98 | 95 | 4.82 | 114 | 5.66 | 117 | 6.5 | 176 |
| 3.16 | 123 | 4 | 89 | 4.84 | 115 | 5.68 | 110 | 6.52 | 138 |
| 3.18 | 113 | 4.02 | 104 | 4.86 | 113 | 5.7 | 134 | 6.54 | 164 |
| 3.2 | 110 | 4.04 | 105 | 4.88 | 135 | 5.72 | 134 | 6.56 | 155 |
| 3.22 | 118 | 4.06 | 103 | 4.9 | 113 | 5.74 | 133 | 6.58 | 159 |
| 3.24 | 98 | 4.08 | 112 | 4.92 | 104 | 5.76 | 120 | 6.6 | 124 |
| 3.26 | 94 | 4.1 | 111 | 4.94 | 115 | 5.78 | 137 | 6.62 | 136 |
| 3.28 | 103 | 4.12 | 103 | 4.96 | 134 | 5.8 | 136 | 6.64 | 153 |
| 3.3 | 113 | 4.14 | 101 | 4.98 | 119 | 5.82 | 150 | 6.66 | 167 |
| 3.32 | 102 | 4.16 | 114 | 5 | 117 | 5.84 | 126 | 6.68 | 158 |
| 3.34 | 92 | 4.18 | 108 | 5.02 | 120 | 5.86 | 133 | 6.7 | 171 |
| 3.36 | 100 | 4.2 | 106 | 5.04 | 109 | 5.88 | 142 | 6.72 | 178 |
| 3.38 | 126 | 4.22 | 110 | 5.06 | 151 | 5.9 | 136 | 6.74 | 161 |
| 3.4 | 87 | 4.24 | 110 | 5.08 | 116 | 5.92 | 137 | 6.76 | 146 |
| 3.42 | 97 | 4.26 | 101 | 5.1 | 86 | 5.94 | 140 | 6.78 | 145 |
| 3.44 | 108 | 4.28 | 102 | 5.12 | 133 | 5.96 | 135 | 6.8 | 155 |
| 3.46 | 97 | 4.3 | 112 | 5.14 | 130 | 5.98 | 140 | 6.82 | 142 |
| 3.48 | 104 | 4.32 | 111 | 5.16 | 120 | 6 | 133 | 6.84 | 162 |
| 3.5 | 114 | 4.34 | 116 | 5.18 | 120 | 6.02 | 134 | 6.86 | 187 |
| 3.52 | 110 | 4.36 | 100 | 5.2 | 122 | 6.04 | 149 | 6.88 | 185 |
| 3.54 | 100 | 4.38 | 94 | 5.22 | 124 | 6.06 | 134 | 6.9 | 212 |
| 3.56 | 120 | 4.4 | 104 | 5.24 | 106 | 6.08 | 126 | 6.92 | 276 |
| 3.58 | 101 | 4.42 | 106 | 5.26 | 114 | 6.1 | 142 | 6.94 | 262 |
| 3.6 | 106 | 4.44 | 100 | 5.28 | 131 | 6.12 | 148 | 6.96 | 242 |
| 3.62 | 140 | 4.46 | 100 | 5.3 | 122 | 6.14 | 136 | 6.98 | 198 |
| 3.64 | 95 | 4.48 | 98 | 5.32 | 128 | 6.16 | 145 | 7 | 157 |
| 3.66 | 105 | 4.5 | 102 | 5.34 | 123 | 6.18 | 131 | 7.02 | 156 |
| 3.68 | 103 | 4.52 | 114 | 5.36 | 135 | 6.2 | 143 | 7.04 | 151 |
| 3.7 | 95 | 4.54 | 91 | 5.38 | 129 | 6.22 | 155 | 7.06 | 160 |
| 3.72 | 117 | 4.56 | 123 | 5.4 | 117 | 6.24 | 135 | 7.08 | 191 |
| 3.74 | 106 | 4.58 | 103 | 5.42 | 130 | 6.26 | 180 | 7.1 | 171 |
| 3.76 | 102 | 4.6 | 109 | 5.44 | 123 | 6.28 | 146 | 7.12 | 138 |
| 3.78 | 96 | 4.62 | 96 | 5.46 | 134 | 6.3 | 147 | 7.14 | 154 |
| 3.8 | 122 | 4.64 | 93 | 5.48 | 140 | 6.32 | 116 | 7.16 | 141 |
| 3.82 | 112 | 4.66 | 127 | 5.5 | 139 | 6.34 | 145 | 7.18 | 145 |
| 7.2 | 149 | 8.04 | 162 | 8.88 | 171 | 9.72 | 199 | 10.56 | 204 |
| 7.22 | 167 | 8.06 | 151 | 8.9 | 195 | 9.74 | 225 | 10.58 | 218 |
| 7.24 | 169 | 8.08 | 188 | 8.92 | 186 | 9.76 | 194 | 10.6 | 219 |
| 7.26 | 132 | 8.1 | 211 | 8.94 | 183 | 9.78 | 207 | 10.62 | 219 |
| 7.28 | 125 | 8.12 | 206 | 8.96 | 193 | 9.8 | 192 | 10.64 | 237 |
| 7.3 | 158 | 8.14 | 233 | 8.98 | 175 | 9.82 | 216 | 10.66 | 228 |
| 7.32 | 161 | 8.16 | 221 | 9 | 196 | 9.84 | 216 | 10.68 | 223 |
| 7.34 | 146 | 8.18 | 167 | 9.02 | 175 | 9.86 | 210 | 10.7 | 197 |
| 7.36 | 153 | 8.2 | 195 | 9.04 | 184 | 9.88 | 196 | 10.72 | 222 |
| 7.38 | 168 | 8.22 | 188 | 9.06 | 211 | 9.9 | 220 | 10.74 | 232 |
| 7.4 | 172 | 8.24 | 175 | 9.08 | 181 | 9.92 | 206 | 10.76 | 201 |
| 7.42 | 141 | 8.26 | 172 | 9.1 | 171 | 9.94 | 211 | 10.78 | 210 |
| 7.44 | 146 | 8.28 | 166 | 9.12 | 179 | 9.96 | 210 | 10.8 | 231 |
| 7.46 | 204 | 8.3 | 172 | 9.14 | 186 | 9.98 | 201 | 10.82 | 214 |
| 7.48 | 159 | 8.32 | 179 | 9.16 | 200 | 10 | 181 | 10.84 | 236 |
| 7.5 | 150 | 8.34 | 163 | 9.18 | 201 | 10.02 | 207 | 10.86 | 253 |
| 7.52 | 162 | 8.36 | 154 | 9.2 | 209 | 10.04 | 212 | 10.88 | 193 |
| 7.54 | 156 | 8.38 | 162 | 9.22 | 193 | 10.06 | 219 | 10.9 | 210 |
| 7.56 | 165 | 8.4 | 184 | 9.24 | 191 | 10.08 | 222 | 10.92 | 228 |
| 7.58 | 184 | 8.42 | 172 | 9.26 | 182 | 10.1 | 194 | 10.94 | 231 |
| 7.6 | 166 | 8.44 | 168 | 9.28 | 205 | 10.12 | 198 | 10.96 | 252 |
| 7.62 | 149 | 8.46 | 171 | 9.3 | 192 | 10.14 | 194 | 10.98 | 247 |
| 7.64 | 158 | 8.48 | 188 | 9.32 | 193 | 10.16 | 211 | 11 | 261 |
| 7.66 | 185 | 8.5 | 151 | 9.34 | 182 | 10.18 | 213 | 11.02 | 265 |
| 7.68 | 175 | 8.52 | 178 | 9.36 | 218 | 10.2 | 209 | 11.04 | 289 |
| 7.7 | 161 | 8.54 | 165 | 9.38 | 189 | 10.22 | 205 | 11.06 | 320 |
| 7.72 | 159 | 8.56 | 191 | 9.4 | 183 | 10.24 | 212 | 11.08 | 268 |
| 7.74 | 145 | 8.58 | 159 | 9.42 | 187 | 10.26 | 218 | 11.1 | 330 |
| 7.76 | 155 | 8.6 | 174 | 9.44 | 187 | 10.28 | 197 | 11.12 | 304 |
| 7.78 | 170 | 8.62 | 151 | 9.46 | 185 | 10.3 | 209 | 11.14 | 343 |
| 7.8 | 187 | 8.64 | 193 | 9.48 | 223 | 10.32 | 172 | 11.16 | 384 |
| 7.82 | 169 | 8.66 | 187 | 9.5 | 223 | 10.34 | 242 | 11.18 | 478 |
| 7.84 | 167 | 8.68 | 196 | 9.52 | 180 | 10.36 | 203 | 11.2 | 686 |
| 7.86 | 163 | 8.7 | 187 | 9.54 | 202 | 10.38 | 229 | 11.22 | 716 |
| 7.88 | 183 | 8.72 | 151 | 9.56 | 204 | 10.4 | 194 | 11.24 | 628 |
| 7.9 | 173 | 8.74 | 156 | 9.58 | 199 | 10.42 | 204 | 11.26 | 427 |
| 7.92 | 186 | 8.76 | 197 | 9.6 | 193 | 10.44 | 218 | 11.28 | 287 |
| 7.94 | 172 | 8.78 | 154 | 9.62 | 207 | 10.46 | 219 | 11.3 | 251 |
| 7.96 | 154 | 8.8 | 168 | 9.64 | 199 | 10.48 | 205 | 11.32 | 265 |
| 7.98 | 161 | 8.82 | 185 | 9.66 | 198 | 10.5 | 237 | 11.34 | 238 |
| 8 | 146 | 8.84 | 162 | 9.68 | 192 | 10.52 | 249 | 11.36 | 233 |
| 8.02 | 165 | 8.86 | 177 | 9.7 | 212 | 10.54 | 216 | 11.38 | 222 |
| 11.4 | 249 | 12.24 | 247 | 13.08 | 248 | 13.92 | 290 | 14.76 | 258 |
| 11.42 | 268 | 12.26 | 245 | 13.1 | 255 | 13.94 | 280 | 14.78 | 257 |
| 11.44 | 219 | 12.28 | 226 | 13.12 | 231 | 13.96 | 272 | 14.8 | 273 |
| 11.46 | 250 | 12.3 | 255 | 13.14 | 260 | 13.98 | 279 | 14.82 | 265 |
| 11.48 | 231 | 12.32 | 269 | 13.16 | 286 | 14 | 288 | 14.84 | 270 |
| 11.5 | 240 | 12.34 | 262 | 13.18 | 263 | 14.02 | 283 | 14.86 | 245 |
| 11.52 | 233 | 12.36 | 245 | 13.2 | 262 | 14.04 | 251 | 14.88 | 267 |
| 11.54 | 210 | 12.38 | 260 | 13.22 | 247 | 14.06 | 281 | 14.9 | 235 |
| 11.56 | 214 | 12.4 | 264 | 13.24 | 285 | 14.08 | 262 | 14.92 | 283 |
| 11.58 | 237 | 12.42 | 244 | 13.26 | 257 | 14.1 | 272 | 14.94 | 269 |
| 11.6 | 265 | 12.44 | 263 | 13.28 | 264 | 14.12 | 291 | 14.96 | 262 |
| 11.62 | 220 | 12.46 | 270 | 13.3 | 247 | 14.14 | 280 | 14.98 | 281 |
| 11.64 | 241 | 12.48 | 246 | 13.32 | 235 | 14.16 | 253 | 15 | 279 |
| 11.66 | 211 | 12.5 | 239 | 13.34 | 273 | 14.18 | 286 | 15.02 | 261 |
| 11.68 | 239 | 12.52 | 254 | 13.36 | 305 | 14.2 | 249 | 15.04 | 273 |
| 11.7 | 248 | 12.54 | 259 | 13.38 | 305 | 14.22 | 278 | 15.06 | 270 |
| 11.72 | 228 | 12.56 | 227 | 13.4 | 289 | 14.24 | 267 | 15.08 | 286 |
| 11.74 | 250 | 12.58 | 217 | 13.42 | 310 | 14.26 | 239 | 15.1 | 250 |
| 11.76 | 241 | 12.6 | 258 | 13.44 | 309 | 14.28 | 265 | 15.12 | 227 |
| 11.78 | 256 | 12.62 | 265 | 13.46 | 325 | 14.3 | 261 | 15.14 | 266 |
| 11.8 | 214 | 12.64 | 248 | 13.48 | 348 | 14.32 | 285 | 15.16 | 265 |
| 11.82 | 218 | 12.66 | 219 | 13.5 | 395 | 14.34 | 280 | 15.18 | 256 |
| 11.84 | 244 | 12.68 | 270 | 13.52 | 393 | 14.36 | 236 | 15.2 | 269 |
| 11.86 | 234 | 12.7 | 255 | 13.54 | 496 | 14.38 | 289 | 15.22 | 262 |
| 11.88 | 240 | 12.72 | 250 | 13.56 | 589 | 14.4 | 254 | 15.24 | 275 |
| 11.9 | 231 | 12.74 | 263 | 13.58 | 666 | 14.42 | 291 | 15.26 | 265 |
| 11.92 | 263 | 12.76 | 249 | 13.6 | 869 | 14.44 | 252 | 15.28 | 296 |
| 11.94 | 221 | 12.78 | 253 | 13.62 | 1176 | 14.46 | 272 | 15.3 | 244 |
| 11.96 | 241 | 12.8 | 251 | 13.64 | 1400 | 14.48 | 278 | 15.32 | 263 |
| 11.98 | 249 | 12.82 | 225 | 13.66 | 1353 | 14.5 | 286 | 15.34 | 245 |
| 12 | 256 | 12.84 | 263 | 13.68 | 978 | 14.52 | 282 | 15.36 | 261 |
| 12.02 | 215 | 12.86 | 242 | 13.7 | 718 | 14.54 | 332 | 15.38 | 231 |
| 12.04 | 249 | 12.88 | 258 | 13.72 | 439 | 14.56 | 260 | 15.4 | 259 |
| 12.06 | 267 | 12.9 | 296 | 13.74 | 360 | 14.58 | 289 | 15.42 | 241 |
| 12.08 | 246 | 12.92 | 228 | 13.76 | 292 | 14.6 | 280 | 15.44 | 315 |
| 12.1 | 244 | 12.94 | 247 | 13.78 | 291 | 14.62 | 282 | 15.46 | 279 |
| 12.12 | 230 | 12.96 | 232 | 13.8 | 282 | 14.64 | 264 | 15.48 | 254 |
| 12.14 | 248 | 12.98 | 264 | 13.82 | 286 | 14.66 | 273 | 15.5 | 233 |
| 12.16 | 231 | 13 | 270 | 13.84 | 300 | 14.68 | 243 | 15.52 | 246 |
| 12.18 | 231 | 13.02 | 228 | 13.86 | 308 | 14.7 | 262 | 15.54 | 245 |
| 12.2 | 247 | 13.04 | 271 | 13.88 | 304 | 14.72 | 292 | 15.56 | 278 |
| 12.22 | 262 | 13.06 | 238 | 13.9 | 278 | 14.74 | 257 | 15.58 | 232 |
| 15.6 | 275 | 16.44 | 755 | 17.28 | 243 | 18.12 | 339 | 18.96 | 907 |
| 15.62 | 287 | 16.46 | 708 | 17.3 | 270 | 18.14 | 302 | 18.98 | 1103 |
| 15.64 | 258 | 16.48 | 568 | 17.32 | 229 | 18.16 | 266 | 19 | 1317 |
| 15.66 | 273 | 16.5 | 443 | 17.34 | 263 | 18.18 | 257 | 19.02 | 1186 |
| 15.68 | 277 | 16.52 | 353 | 17.36 | 232 | 18.2 | 259 | 19.04 | 949 |
| 15.7 | 250 | 16.54 | 267 | 17.38 | 266 | 18.22 | 252 | 19.06 | 753 |
| 15.72 | 225 | 16.56 | 254 | 17.4 | 322 | 18.24 | 248 | 19.08 | 502 |
| 15.74 | 238 | 16.58 | 262 | 17.42 | 354 | 18.26 | 235 | 19.1 | 377 |
| 15.76 | 244 | 16.6 | 275 | 17.44 | 351 | 18.28 | 257 | 19.12 | 269 |
| 15.78 | 235 | 16.62 | 263 | 17.46 | 446 | 18.3 | 233 | 19.14 | 284 |
| 15.8 | 249 | 16.64 | 248 | 17.48 | 466 | 18.32 | 257 | 19.16 | 245 |
| 15.82 | 251 | 16.66 | 273 | 17.5 | 445 | 18.34 | 230 | 19.18 | 257 |
| 15.84 | 249 | 16.68 | 242 | 17.52 | 392 | 18.36 | 247 | 19.2 | 266 |
| 15.86 | 243 | 16.7 | 222 | 17.54 | 380 | 18.38 | 260 | 19.22 | 223 |
| 15.88 | 247 | 16.72 | 280 | 17.56 | 318 | 18.4 | 254 | 19.24 | 250 |
| 15.9 | 267 | 16.74 | 235 | 17.58 | 367 | 18.42 | 244 | 19.26 | 251 |
| 15.92 | 295 | 16.76 | 245 | 17.6 | 424 | 18.44 | 251 | 19.28 | 273 |
| 15.94 | 258 | 16.78 | 260 | 17.62 | 523 | 18.46 | 236 | 19.3 | 246 |
| 15.96 | 274 | 16.8 | 246 | 17.64 | 623 | 18.48 | 253 | 19.32 | 258 |
| 15.98 | 255 | 16.82 | 270 | 17.66 | 675 | 18.5 | 236 | 19.34 | 241 |
| 16 | 239 | 16.84 | 213 | 17.68 | 633 | 18.52 | 260 | 19.36 | 269 |
| 16.02 | 272 | 16.86 | 227 | 17.7 | 700 | 18.54 | 234 | 19.38 | 248 |
| 16.04 | 262 | 16.88 | 251 | 17.72 | 582 | 18.56 | 264 | 19.4 | 242 |
| 16.06 | 266 | 16.9 | 227 | 17.74 | 503 | 18.58 | 255 | 19.42 | 229 |
| 16.08 | 265 | 16.92 | 274 | 17.76 | 431 | 18.6 | 268 | 19.44 | 258 |
| 16.1 | 278 | 16.94 | 246 | 17.78 | 354 | 18.62 | 236 | 19.46 | 241 |
| 16.12 | 243 | 16.96 | 260 | 17.8 | 311 | 18.64 | 227 | 19.48 | 261 |
| 16.14 | 279 | 16.98 | 237 | 17.82 | 281 | 18.66 | 272 | 19.5 | 233 |
| 16.16 | 280 | 17 | 260 | 17.84 | 337 | 18.68 | 254 | 19.52 | 252 |
| 16.18 | 276 | 17.02 | 240 | 17.86 | 348 | 18.7 | 261 | 19.54 | 213 |
| 16.2 | 275 | 17.04 | 267 | 17.88 | 334 | 18.72 | 240 | 19.56 | 243 |
| 16.22 | 297 | 17.06 | 263 | 17.9 | 366 | 18.74 | 287 | 19.58 | 262 |
| 16.24 | 315 | 17.08 | 237 | 17.92 | 452 | 18.76 | 250 | 19.6 | 255 |
| 16.26 | 323 | 17.1 | 261 | 17.94 | 535 | 18.78 | 251 | 19.62 | 237 |
| 16.28 | 333 | 17.12 | 274 | 17.96 | 709 | 18.8 | 275 | 19.64 | 251 |
| 16.3 | 383 | 17.14 | 285 | 17.98 | 901 | 18.82 | 293 | 19.66 | 256 |
| 16.32 | 406 | 17.16 | 270 | 18 | 969 | 18.84 | 336 | 19.68 | 247 |
| 16.34 | 401 | 17.18 | 251 | 18.02 | 1074 | 18.86 | 331 | 19.7 | 214 |
| 16.36 | 428 | 17.2 | 251 | 18.04 | 887 | 18.88 | 373 | 19.72 | 235 |
| 16.38 | 414 | 17.22 | 253 | 18.06 | 764 | 18.9 | 412 | 19.74 | 237 |
| 16.4 | 498 | 17.24 | 264 | 18.08 | 555 | 18.92 | 447 | 19.76 | 242 |
| 16.42 | 609 | 17.26 | 246 | 18.1 | 441 | 18.94 | 582 | 19.78 | 262 |
| 19.8 | 245 | 20.64 | 265 | 21.48 | 272 | 22.32 | 296 | 23.16 | 238 |
| 19.82 | 252 | 20.66 | 259 | 21.5 | 279 | 22.34 | 296 | 23.18 | 254 |
| 19.84 | 254 | 20.68 | 272 | 21.52 | 256 | 22.36 | 297 | 23.2 | 257 |
| 19.86 | 235 | 20.7 | 225 | 21.54 | 241 | 22.38 | 298 | 23.22 | 236 |
| 19.88 | 258 | 20.72 | 257 | 21.56 | 265 | 22.4 | 300 | 23.24 | 247 |
| 19.9 | 237 | 20.74 | 256 | 21.58 | 254 | 22.42 | 329 | 23.26 | 227 |
| 19.92 | 222 | 20.76 | 252 | 21.6 | 213 | 22.44 | 326 | 23.28 | 271 |
| 19.94 | 251 | 20.78 | 257 | 21.62 | 238 | 22.46 | 475 | 23.3 | 241 |
| 19.96 | 263 | 20.8 | 253 | 21.64 | 251 | 22.48 | 728 | 23.32 | 224 |
| 19.98 | 233 | 20.82 | 271 | 21.66 | 269 | 22.5 | 958 | 23.34 | 230 |
| 20 | 264 | 20.84 | 322 | 21.68 | 266 | 22.52 | 978 | 23.36 | 223 |
| 20.02 | 272 | 20.86 | 313 | 21.7 | 238 | 22.54 | 832 | 23.38 | 262 |
| 20.04 | 251 | 20.88 | 320 | 21.72 | 248 | 22.56 | 703 | 23.4 | 277 |
| 20.06 | 253 | 20.9 | 317 | 21.74 | 234 | 22.58 | 605 | 23.42 | 277 |
| 20.08 | 248 | 20.92 | 291 | 21.76 | 216 | 22.6 | 416 | 23.44 | 261 |
| 20.1 | 245 | 20.94 | 278 | 21.78 | 249 | 22.62 | 325 | 23.46 | 266 |
| 20.12 | 287 | 20.96 | 257 | 21.8 | 218 | 22.64 | 317 | 23.48 | 280 |
| 20.14 | 266 | 20.98 | 274 | 21.82 | 258 | 22.66 | 330 | 23.5 | 272 |
| 20.16 | 274 | 21 | 264 | 21.84 | 241 | 22.68 | 307 | 23.52 | 301 |
| 20.18 | 288 | 21.02 | 242 | 21.86 | 262 | 22.7 | 286 | 23.54 | 256 |
| 20.2 | 272 | 21.04 | 238 | 21.88 | 248 | 22.72 | 272 | 23.56 | 339 |
| 20.22 | 232 | 21.06 | 246 | 21.9 | 242 | 22.74 | 286 | 23.58 | 359 |
| 20.24 | 263 | 21.08 | 282 | 21.92 | 228 | 22.76 | 296 | 23.6 | 370 |
| 20.26 | 277 | 21.1 | 264 | 21.94 | 274 | 22.78 | 246 | 23.62 | 385 |
| 20.28 | 242 | 21.12 | 248 | 21.96 | 244 | 22.8 | 296 | 23.64 | 331 |
| 20.3 | 254 | 21.14 | 275 | 21.98 | 241 | 22.82 | 248 | 23.66 | 319 |
| 20.32 | 236 | 21.16 | 271 | 22 | 261 | 22.84 | 246 | 23.68 | 306 |
| 20.34 | 260 | 21.18 | 254 | 22.02 | 261 | 22.86 | 242 | 23.7 | 285 |
| 20.36 | 219 | 21.2 | 272 | 22.04 | 235 | 22.88 | 273 | 23.72 | 244 |
| 20.38 | 241 | 21.22 | 241 | 22.06 | 249 | 22.9 | 263 | 23.74 | 244 |
| 20.4 | 253 | 21.24 | 247 | 22.08 | 247 | 22.92 | 247 | 23.76 | 244 |
| 20.42 | 241 | 21.26 | 228 | 22.1 | 264 | 22.94 | 279 | 23.78 | 237 |
| 20.44 | 249 | 21.28 | 217 | 22.12 | 229 | 22.96 | 225 | 23.8 | 255 |
| 20.46 | 247 | 21.3 | 242 | 22.14 | 243 | 22.98 | 263 | 23.82 | 254 |
| 20.48 | 268 | 21.32 | 241 | 22.16 | 277 | 23 | 221 | 23.84 | 240 |
| 20.5 | 278 | 21.34 | 267 | 22.18 | 279 | 23.02 | 255 | 23.86 | 292 |
| 20.52 | 309 | 21.36 | 276 | 22.2 | 257 | 23.04 | 248 | 23.88 | 236 |
| 20.54 | 274 | 21.38 | 262 | 22.22 | 272 | 23.06 | 280 | 23.9 | 228 |
| 20.56 | 243 | 21.4 | 280 | 22.24 | 288 | 23.08 | 243 | 23.92 | 283 |
| 20.58 | 268 | 21.42 | 260 | 22.26 | 285 | 23.1 | 255 | 23.94 | 245 |
| 20.6 | 281 | 21.44 | 285 | 22.28 | 298 | 23.12 | 257 | 23.96 | 258 |
| 20.62 | 266 | 21.46 | 309 | 22.3 | 319 | 23.14 | 256 | 23.98 | 247 |
| 24 | 254 | 24.84 | 233 | 25.68 | 252 | 26.52 | 258 | 27.36 | 236 |
| 24.02 | 264 | 24.86 | 261 | 25.7 | 230 | 26.54 | 228 | 27.38 | 241 |
| 24.04 | 261 | 24.88 | 223 | 25.72 | 276 | 26.56 | 224 | 27.4 | 209 |
| 24.06 | 236 | 24.9 | 247 | 25.74 | 242 | 26.58 | 234 | 27.42 | 206 |
| 24.08 | 241 | 24.92 | 239 | 25.76 | 254 | 26.6 | 230 | 27.44 | 252 |
| 24.1 | 243 | 24.94 | 197 | 25.78 | 270 | 26.62 | 237 | 27.46 | 268 |
| 24.12 | 257 | 24.96 | 270 | 25.8 | 275 | 26.64 | 233 | 27.48 | 267 |
| 24.14 | 252 | 24.98 | 259 | 25.82 | 279 | 26.66 | 223 | 27.5 | 324 |
| 24.16 | 242 | 25 | 212 | 25.84 | 258 | 26.68 | 210 | 27.52 | 306 |
| 24.18 | 252 | 25.02 | 270 | 25.86 | 260 | 26.7 | 225 | 27.54 | 340 |
| 24.2 | 241 | 25.04 | 261 | 25.88 | 276 | 26.72 | 241 | 27.56 | 319 |
| 24.22 | 226 | 25.06 | 267 | 25.9 | 243 | 26.74 | 208 | 27.58 | 310 |
| 24.24 | 234 | 25.08 | 212 | 25.92 | 229 | 26.76 | 258 | 27.6 | 269 |
| 24.26 | 242 | 25.1 | 250 | 25.94 | 232 | 26.78 | 242 | 27.62 | 258 |
| 24.28 | 240 | 25.12 | 230 | 25.96 | 241 | 26.8 | 274 | 27.64 | 233 |
| 24.3 | 232 | 25.14 | 237 | 25.98 | 216 | 26.82 | 223 | 27.66 | 217 |
| 24.32 | 261 | 25.16 | 243 | 26 | 238 | 26.84 | 251 | 27.68 | 203 |
| 24.34 | 254 | 25.18 | 235 | 26.02 | 261 | 26.86 | 252 | 27.7 | 216 |
| 24.36 | 213 | 25.2 | 229 | 26.04 | 237 | 26.88 | 230 | 27.72 | 229 |
| 24.38 | 236 | 25.22 | 236 | 26.06 | 213 | 26.9 | 240 | 27.74 | 227 |
| 24.4 | 265 | 25.24 | 236 | 26.08 | 240 | 26.92 | 223 | 27.76 | 224 |
| 24.42 | 271 | 25.26 | 227 | 26.1 | 214 | 26.94 | 240 | 27.78 | 207 |
| 24.44 | 251 | 25.28 | 255 | 26.12 | 201 | 26.96 | 256 | 27.8 | 222 |
| 24.46 | 245 | 25.3 | 237 | 26.14 | 224 | 26.98 | 237 | 27.82 | 248 |
| 24.48 | 279 | 25.32 | 226 | 26.16 | 227 | 27 | 224 | 27.84 | 250 |
| 24.5 | 274 | 25.34 | 226 | 26.18 | 222 | 27.02 | 234 | 27.86 | 331 |
| 24.52 | 294 | 25.36 | 256 | 26.2 | 210 | 27.04 | 242 | 27.88 | 438 |
| 24.54 | 307 | 25.38 | 253 | 26.22 | 211 | 27.06 | 252 | 27.9 | 565 |
| 24.56 | 318 | 25.4 | 258 | 26.24 | 232 | 27.08 | 245 | 27.92 | 663 |
| 24.58 | 295 | 25.42 | 256 | 26.26 | 231 | 27.1 | 261 | 27.94 | 620 |
| 24.6 | 257 | 25.44 | 232 | 26.28 | 214 | 27.12 | 256 | 27.96 | 533 |
| 24.62 | 267 | 25.46 | 278 | 26.3 | 235 | 27.14 | 232 | 27.98 | 506 |
| 24.64 | 251 | 25.48 | 256 | 26.32 | 231 | 27.16 | 217 | 28 | 436 |
| 24.66 | 252 | 25.5 | 233 | 26.34 | 252 | 27.18 | 227 | 28.02 | 337 |
| 24.68 | 276 | 25.52 | 250 | 26.36 | 229 | 27.2 | 211 | 28.04 | 295 |
| 24.7 | 245 | 25.54 | 253 | 26.38 | 210 | 27.22 | 196 | 28.06 | 257 |
| 24.72 | 235 | 25.56 | 237 | 26.4 | 234 | 27.24 | 196 | 28.08 | 229 |
| 24.74 | 260 | 25.58 | 223 | 26.42 | 243 | 27.26 | 226 | 28.1 | 246 |
| 24.76 | 232 | 25.6 | 250 | 26.44 | 218 | 27.28 | 253 | 28.12 | 220 |
| 24.78 | 217 | 25.62 | 227 | 26.46 | 215 | 27.3 | 245 | 28.14 | 213 |
| 24.8 | 250 | 25.64 | 249 | 26.48 | 239 | 27.32 | 207 | 28.16 | 220 |
| 24.82 | 249 | 25.66 | 242 | 26.5 | 227 | 27.34 | 231 | 28.18 | 214 |
| 28.2 | 211 | 29.04 | 212 | 29.88 | 194 | 30.72 | 225 | 31.56 | 185 |
| 28.22 | 211 | 29.06 | 250 | 29.9 | 243 | 30.74 | 185 | 31.58 | 177 |
| 28.24 | 229 | 29.08 | 236 | 29.92 | 211 | 30.76 | 167 | 31.6 | 172 |
| 28.26 | 201 | 29.1 | 235 | 29.94 | 221 | 30.78 | 178 | 31.62 | 198 |
| 28.28 | 238 | 29.12 | 245 | 29.96 | 184 | 30.8 | 200 | 31.64 | 221 |
| 28.3 | 211 | 29.14 | 237 | 29.98 | 189 | 30.82 | 201 | 31.66 | 199 |
| 28.32 | 203 | 29.16 | 222 | 30 | 219 | 30.84 | 206 | 31.68 | 177 |
| 28.34 | 199 | 29.18 | 231 | 30.02 | 202 | 30.86 | 202 | 31.7 | 193 |
| 28.36 | 227 | 29.2 | 211 | 30.04 | 192 | 30.88 | 174 | 31.72 | 184 |
| 28.38 | 203 | 29.22 | 245 | 30.06 | 179 | 30.9 | 190 | 31.74 | 187 |
| 28.4 | 210 | 29.24 | 232 | 30.08 | 202 | 30.92 | 201 | 31.76 | 187 |
| 28.42 | 193 | 29.26 | 249 | 30.1 | 189 | 30.94 | 197 | 31.78 | 176 |
| 28.44 | 217 | 29.28 | 272 | 30.12 | 206 | 30.96 | 178 | 31.8 | 189 |
| 28.46 | 251 | 29.3 | 301 | 30.14 | 211 | 30.98 | 197 | 31.82 | 187 |
| 28.48 | 223 | 29.32 | 302 | 30.16 | 223 | 31 | 165 | 31.84 | 197 |
| 28.5 | 220 | 29.34 | 294 | 30.18 | 218 | 31.02 | 202 | 31.86 | 166 |
| 28.52 | 237 | 29.36 | 273 | 30.2 | 187 | 31.04 | 182 | 31.88 | 163 |
| 28.54 | 241 | 29.38 | 262 | 30.22 | 183 | 31.06 | 193 | 31.9 | 171 |
| 28.56 | 233 | 29.4 | 224 | 30.24 | 214 | 31.08 | 170 | 31.92 | 159 |
| 28.58 | 242 | 29.42 | 259 | 30.26 | 205 | 31.1 | 181 | 31.94 | 152 |
| 28.6 | 224 | 29.44 | 205 | 30.28 | 206 | 31.12 | 193 | 31.96 | 180 |
| 28.62 | 242 | 29.46 | 197 | 30.3 | 218 | 31.14 | 223 | 31.98 | 185 |
| 28.64 | 261 | 29.48 | 227 | 30.32 | 205 | 31.16 | 207 | 32 | 184 |
| 28.66 | 257 | 29.5 | 228 | 30.34 | 182 | 31.18 | 183 | 32.02 | 209 |
| 28.68 | 243 | 29.52 | 195 | 30.36 | 208 | 31.2 | 160 | 32.04 | 169 |
| 28.7 | 253 | 29.54 | 184 | 30.38 | 211 | 31.22 | 173 | 32.06 | 162 |
| 28.72 | 237 | 29.56 | 211 | 30.4 | 205 | 31.24 | 198 | 32.08 | 175 |
| 28.74 | 248 | 29.58 | 205 | 30.42 | 204 | 31.26 | 181 | 32.1 | 182 |
| 28.76 | 199 | 29.6 | 191 | 30.44 | 153 | 31.28 | 203 | 32.12 | 155 |
| 28.78 | 224 | 29.62 | 223 | 30.46 | 174 | 31.3 | 180 | 32.14 | 174 |
| 28.8 | 237 | 29.64 | 175 | 30.48 | 191 | 31.32 | 179 | 32.16 | 166 |
| 28.82 | 213 | 29.66 | 197 | 30.5 | 161 | 31.34 | 181 | 32.18 | 199 |
| 28.84 | 247 | 29.68 | 223 | 30.52 | 189 | 31.36 | 186 | 32.2 | 156 |
| 28.86 | 198 | 29.7 | 209 | 30.54 | 188 | 31.38 | 201 | 32.22 | 182 |
| 28.88 | 196 | 29.72 | 197 | 30.56 | 192 | 31.4 | 201 | 32.24 | 154 |
| 28.9 | 207 | 29.74 | 172 | 30.58 | 213 | 31.42 | 201 | 32.26 | 174 |
| 28.92 | 204 | 29.76 | 183 | 30.6 | 202 | 31.44 | 173 | 32.28 | 183 |
| 28.94 | 222 | 29.78 | 223 | 30.62 | 187 | 31.46 | 152 | 32.3 | 192 |
| 28.96 | 215 | 29.8 | 196 | 30.64 | 185 | 31.48 | 163 | 32.32 | 188 |
| 28.98 | 186 | 29.82 | 181 | 30.66 | 190 | 31.5 | 180 | 32.34 | 183 |
| 29 | 231 | 29.84 | 190 | 30.68 | 203 | 31.52 | 151 | 32.36 | 217 |
| 29.02 | 216 | 29.86 | 206 | 30.7 | 197 | 31.54 | 170 | 32.38 | 204 |
| 32.4 | 166 | 33.24 | 189 | 34.08 | 234 | 34.92 | 173 | | |
| 32.42 | 215 | 33.26 | 183 | 34.1 | 223 | 34.94 | 157 | | |
| 32.44 | 211 | 33.28 | 161 | 34.12 | 217 | 34.96 | 153 | | |
| 32.46 | 196 | 33.3 | 156 | 34.14 | 196 | 34.98 | 146 | | |
| 32.48 | 181 | 33.32 | 181 | 34.16 | 200 | 35 | 138 | | |
| 32.5 | 178 | 33.34 | 173 | 34.18 | 197 | | | | |
| 32.52 | 198 | 33.36 | 160 | 34.2 | 195 | | | | |
| 32.54 | 159 | 33.38 | 202 | 34.22 | 172 | | | | |
| 32.56 | 161 | 33.4 | 164 | 34.24 | 161 | | | | |
| 32.58 | 176 | 33.42 | 143 | 34.26 | 179 | | | | |
| 32.6 | 192 | 33.44 | 183 | 34.28 | 150 | | | | |
| 32.62 | 176 | 33.46 | 178 | 34.3 | 144 | | | | |
| 32.64 | 162 | 33.48 | 168 | 34.32 | 163 | | | | |
| 32.66 | 172 | 33.5 | 173 | 34.34 | 146 | | | | |
| 32.68 | 174 | 33.52 | 139 | 34.36 | 135 | | | | |
| 32.7 | 200 | 33.54 | 148 | 34.38 | 166 | | | | |
| 32.72 | 180 | 33.56 | 161 | 34.4 | 129 | | | | |
| 32.74 | 171 | 33.58 | 166 | 34.42 | 167 | | | | |
| 32.76 | 159 | 33.6 | 159 | 34.44 | 136 | | | | |
| 32.78 | 162 | 33.62 | 150 | 34.46 | 140 | | | | |
| 32.8 | 181 | 33.64 | 135 | 34.48 | 134 | | | | |
| 32.82 | 169 | 33.66 | 142 | 34.5 | 149 | | | | |
| 32.84 | 169 | 33.68 | 170 | 34.52 | 148 | | | | |
| 32.86 | 185 | 33.7 | 158 | 34.54 | 169 | | | | |
| 32.88 | 186 | 33.72 | 144 | 34.56 | 147 | | | | |
| 32.9 | 186 | 33.74 | 155 | 34.58 | 153 | | | | |
| 32.92 | 166 | 33.76 | 171 | 34.6 | 151 | | | | |
| 32.94 | 162 | 33.78 | 159 | 34.62 | 140 | | | | |
| 32.96 | 166 | 33.8 | 177 | 34.64 | 149 | | | | |
| 32.98 | 156 | 33.82 | 167 | 34.66 | 171 | | | | |
| 33 | 170 | 33.84 | 167 | 34.68 | 147 | | | | |
| 33.02 | 193 | 33.86 | 157 | 34.7 | 152 | | | | |
| 33.04 | 180 | 33.88 | 160 | 34.72 | 150 | | | | |
| 33.06 | 180 | 33.9 | 160 | 34.74 | 170 | | | | |
| 33.08 | 190 | 33.92 | 174 | 34.76 | 150 | | | | |
| 33.1 | 161 | 33.94 | 161 | 34.78 | 160 | | | | |
| 33.12 | 180 | 33.96 | 160 | 34.8 | 142 | | | | |
| 33.14 | 166 | 33.98 | 187 | 34.82 | 162 | | | | |
| 33.16 | 170 | 34 | 173 | 34.84 | 148 | | | | |
| 33.18 | 154 | 34.02 | 183 | 34.86 | 170 | | | | |
| 33.2 | 175 | 34.04 | 246 | 34.88 | 146 | | | | |
| 33.22 | 187 | 34.06 | 223 | 34.9 | 162 | | | | |

FIG. 20

| 2Theta | 108 | 2Theta | 108 | 2Theta | 108 | 2Theta | 108 | 2Theta | 108 |
|---|---|---|---|---|---|---|---|---|---|
| 3 | 112 | 3.84 | 95 | 4.68 | 89 | 5.52 | 94 | 6.36 | 77 |
| 3.02 | 110 | 3.86 | 94 | 4.7 | 86 | 5.54 | 90 | 6.38 | 83 |
| 3.04 | 101 | 3.88 | 95 | 4.72 | 94 | 5.56 | 87 | 6.4 | 87 |
| 3.06 | 127 | 3.9 | 96 | 4.74 | 86 | 5.58 | 86 | 6.42 | 85 |
| 3.08 | 101 | 3.92 | 92 | 4.76 | 98 | 5.6 | 90 | 6.44 | 81 |
| 3.1 | 129 | 3.94 | 84 | 4.78 | 86 | 5.62 | 101 | 6.46 | 81 |
| 3.12 | 91 | 3.96 | 91 | 4.8 | 99 | 5.64 | 86 | 6.48 | 100 |
| 3.14 | 113 | 3.98 | 92 | 4.82 | 86 | 5.66 | 85 | 6.5 | 67 |
| 3.16 | 101 | 4 | 113 | 4.84 | 91 | 5.68 | 83 | 6.52 | 56 |
| 3.18 | 117 | 4.02 | 87 | 4.86 | 103 | 5.7 | 96 | 6.54 | 105 |
| 3.2 | 96 | 4.04 | 106 | 4.88 | 79 | 5.72 | 99 | 6.56 | 72 |
| 3.22 | 107 | 4.06 | 92 | 4.9 | 82 | 5.74 | 77 | 6.58 | 91 |
| 3.24 | 107 | 4.08 | 102 | 4.92 | 94 | 5.76 | 97 | 6.6 | 72 |
| 3.26 | 109 | 4.1 | 110 | 4.94 | 96 | 5.78 | 87 | 6.62 | 82 |
| 3.28 | 98 | 4.12 | 110 | 4.96 | 88 | 5.8 | 87 | 6.64 | 75 |
| 3.3 | 120 | 4.14 | 99 | 4.98 | 92 | 5.82 | 100 | 6.66 | 85 |
| 3.32 | 93 | 4.16 | 103 | 5 | 105 | 5.84 | 108 | 6.68 | 81 |
| 3.34 | 99 | 4.18 | 92 | 5.02 | 88 | 5.86 | 88 | 6.7 | 89 |
| 3.36 | 95 | 4.2 | 101 | 5.04 | 98 | 5.88 | 94 | 6.72 | 71 |
| 3.38 | 109 | 4.22 | 85 | 5.06 | 95 | 5.9 | 85 | 6.74 | 78 |
| 3.4 | 117 | 4.24 | 92 | 5.08 | 83 | 5.92 | 89 | 6.76 | 80 |
| 3.42 | 88 | 4.26 | 79 | 5.1 | 78 | 5.94 | 96 | 6.78 | 86 |
| 3.44 | 109 | 4.28 | 104 | 5.12 | 84 | 5.96 | 85 | 6.8 | 88 |
| 3.46 | 97 | 4.3 | 91 | 5.14 | 82 | 5.98 | 98 | 6.82 | 80 |
| 3.48 | 110 | 4.32 | 98 | 5.16 | 103 | 6 | 72 | 6.84 | 76 |
| 3.5 | 90 | 4.34 | 91 | 5.18 | 81 | 6.02 | 83 | 6.86 | 60 |
| 3.52 | 97 | 4.36 | 92 | 5.2 | 81 | 6.04 | 86 | 6.88 | 83 |
| 3.54 | 104 | 4.38 | 118 | 5.22 | 76 | 6.06 | 89 | 6.9 | 79 |
| 3.56 | 105 | 4.4 | 83 | 5.24 | 111 | 6.08 | 102 | 6.92 | 76 |
| 3.58 | 86 | 4.42 | 88 | 5.26 | 95 | 6.1 | 89 | 6.94 | 92 |
| 3.6 | 87 | 4.44 | 73 | 5.28 | 89 | 6.12 | 82 | 6.96 | 68 |
| 3.62 | 112 | 4.46 | 97 | 5.3 | 87 | 6.14 | 79 | 6.98 | 82 |
| 3.64 | 108 | 4.48 | 94 | 5.32 | 95 | 6.16 | 67 | 7 | 78 |
| 3.66 | 108 | 4.5 | 86 | 5.34 | 97 | 6.18 | 70 | 7.02 | 76 |
| 3.68 | 110 | 4.52 | 86 | 5.36 | 104 | 6.2 | 79 | 7.04 | 74 |
| 3.7 | 108 | 4.54 | 97 | 5.38 | 85 | 6.22 | 85 | 7.06 | 76 |
| 3.72 | 87 | 4.56 | 105 | 5.4 | 87 | 6.24 | 92 | 7.08 | 78 |
| 3.74 | 105 | 4.58 | 80 | 5.42 | 96 | 6.26 | 77 | 7.1 | 80 |
| 3.76 | 97 | 4.6 | 85 | 5.44 | 101 | 6.28 | 74 | 7.12 | 97 |
| 3.78 | 112 | 4.62 | 87 | 5.46 | 81 | 6.3 | 78 | 7.14 | 65 |
| 3.8 | 89 | 4.64 | 109 | 5.48 | 85 | 6.32 | 77 | 7.16 | 70 |
| 3.82 | 90 | 4.66 | 83 | 5.5 | 88 | 6.34 | 85 | 7.18 | 80 |
| 7.2 | 82 | 8.04 | 74 | 8.88 | 130 | 9.72 | 114 | 10.56 | 139 |
| 7.22 | 75 | 8.06 | 90 | 8.9 | 101 | 9.74 | 119 | 10.58 | 121 |
| 7.24 | 72 | 8.08 | 87 | 8.92 | 136 | 9.76 | 121 | 10.6 | 131 |
| 7.26 | 82 | 8.1 | 87 | 8.94 | 114 | 9.78 | 96 | 10.62 | 121 |
| 7.28 | 82 | 8.12 | 78 | 8.96 | 122 | 9.8 | 127 | 10.64 | 117 |
| 7.3 | 69 | 8.14 | 89 | 8.98 | 111 | 9.82 | 106 | 10.66 | 132 |
| 7.32 | 76 | 8.16 | 80 | 9 | 120 | 9.84 | 126 | 10.68 | 124 |
| 7.34 | 72 | 8.18 | 94 | 9.02 | 131 | 9.86 | 88 | 10.7 | 131 |
| 7.36 | 75 | 8.2 | 79 | 9.04 | 116 | 9.88 | 97 | 10.72 | 133 |
| 7.38 | 80 | 8.22 | 72 | 9.06 | 117 | 9.9 | 114 | 10.74 | 144 |
| 7.4 | 72 | 8.24 | 80 | 9.08 | 111 | 9.92 | 112 | 10.76 | 131 |
| 7.42 | 73 | 8.26 | 81 | 9.1 | 102 | 9.94 | 124 | 10.78 | 135 |
| 7.44 | 98 | 8.28 | 75 | 9.12 | 113 | 9.96 | 139 | 10.8 | 132 |
| 7.46 | 85 | 8.3 | 102 | 9.14 | 105 | 9.98 | 108 | 10.82 | 129 |
| 7.48 | 72 | 8.32 | 93 | 9.16 | 113 | 10 | 134 | 10.84 | 138 |
| 7.5 | 79 | 8.34 | 88 | 9.18 | 88 | 10.02 | 127 | 10.86 | 141 |
| 7.52 | 90 | 8.36 | 90 | 9.2 | 103 | 10.04 | 119 | 10.88 | 125 |
| 7.54 | 84 | 8.38 | 85 | 9.22 | 121 | 10.06 | 114 | 10.9 | 137 |
| 7.56 | 65 | 8.4 | 79 | 9.24 | 107 | 10.08 | 109 | 10.92 | 117 |
| 7.58 | 92 | 8.42 | 89 | 9.26 | 99 | 10.1 | 117 | 10.94 | 143 |
| 7.6 | 69 | 8.44 | 93 | 9.28 | 142 | 10.12 | 128 | 10.96 | 161 |
| 7.62 | 86 | 8.46 | 100 | 9.3 | 114 | 10.14 | 131 | 10.98 | 150 |
| 7.64 | 88 | 8.48 | 88 | 9.32 | 105 | 10.16 | 127 | 11 | 136 |
| 7.66 | 91 | 8.5 | 90 | 9.34 | 106 | 10.18 | 135 | 11.02 | 164 |
| 7.68 | 71 | 8.52 | 91 | 9.36 | 127 | 10.2 | 123 | 11.04 | 145 |
| 7.7 | 70 | 8.54 | 80 | 9.38 | 120 | 10.22 | 118 | 11.06 | 143 |
| 7.72 | 78 | 8.56 | 89 | 9.4 | 113 | 10.24 | 121 | 11.08 | 138 |
| 7.74 | 80 | 8.58 | 93 | 9.42 | 103 | 10.26 | 139 | 11.1 | 143 |
| 7.76 | 83 | 8.6 | 78 | 9.44 | 105 | 10.28 | 107 | 11.12 | 156 |
| 7.78 | 73 | 8.62 | 92 | 9.46 | 118 | 10.3 | 119 | 11.14 | 162 |
| 7.8 | 94 | 8.64 | 99 | 9.48 | 122 | 10.32 | 138 | 11.16 | 127 |
| 7.82 | 59 | 8.66 | 82 | 9.5 | 115 | 10.34 | 140 | 11.18 | 144 |
| 7.84 | 92 | 8.68 | 100 | 9.52 | 108 | 10.36 | 128 | 11.2 | 150 |
| 7.86 | 99 | 8.7 | 107 | 9.54 | 122 | 10.38 | 126 | 11.22 | 151 |
| 7.88 | 81 | 8.72 | 96 | 9.56 | 96 | 10.4 | 123 | 11.24 | 135 |
| 7.9 | 82 | 8.74 | 93 | 9.58 | 97 | 10.42 | 144 | 11.26 | 150 |
| 7.92 | 84 | 8.76 | 91 | 9.6 | 115 | 10.44 | 136 | 11.28 | 144 |
| 7.94 | 88 | 8.78 | 86 | 9.62 | 106 | 10.46 | 131 | 11.3 | 129 |
| 7.96 | 100 | 8.8 | 114 | 9.64 | 117 | 10.48 | 126 | 11.32 | 171 |
| 7.98 | 101 | 8.82 | 106 | 9.66 | 118 | 10.5 | 100 | 11.34 | 143 |
| 8 | 74 | 8.84 | 106 | 9.68 | 115 | 10.52 | 139 | 11.36 | 150 |
| 8.02 | 84 | 8.86 | 103 | 9.7 | 97 | 10.54 | 135 | 11.38 | 161 |
| 11.4 | 161 | 12.24 | 167 | 13.08 | 161 | 13.92 | 260 | 14.76 | 262 |
| 11.42 | 156 | 12.26 | 165 | 13.1 | 182 | 13.94 | 221 | 14.78 | 266 |
| 11.44 | 156 | 12.28 | 183 | 13.12 | 189 | 13.96 | 257 | 14.8 | 217 |
| 11.46 | 154 | 12.3 | 155 | 13.14 | 206 | 13.98 | 256 | 14.82 | 210 |
| 11.48 | 152 | 12.32 | 186 | 13.16 | 197 | 14 | 262 | 14.84 | 228 |
| 11.5 | 153 | 12.34 | 180 | 13.18 | 181 | 14.02 | 246 | 14.86 | 209 |
| 11.52 | 180 | 12.36 | 176 | 13.2 | 199 | 14.04 | 222 | 14.88 | 215 |
| 11.54 | 174 | 12.38 | 179 | 13.22 | 200 | 14.06 | 246 | 14.9 | 212 |
| 11.56 | 144 | 12.4 | 170 | 13.24 | 234 | 14.08 | 230 | 14.92 | 210 |
| 11.58 | 162 | 12.42 | 172 | 13.26 | 179 | 14.1 | 195 | 14.94 | 186 |
| 11.6 | 177 | 12.44 | 164 | 13.28 | 224 | 14.12 | 235 | 14.96 | 193 |
| 11.62 | 156 | 12.46 | 170 | 13.3 | 180 | 14.14 | 192 | 14.98 | 198 |
| 11.64 | 155 | 12.48 | 185 | 13.32 | 169 | 14.16 | 191 | 15 | 198 |
| 11.66 | 156 | 12.5 | 182 | 13.34 | 194 | 14.18 | 224 | 15.02 | 197 |
| 11.68 | 146 | 12.52 | 164 | 13.36 | 195 | 14.2 | 197 | 15.04 | 184 |
| 11.7 | 154 | 12.54 | 177 | 13.38 | 196 | 14.22 | 191 | 15.06 | 220 |
| 11.72 | 151 | 12.56 | 195 | 13.4 | 180 | 14.24 | 213 | 15.08 | 197 |
| 11.74 | 134 | 12.58 | 166 | 13.42 | 182 | 14.26 | 201 | 15.1 | 210 |
| 11.76 | 154 | 12.6 | 186 | 13.44 | 187 | 14.28 | 190 | 15.12 | 189 |
| 11.78 | 162 | 12.62 | 168 | 13.46 | 178 | 14.3 | 231 | 15.14 | 195 |
| 11.8 | 170 | 12.64 | 186 | 13.48 | 183 | 14.32 | 221 | 15.16 | 176 |
| 11.82 | 143 | 12.66 | 186 | 13.5 | 191 | 14.34 | 216 | 15.18 | 224 |
| 11.84 | 174 | 12.68 | 168 | 13.52 | 212 | 14.36 | 210 | 15.2 | 183 |
| 11.86 | 164 | 12.7 | 167 | 13.54 | 164 | 14.38 | 219 | 15.22 | 184 |
| 11.88 | 183 | 12.72 | 179 | 13.56 | 197 | 14.4 | 224 | 15.24 | 192 |
| 11.9 | 132 | 12.74 | 175 | 13.58 | 219 | 14.42 | 227 | 15.26 | 195 |
| 11.92 | 170 | 12.76 | 171 | 13.6 | 172 | 14.44 | 226 | 15.28 | 161 |
| 11.94 | 148 | 12.78 | 172 | 13.62 | 192 | 14.46 | 201 | 15.3 | 192 |
| 11.96 | 173 | 12.8 | 172 | 13.64 | 204 | 14.48 | 206 | 15.32 | 205 |
| 11.98 | 135 | 12.82 | 185 | 13.66 | 203 | 14.5 | 255 | 15.34 | 177 |
| 12 | 158 | 12.84 | 210 | 13.68 | 188 | 14.52 | 232 | 15.36 | 186 |
| 12.02 | 164 | 12.86 | 181 | 13.7 | 196 | 14.54 | 237 | 15.38 | 201 |
| 12.04 | 158 | 12.88 | 177 | 13.72 | 194 | 14.56 | 263 | 15.4 | 211 |
| 12.06 | 169 | 12.9 | 177 | 13.74 | 208 | 14.58 | 286 | 15.42 | 198 |
| 12.08 | 172 | 12.92 | 155 | 13.76 | 190 | 14.6 | 311 | 15.44 | 199 |
| 12.1 | 166 | 12.94 | 226 | 13.78 | 207 | 14.62 | 277 | 15.46 | 188 |
| 12.12 | 171 | 12.96 | 191 | 13.8 | 211 | 14.64 | 297 | 15.48 | 197 |
| 12.14 | 149 | 12.98 | 183 | 13.82 | 247 | 14.66 | 310 | 15.5 | 188 |
| 12.16 | 178 | 13 | 197 | 13.84 | 231 | 14.68 | 264 | 15.52 | 207 |
| 12.18 | 130 | 13.02 | 189 | 13.86 | 237 | 14.7 | 277 | 15.54 | 184 |
| 12.2 | 179 | 13.04 | 192 | 13.88 | 235 | 14.72 | 266 | 15.56 | 190 |
| 12.22 | 158 | 13.06 | 170 | 13.9 | 264 | 14.74 | 256 | 15.58 | 166 |
| 15.6 | 176 | 16.44 | 169 | 17.28 | 207 | 18.12 | 561 | 18.96 | 185 |
| 15.62 | 243 | 16.46 | 187 | 17.3 | 184 | 18.14 | 458 | 18.98 | 240 |
| 15.64 | 214 | 16.48 | 207 | 17.32 | 193 | 18.16 | 387 | 19 | 195 |
| 15.66 | 237 | 16.5 | 192 | 17.34 | 194 | 18.18 | 329 | 19.02 | 179 |
| 15.68 | 217 | 16.52 | 167 | 17.36 | 187 | 18.2 | 289 | 19.04 | 181 |
| 15.7 | 215 | 16.54 | 199 | 17.38 | 204 | 18.22 | 262 | 19.06 | 196 |
| 15.72 | 220 | 16.56 | 200 | 17.4 | 183 | 18.24 | 259 | 19.08 | 234 |
| 15.74 | 229 | 16.58 | 175 | 17.42 | 206 | 18.26 | 212 | 19.1 | 234 |
| 15.76 | 231 | 16.6 | 172 | 17.44 | 176 | 18.28 | 234 | 19.12 | 231 |
| 15.78 | 265 | 16.62 | 173 | 17.46 | 181 | 18.3 | 210 | 19.14 | 273 |
| 15.8 | 260 | 16.64 | 184 | 17.48 | 191 | 18.32 | 198 | 19.16 | 295 |
| 15.82 | 262 | 16.66 | 186 | 17.5 | 208 | 18.34 | 219 | 19.18 | 282 |
| 15.84 | 261 | 16.68 | 179 | 17.52 | 164 | 18.36 | 183 | 19.2 | 312 |
| 15.86 | 380 | 16.7 | 207 | 17.54 | 213 | 18.38 | 180 | 19.22 | 353 |
| 15.88 | 327 | 16.72 | 180 | 17.56 | 204 | 18.4 | 187 | 19.24 | 305 |
| 15.9 | 357 | 16.74 | 201 | 17.58 | 193 | 18.42 | 213 | 19.26 | 307 |
| 15.92 | 338 | 16.76 | 193 | 17.6 | 185 | 18.44 | 196 | 19.28 | 290 |
| 15.94 | 349 | 16.78 | 186 | 17.62 | 199 | 18.46 | 191 | 19.3 | 302 |
| 15.96 | 334 | 16.8 | 167 | 17.64 | 200 | 18.48 | 192 | 19.32 | 261 |
| 15.98 | 311 | 16.82 | 176 | 17.66 | 219 | 18.5 | 181 | 19.34 | 242 |
| 16 | 291 | 16.84 | 178 | 17.68 | 217 | 18.52 | 194 | 19.36 | 238 |
| 16.02 | 293 | 16.86 | 184 | 17.7 | 241 | 18.54 | 197 | 19.38 | 211 |
| 16.04 | 253 | 16.88 | 194 | 17.72 | 238 | 18.56 | 210 | 19.4 | 233 |
| 16.06 | 217 | 16.9 | 160 | 17.74 | 247 | 18.58 | 192 | 19.42 | 216 |
| 16.08 | 238 | 16.92 | 165 | 17.76 | 237 | 18.6 | 186 | 19.44 | 203 |
| 16.1 | 225 | 16.94 | 212 | 17.78 | 276 | 18.62 | 210 | 19.46 | 201 |
| 16.12 | 200 | 16.96 | 190 | 17.8 | 293 | 18.64 | 206 | 19.48 | 185 |
| 16.14 | 216 | 16.98 | 182 | 17.82 | 292 | 18.66 | 212 | 19.5 | 213 |
| 16.16 | 189 | 17 | 181 | 17.84 | 323 | 18.68 | 175 | 19.52 | 186 |
| 16.18 | 204 | 17.02 | 171 | 17.86 | 340 | 18.7 | 180 | 19.54 | 204 |
| 16.2 | 199 | 17.04 | 182 | 17.88 | 409 | 18.72 | 181 | 19.56 | 209 |
| 16.22 | 214 | 17.06 | 198 | 17.9 | 459 | 18.74 | 194 | 19.58 | 215 |
| 16.24 | 211 | 17.08 | 182 | 17.92 | 505 | 18.76 | 203 | 19.6 | 229 |
| 16.26 | 209 | 17.1 | 171 | 17.94 | 606 | 18.78 | 196 | 19.62 | 233 |
| 16.28 | 156 | 17.12 | 167 | 17.96 | 653 | 18.8 | 196 | 19.64 | 259 |
| 16.3 | 181 | 17.14 | 172 | 17.98 | 737 | 18.82 | 182 | 19.66 | 220 |
| 16.32 | 186 | 17.16 | 164 | 18 | 730 | 18.84 | 197 | 19.68 | 215 |
| 16.34 | 200 | 17.18 | 165 | 18.02 | 788 | 18.86 | 188 | 19.7 | 226 |
| 16.36 | 208 | 17.2 | 209 | 18.04 | 758 | 18.88 | 181 | 19.72 | 215 |
| 16.38 | 200 | 17.22 | 172 | 18.06 | 765 | 18.9 | 160 | 19.74 | 222 |
| 16.4 | 191 | 17.24 | 165 | 18.08 | 703 | 18.92 | 198 | 19.76 | 200 |
| 16.42 | 192 | 17.26 | 185 | 18.1 | 630 | 18.94 | 204 | 19.78 | 195 |
| 19.8 | 181 | 20.64 | 209 | 21.48 | 172 | 22.32 | 189 | 23.16 | 174 |
| 19.82 | 216 | 20.66 | 176 | 21.5 | 177 | 22.34 | 200 | 23.18 | 202 |
| 19.84 | 192 | 20.68 | 176 | 21.52 | 221 | 22.36 | 214 | 23.2 | 163 |
| 19.86 | 158 | 20.7 | 198 | 21.54 | 163 | 22.38 | 190 | 23.22 | 199 |
| 19.88 | 182 | 20.72 | 174 | 21.56 | 197 | 22.4 | 236 | 23.24 | 192 |
| 19.9 | 170 | 20.74 | 217 | 21.58 | 177 | 22.42 | 204 | 23.26 | 170 |
| 19.92 | 208 | 20.76 | 182 | 21.6 | 180 | 22.44 | 226 | 23.28 | 172 |
| 19.94 | 203 | 20.78 | 204 | 21.62 | 179 | 22.46 | 214 | 23.3 | 191 |
| 19.96 | 182 | 20.8 | 225 | 21.64 | 179 | 22.48 | 210 | 23.32 | 179 |
| 19.98 | 171 | 20.82 | 229 | 21.66 | 204 | 22.5 | 184 | 23.34 | 178 |
| 20 | 198 | 20.84 | 253 | 21.68 | 190 | 22.52 | 188 | 23.36 | 178 |
| 20.02 | 187 | 20.86 | 251 | 21.7 | 168 | 22.54 | 223 | 23.38 | 181 |
| 20.04 | 195 | 20.88 | 244 | 21.72 | 171 | 22.56 | 200 | 23.4 | 204 |
| 20.06 | 185 | 20.9 | 227 | 21.74 | 168 | 22.58 | 212 | 23.42 | 189 |
| 20.08 | 172 | 20.92 | 260 | 21.76 | 174 | 22.6 | 211 | 23.44 | 191 |
| 20.1 | 187 | 20.94 | 237 | 21.78 | 144 | 22.62 | 220 | 23.46 | 188 |
| 20.12 | 165 | 20.96 | 261 | 21.8 | 184 | 22.64 | 237 | 23.48 | 212 |
| 20.14 | 174 | 20.98 | 213 | 21.82 | 178 | 22.66 | 242 | 23.5 | 193 |
| 20.16 | 186 | 21 | 201 | 21.84 | 192 | 22.68 | 257 | 23.52 | 206 |
| 20.18 | 195 | 21.02 | 221 | 21.86 | 162 | 22.7 | 240 | 23.54 | 212 |
| 20.2 | 171 | 21.04 | 184 | 21.88 | 182 | 22.72 | 240 | 23.56 | 227 |
| 20.22 | 164 | 21.06 | 196 | 21.9 | 177 | 22.74 | 281 | 23.58 | 218 |
| 20.24 | 173 | 21.08 | 174 | 21.92 | 170 | 22.76 | 325 | 23.6 | 231 |
| 20.26 | 201 | 21.1 | 185 | 21.94 | 172 | 22.78 | 315 | 23.62 | 248 |
| 20.28 | 164 | 21.12 | 203 | 21.96 | 165 | 22.8 | 294 | 23.64 | 226 |
| 20.3 | 177 | 21.14 | 185 | 21.98 | 174 | 22.82 | 309 | 23.66 | 296 |
| 20.32 | 161 | 21.16 | 187 | 22 | 172 | 22.84 | 335 | 23.68 | 284 |
| 20.34 | 174 | 21.18 | 178 | 22.02 | 195 | 22.86 | 294 | 23.7 | 299 |
| 20.36 | 199 | 21.2 | 173 | 22.04 | 160 | 22.88 | 322 | 23.72 | 331 |
| 20.38 | 156 | 21.22 | 181 | 22.06 | 196 | 22.9 | 287 | 23.74 | 348 |
| 20.4 | 203 | 21.24 | 192 | 22.08 | 199 | 22.92 | 293 | 23.76 | 374 |
| 20.42 | 177 | 21.26 | 199 | 22.1 | 201 | 22.94 | 246 | 23.78 | 357 |
| 20.44 | 182 | 21.28 | 160 | 22.12 | 165 | 22.96 | 245 | 23.8 | 376 |
| 20.46 | 177 | 21.3 | 187 | 22.14 | 187 | 22.98 | 218 | 23.82 | 342 |
| 20.48 | 167 | 21.32 | 172 | 22.16 | 193 | 23 | 232 | 23.84 | 329 |
| 20.5 | 176 | 21.34 | 164 | 22.18 | 202 | 23.02 | 213 | 23.86 | 297 |
| 20.52 | 168 | 21.36 | 200 | 22.2 | 188 | 23.04 | 185 | 23.88 | 306 |
| 20.54 | 183 | 21.38 | 193 | 22.22 | 211 | 23.06 | 202 | 23.9 | 272 |
| 20.56 | 192 | 21.4 | 186 | 22.24 | 193 | 23.08 | 208 | 23.92 | 249 |
| 20.58 | 207 | 21.42 | 178 | 22.26 | 195 | 23.1 | 203 | 23.94 | 231 |
| 20.6 | 184 | 21.44 | 178 | 22.28 | 187 | 23.12 | 180 | 23.96 | 216 |
| 20.62 | 213 | 21.46 | 188 | 22.3 | 211 | 23.14 | 195 | 23.98 | 197 |
| 24 | 201 | 24.84 | 473 | 25.68 | 190 | 26.52 | 163 | 27.36 | 176 |
| 24.02 | 206 | 24.86 | 492 | 25.7 | 167 | 26.54 | 181 | 27.38 | 165 |
| 24.04 | 205 | 24.88 | 473 | 25.72 | 175 | 26.56 | 167 | 27.4 | 183 |
| 24.06 | 214 | 24.9 | 453 | 25.74 | 179 | 26.58 | 157 | 27.42 | 171 |
| 24.08 | 199 | 24.92 | 478 | 25.76 | 181 | 26.6 | 185 | 27.44 | 184 |
| 24.1 | 202 | 24.94 | 436 | 25.78 | 161 | 26.62 | 193 | 27.46 | 179 |
| 24.12 | 200 | 24.96 | 382 | 25.8 | 182 | 26.64 | 173 | 27.48 | 153 |
| 24.14 | 181 | 24.98 | 357 | 25.82 | 170 | 26.66 | 189 | 27.5 | 157 |
| 24.16 | 177 | 25 | 332 | 25.84 | 182 | 26.68 | 219 | 27.52 | 170 |
| 24.18 | 160 | 25.02 | 277 | 25.86 | 171 | 26.7 | 176 | 27.54 | 178 |
| 24.2 | 199 | 25.04 | 236 | 25.88 | 155 | 26.72 | 175 | 27.56 | 160 |
| 24.22 | 178 | 25.06 | 213 | 25.9 | 158 | 26.74 | 205 | 27.58 | 185 |
| 24.24 | 194 | 25.08 | 234 | 25.92 | 162 | 26.76 | 179 | 27.6 | 156 |
| 24.26 | 173 | 25.1 | 208 | 25.94 | 156 | 26.78 | 177 | 27.62 | 171 |
| 24.28 | 178 | 25.12 | 184 | 25.96 | 172 | 26.8 | 189 | 27.64 | 152 |
| 24.3 | 194 | 25.14 | 180 | 25.98 | 172 | 26.82 | 191 | 27.66 | 172 |
| 24.32 | 180 | 25.16 | 193 | 26 | 173 | 26.84 | 172 | 27.68 | 169 |
| 24.34 | 186 | 25.18 | 197 | 26.02 | 165 | 26.86 | 166 | 27.7 | 157 |
| 24.36 | 174 | 25.2 | 202 | 26.04 | 155 | 26.88 | 166 | 27.72 | 151 |
| 24.38 | 185 | 25.22 | 173 | 26.06 | 148 | 26.9 | 183 | 27.74 | 175 |
| 24.4 | 176 | 25.24 | 204 | 26.08 | 155 | 26.92 | 160 | 27.76 | 150 |
| 24.42 | 193 | 25.26 | 178 | 26.1 | 157 | 26.94 | 179 | 27.78 | 166 |
| 24.44 | 204 | 25.28 | 163 | 26.12 | 154 | 26.96 | 165 | 27.8 | 192 |
| 24.46 | 192 | 25.3 | 197 | 26.14 | 154 | 26.98 | 157 | 27.82 | 159 |
| 24.48 | 199 | 25.32 | 188 | 26.16 | 193 | 27 | 166 | 27.84 | 191 |
| 24.5 | 203 | 25.34 | 187 | 26.18 | 182 | 27.02 | 182 | 27.86 | 172 |
| 24.52 | 188 | 25.36 | 175 | 26.2 | 182 | 27.04 | 164 | 27.88 | 171 |
| 24.54 | 213 | 25.38 | 168 | 26.22 | 184 | 27.06 | 198 | 27.9 | 183 |
| 24.56 | 206 | 25.4 | 167 | 26.24 | 155 | 27.08 | 153 | 27.92 | 181 |
| 24.58 | 240 | 25.42 | 188 | 26.26 | 176 | 27.1 | 182 | 27.94 | 213 |
| 24.6 | 222 | 25.44 | 182 | 26.28 | 176 | 27.12 | 196 | 27.96 | 213 |
| 24.62 | 236 | 25.46 | 192 | 26.3 | 177 | 27.14 | 198 | 27.98 | 183 |
| 24.64 | 257 | 25.48 | 178 | 26.32 | 170 | 27.16 | 179 | 28 | 216 |
| 24.66 | 261 | 25.5 | 174 | 26.34 | 173 | 27.18 | 162 | 28.02 | 227 |
| 24.68 | 234 | 25.52 | 180 | 26.36 | 175 | 27.2 | 173 | 28.04 | 223 |
| 24.7 | 297 | 25.54 | 173 | 26.38 | 174 | 27.22 | 180 | 28.06 | 249 |
| 24.72 | 313 | 25.56 | 194 | 26.4 | 162 | 27.24 | 216 | 28.08 | 236 |
| 24.74 | 368 | 25.58 | 195 | 26.42 | 171 | 27.26 | 191 | 28.1 | 256 |
| 24.76 | 418 | 25.6 | 158 | 26.44 | 185 | 27.28 | 206 | 28.12 | 245 |
| 24.78 | 398 | 25.62 | 153 | 26.46 | 173 | 27.3 | 220 | 28.14 | 279 |
| 24.8 | 457 | 25.64 | 157 | 26.48 | 178 | 27.32 | 175 | 28.16 | 286 |
| 24.82 | 472 | 25.66 | 160 | 26.5 | 180 | 27.34 | 196 | 28.18 | 313 |
| 28.2 | 327 | 29.04 | 342 | 29.88 | 166 | 30.72 | 140 | 31.56 | 135 |
| 28.22 | 356 | 29.06 | 363 | 29.9 | 165 | 30.74 | 136 | 31.58 | 148 |
| 28.24 | 354 | 29.08 | 338 | 29.92 | 203 | 30.76 | 154 | 31.6 | 120 |
| 28.26 | 314 | 29.1 | 313 | 29.94 | 159 | 30.78 | 136 | 31.62 | 153 |
| 28.28 | 309 | 29.12 | 318 | 29.96 | 152 | 30.8 | 121 | 31.64 | 137 |
| 28.3 | 319 | 29.14 | 289 | 29.98 | 181 | 30.82 | 138 | 31.66 | 132 |
| 28.32 | 269 | 29.16 | 256 | 30 | 163 | 30.84 | 129 | 31.68 | 142 |
| 28.34 | 305 | 29.18 | 290 | 30.02 | 164 | 30.86 | 148 | 31.7 | 141 |
| 28.36 | 246 | 29.2 | 250 | 30.04 | 166 | 30.88 | 132 | 31.72 | 137 |
| 28.38 | 254 | 29.22 | 232 | 30.06 | 153 | 30.9 | 138 | 31.74 | 123 |
| 28.4 | 239 | 29.24 | 190 | 30.08 | 149 | 30.92 | 164 | 31.76 | 127 |
| 28.42 | 240 | 29.26 | 218 | 30.1 | 164 | 30.94 | 121 | 31.78 | 136 |
| 28.44 | 214 | 29.28 | 198 | 30.12 | 138 | 30.96 | 152 | 31.8 | 121 |
| 28.46 | 210 | 29.3 | 201 | 30.14 | 132 | 30.98 | 146 | 31.82 | 141 |
| 28.48 | 173 | 29.32 | 206 | 30.16 | 145 | 31 | 118 | 31.84 | 138 |
| 28.5 | 186 | 29.34 | 198 | 30.18 | 157 | 31.02 | 126 | 31.86 | 129 |
| 28.52 | 179 | 29.36 | 151 | 30.2 | 137 | 31.04 | 131 | 31.88 | 127 |
| 28.54 | 170 | 29.38 | 161 | 30.22 | 161 | 31.06 | 145 | 31.9 | 148 |
| 28.56 | 166 | 29.4 | 171 | 30.24 | 132 | 31.08 | 142 | 31.92 | 148 |
| 28.58 | 165 | 29.42 | 170 | 30.26 | 130 | 31.1 | 138 | 31.94 | 123 |
| 28.6 | 147 | 29.44 | 172 | 30.28 | 141 | 31.12 | 161 | 31.96 | 135 |
| 28.62 | 182 | 29.46 | 149 | 30.3 | 149 | 31.14 | 138 | 31.98 | 139 |
| 28.64 | 173 | 29.48 | 159 | 30.32 | 147 | 31.16 | 144 | 32 | 148 |
| 28.66 | 174 | 29.5 | 161 | 30.34 | 146 | 31.18 | 132 | 32.02 | 140 |
| 28.68 | 154 | 29.52 | 157 | 30.36 | 129 | 31.2 | 164 | 32.04 | 136 |
| 28.7 | 163 | 29.54 | 136 | 30.38 | 164 | 31.22 | 143 | 32.06 | 138 |
| 28.72 | 186 | 29.56 | 149 | 30.4 | 196 | 31.24 | 129 | 32.08 | 127 |
| 28.74 | 157 | 29.58 | 165 | 30.42 | 149 | 31.26 | 115 | 32.1 | 140 |
| 28.76 | 132 | 29.6 | 147 | 30.44 | 154 | 31.28 | 128 | 32.12 | 117 |
| 28.78 | 196 | 29.62 | 143 | 30.46 | 141 | 31.3 | 136 | 32.14 | 119 |
| 28.8 | 185 | 29.64 | 158 | 30.48 | 163 | 31.32 | 153 | 32.16 | 136 |
| 28.82 | 171 | 29.66 | 170 | 30.5 | 150 | 31.34 | 137 | 32.18 | 150 |
| 28.84 | 195 | 29.68 | 150 | 30.52 | 150 | 31.36 | 139 | 32.2 | 154 |
| 28.86 | 201 | 29.7 | 140 | 30.54 | 135 | 31.38 | 143 | 32.22 | 141 |
| 28.88 | 201 | 29.72 | 175 | 30.56 | 143 | 31.4 | 131 | 32.24 | 136 |
| 28.9 | 232 | 29.74 | 144 | 30.58 | 166 | 31.42 | 133 | 32.26 | 105 |
| 28.92 | 260 | 29.76 | 157 | 30.6 | 164 | 31.44 | 144 | 32.28 | 142 |
| 28.94 | 266 | 29.78 | 166 | 30.62 | 164 | 31.46 | 117 | 32.3 | 143 |
| 28.96 | 279 | 29.8 | 184 | 30.64 | 143 | 31.48 | 136 | 32.32 | 146 |
| 28.98 | 325 | 29.82 | 139 | 30.66 | 157 | 31.5 | 127 | 32.34 | 133 |
| 29 | 325 | 29.84 | 167 | 30.68 | 150 | 31.52 | 143 | 32.36 | 114 |
| 29.02 | 331 | 29.86 | 182 | 30.7 | 143 | 31.54 | 114 | 32.38 | 143 |
| 32.4 | 156 | 33.24 | 173 | 34.08 | 127 | 34.92 | 120 | | |
| 32.42 | 123 | 33.26 | 179 | 34.1 | 142 | 34.94 | 120 | | |
| 32.44 | 149 | 33.28 | 179 | 34.12 | 111 | 34.96 | 104 | | |
| 32.46 | 136 | 33.3 | 169 | 34.14 | 130 | 34.98 | 102 | | |
| 32.48 | 152 | 33.32 | 170 | 34.16 | 154 | 35 | 114 | | |
| 32.5 | 160 | 33.34 | 168 | 34.18 | 119 | | | | |
| 32.52 | 165 | 33.36 | 157 | 34.2 | 125 | | | | |
| 32.54 | 125 | 33.38 | 165 | 34.22 | 133 | | | | |
| 32.56 | 132 | 33.4 | 150 | 34.24 | 125 | | | | |
| 32.58 | 152 | 33.42 | 146 | 34.26 | 122 | | | | |
| 32.6 | 155 | 33.44 | 152 | 34.28 | 140 | | | | |
| 32.62 | 126 | 33.46 | 133 | 34.3 | 108 | | | | |
| 32.64 | 140 | 33.48 | 129 | 34.32 | 93 | | | | |
| 32.66 | 130 | 33.5 | 141 | 34.34 | 97 | | | | |
| 32.68 | 151 | 33.52 | 122 | 34.36 | 111 | | | | |
| 32.7 | 144 | 33.54 | 124 | 34.38 | 129 | | | | |
| 32.72 | 172 | 33.56 | 139 | 34.4 | 141 | | | | |
| 32.74 | 142 | 33.58 | 117 | 34.42 | 103 | | | | |
| 32.76 | 157 | 33.6 | 125 | 34.44 | 127 | | | | |
| 32.78 | 162 | 33.62 | 117 | 34.46 | 115 | | | | |
| 32.8 | 165 | 33.64 | 109 | 34.48 | 134 | | | | |
| 32.82 | 147 | 33.66 | 134 | 34.5 | 109 | | | | |
| 32.84 | 134 | 33.68 | 136 | 34.52 | 113 | | | | |
| 32.86 | 157 | 33.7 | 102 | 34.54 | 101 | | | | |
| 32.88 | 139 | 33.72 | 131 | 34.56 | 120 | | | | |
| 32.9 | 140 | 33.74 | 135 | 34.58 | 120 | | | | |
| 32.92 | 142 | 33.76 | 131 | 34.6 | 104 | | | | |
| 32.94 | 140 | 33.78 | 125 | 34.62 | 134 | | | | |
| 32.96 | 143 | 33.8 | 118 | 34.64 | 121 | | | | |
| 32.98 | 129 | 33.82 | 130 | 34.66 | 115 | | | | |
| 33 | 139 | 33.84 | 111 | 34.68 | 117 | | | | |
| 33.02 | 130 | 33.86 | 132 | 34.7 | 133 | | | | |
| 33.04 | 161 | 33.88 | 114 | 34.72 | 116 | | | | |
| 33.06 | 142 | 33.9 | 112 | 34.74 | 109 | | | | |
| 33.08 | 151 | 33.92 | 127 | 34.76 | 114 | | | | |
| 33.1 | 162 | 33.94 | 124 | 34.78 | 130 | | | | |
| 33.12 | 163 | 33.96 | 122 | 34.8 | 138 | | | | |
| 33.14 | 125 | 33.98 | 119 | 34.82 | 120 | | | | |
| 33.16 | 160 | 34 | 109 | 34.84 | 114 | | | | |
| 33.18 | 166 | 34.02 | 117 | 34.86 | 121 | | | | |
| 33.2 | 175 | 34.04 | 134 | 34.88 | 133 | | | | |
| 33.22 | 197 | 34.06 | 123 | 34.9 | 124 | | | | |

### 2.3 Melting Point

The melting point of the compounds were determined and the melting points of the various compounds are listed at Table 3.

TABLE 3
Melting points of the compounds of the present invention

| Compound number | Melting point |
|---|---|
| 101 | 169-175° C. |
| 102 | 170-176° C. |
| 103 | 162-170° C. |
| 104 | 165-178° C. |
| 105 | 158-178° C. |
| 106 | 172-182° C. |

### 2.4 Infrared Spectroscopy (IR) Results

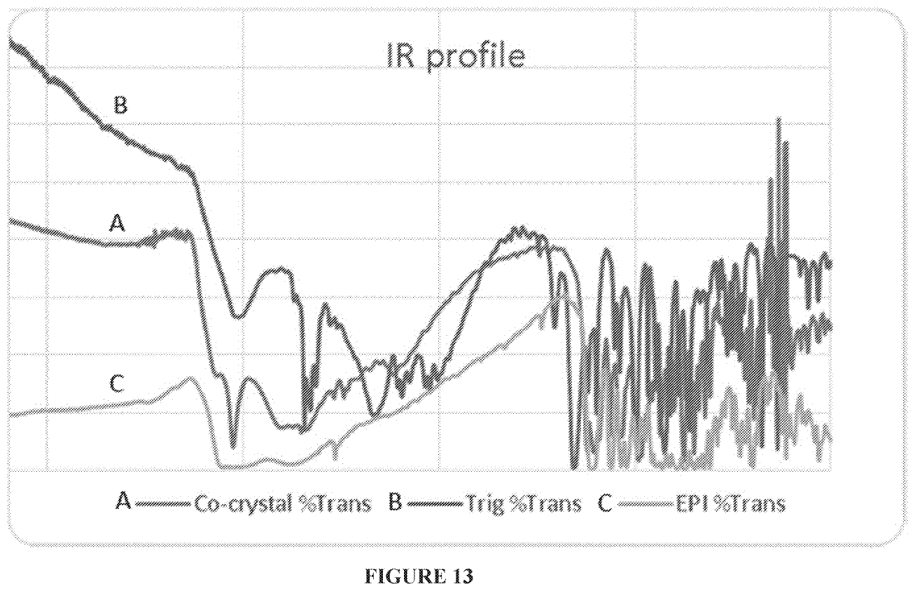

IR profile of epicatechin co-crystal was different from trigonelline and epicatechin parent as shown in FIG. 13.

### Example 3. Pharmacokinetics (PK) Study of the Co-Crystals of the Present Invention in Male SD Rats

Pharmacokinetics study was carried out to evaluate the plasma exposure of (+) epicatechin (Compound 108) and (−) epicatechin (Compound 107) and their corresponding trigonelline (1:1) co-crystals. Co-crystals of the present invention were administered orally (PO) at a dose of 10 mpk equivalent of parent drugs in male SD rats. The PK results of the compound were compared with exposure of parent drugs. The dosing vehicle that was used in this study was CMC and Tween 80. After oral dosing, blood was collected by serial bleeding at different time points in heparinised tubes. Blood samples were centrifuged at 10,000 rpm for 5 min. at 4° C. to obtain the plasma, which were aspirated into separate labelled tubes and stored at −80° C. Extraction solvent was added to plasma, was vortexed and shaken on shaker for 10 minutes, centrifuged at 10,000 rpm for 10 minutes at 4° C. Supernatant was kept for analysis. Acetonitrile and plasma calibration curves were generated and percentage of drug recovery from plasma determined. Quantitative analysis was done by liquid chromatography tandem mass spectrometer (API3000 LC-MS/MS). Cmax, Tmax, AUC and t½ were calculated using Graph Pad PRISM version 5.04 and the results are depicted in Table 4.

TABLE 4
Pharmacokinetic paramaters of the co-
crystals of the present invention

| Parameters | 101 | 104 | 107 | 108 |
|---|---|---|---|---|
| Cmax (nM) | 434.9 | 454.9 | 386.3 | 333.0 |
| Tmax (h) | 1.1 | 0.80 | 0.30 | 1.0 |
| AUC (nM · h) | 756.6 | 745.6 | 678.0 | 579.1 |
| T½ elimination (h) | 0.45 | 0.68 | 1.89 | 0.83 |

### Example 4. Pharmacokinetics (PK) Study of the Co-Crystals of the Present Invention in Male SD Rats

Pharmacokinetics study was carried out to evaluate the plasma exposure of (+) epicatechin (referred to herein as SPR590 or as Compound 108) and its corresponding trigonelline (1:1) co-crystals (referred to herein as SPR515 or as Compound 104). Co-crystals of the present invention were administered orally (PO) at a dose of 10 mpk in male SD rats for the control (+) epicatechin group and 10 mpk of SPR 515 which is equivalent to 6.25 mpk of epicatechin. The PK results of the compound were compared with exposure of parent drugs. The dosing vehicle that was used in this study was CMC and Tween 80. After oral dosing, blood was collected by serial bleeding at different time points in heparinised tubes. Blood samples were centrifuged at 10,000 rpm for 5 min. at 4° C. to obtain the plasma, which were aspirated into separate labelled tubes and stored at −80° C. Extraction solvent was added to plasma, was vortexed and shaken on shaker for 10 minutes, centrifuged at 10,000 rpm for 10 minutes at 4° C. Supernatant was kept for analysis. Acetonitrile and plasma calibration curves were generated and percentage of drug recovery from plasma determined. Quantitative analysis was done by liquid chromatography tandem mass spectrometer (API3000 LC-MS/MS). PK parameters were calculated using Graph Pad PRISM version 5.04 and the results are depicted in Table 5.

TABLE 5
Plasma levels of the co-crystals of the present invention

| Time | | Compound No. 108 ((+) |
|---|---|---|
| points (h) | Compound No. 104 | epicatechin) (nM) |
| 0.16 | 432.4 | 235.6 |
| 0.5 | 620.5 | 337.6 |
| 1 | 764.0 | 261.8 |
| 2 | 418.9 | 151.7 |
| 4 | 95.3 | 24.1 |
| 8 | 0.0 | 0.0 |

TABLE 6
Cmax and AUC of the co-crystals of the present invention

| | | | MW Adjusted | | |
|---|---|---|---|---|---|
| | Dose | | (+)-epicatechin | Cmax | AUC |
| Compound | (mpk) | MW | Dose (mpk) | (nM) | (nM · h) |
| SPR590 | 10 | 290.3 | 10 | 386.0 | 678.0 |
| (Compound 108) | | | | | |
| SPR515 | 10 | 463.8 | 6.25 | 800.7 | 1821.2 |
| (Compound 104) | | | | | |

From, Table 5 and Table 6, above, it can be clearly seen that the co-crystal of the present invention has better pharmacokinetic properties than the (+) epicatechin.

### Example 6. Preparation of (−) Epicatechin_Trigonelline (1:1) Co-Crystals (Compound 101)

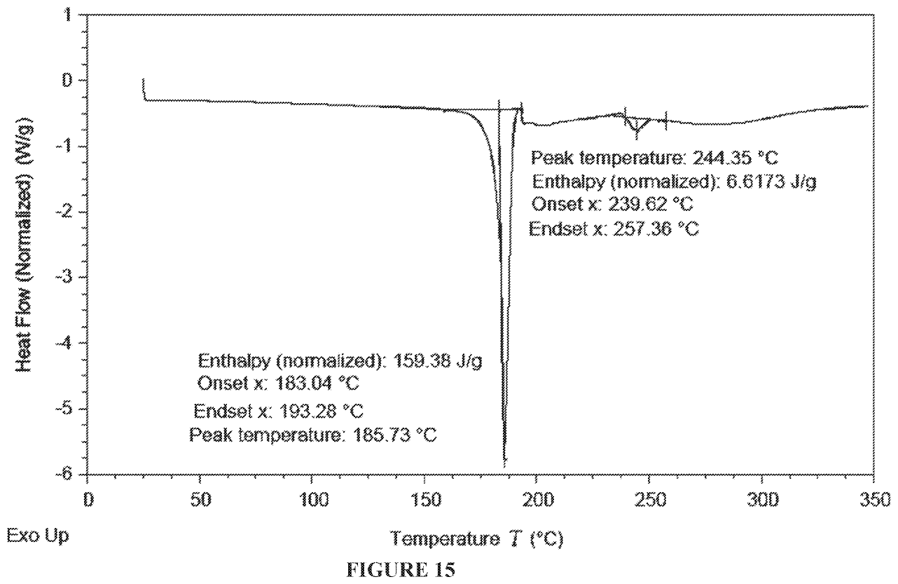

(−) Epicatechin (Compound 107, 1.0 eq.) was taken in isopropanol (20 vol) and warmed at 700° C. To this stirred suspension, trigonelline hydrochloride (1.1 eq) in water (2 vol) was added dropwise. The resulting clear solution was stirred for another 15 min, then cooled to room temperature and the solution kept at 18° C. for 24 h for crystallization. The resulting co-crystal was filtered, washed with cold 10% water in isopropanol (5 vol) and dried. The resulting co-crystal was again taken in ethanol:water (1:1, 5 vol) and stirred for 15 min. The mixture was filtered, washed with ethanol (2 vol) and dried to yield pure co-crystal (Compound 101). The DSC pattern is shown in FIG. 15.

### Example 7. Preparation of (+) Epicatechin_Trigonelline (1:1) Co-Crystals (Compound 104)

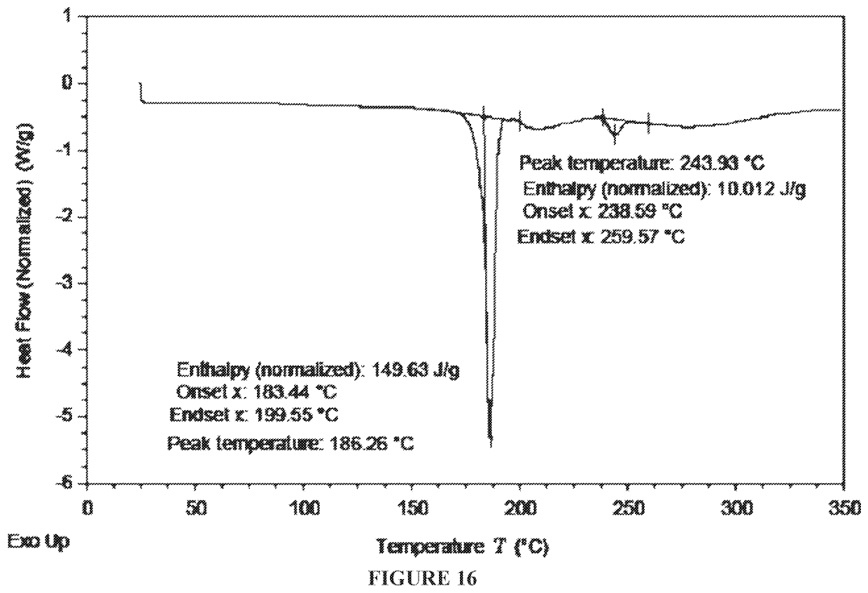

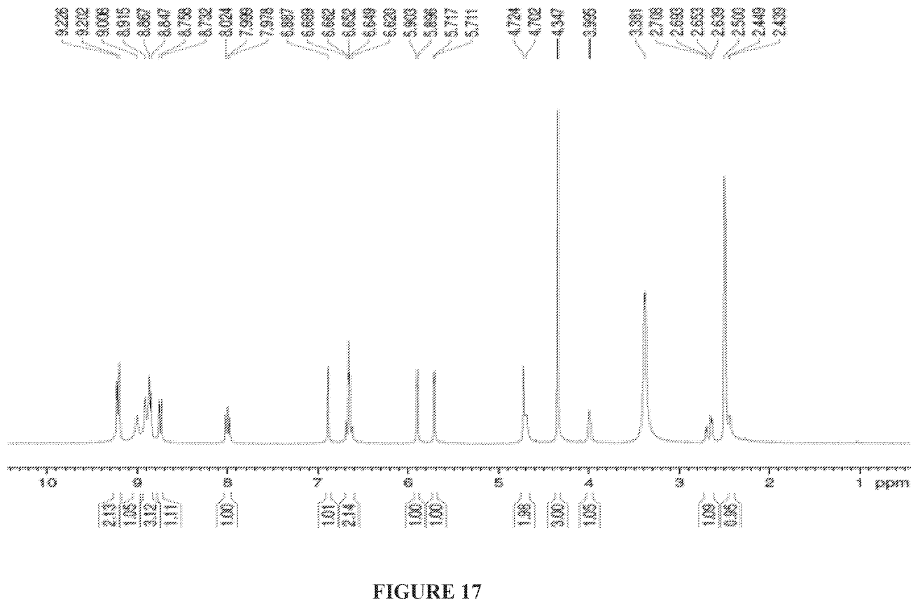

(+) Epicatechin (Compound 108, 1.0 eq.) was taken in isopropanol (20 vol) and warmed at 700° C. To this stirred suspension, trigonelline hydrochloride (1.1 eq) in water (2 vol) was added dropwise. The resulting clear solution was stirred for another 15 min. then cooled to room temperature and the solution kept at 18° C. for 24 h for crystallization. The resulting co-crystal was filtered, washed with cold 10% water in isopropanol (5 vol) and dried. The resulting co-crystal was again taken in ethanol:water (1:1, 5 vol) and stirred for 15 min. The mixture was filtered, washed with Ethanol (2 vol) and dried to yield pure co-crystal (Compound 104). The DSC pattern is shown in FIG. 16, and the ¹H NMR spectrum in DMSO is shown in FIG. 17.

### Example 8. Preparation of (+) Epicatechin_(D)-Proline (1:1) Co-Crystals (Compound 109)

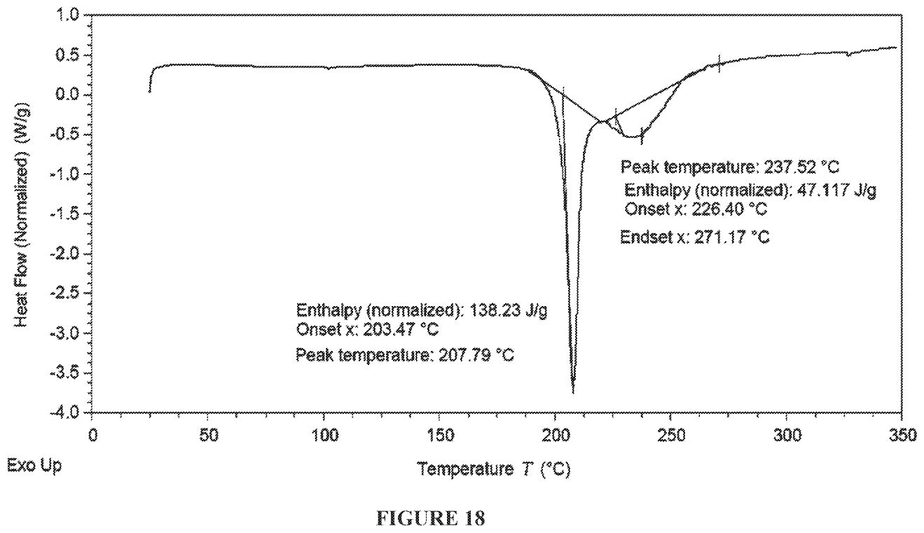

100 mg (+) epicatechin (Compound 108, 1.0 eq.) was taken in isopropanol (7 ml) and heated up at 70° C. for 15 min until a clear solution was obtained. This was followed by addition of 40 mg D-proline and stirring at 70° C. for 15 min. The turbid solution was brought to room temperature and kept for 2 h and then kept at 4° C. (refrigerator) for 16 h. A solid formed, which was filtered and then washed with pentane (5 ml) to yield solid Compound 109. Yield: 49%. The melting point was determined to be in the range of 198-202° C. The DSC pattern is shown in FIG. 18.

### Example 9. Preparation of (−) Epicatechin_(L)-Proline (1:1) Co-Crystals (Compound 110)

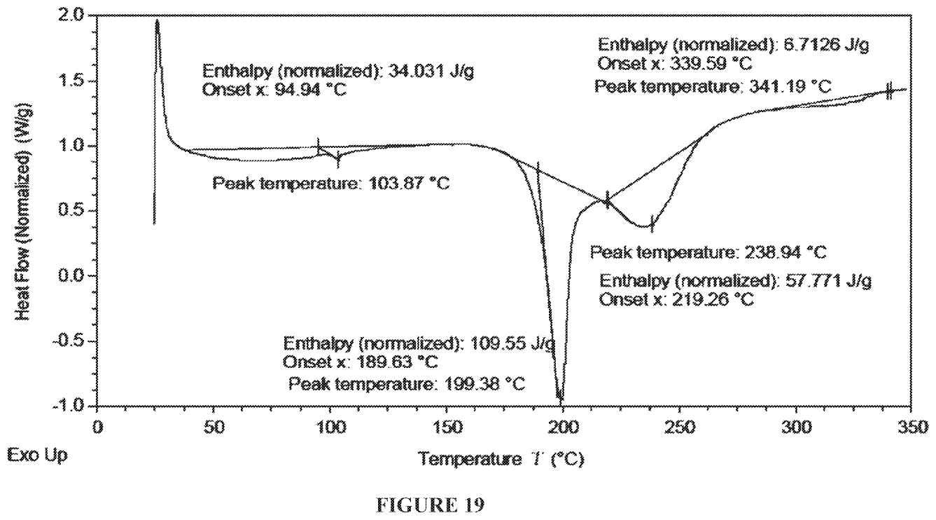

100 mg (−) epicatechin (Compound 107, 1.0 eq.) was taken in isopropanol (7 ml) and heated up at 70° C. for 15 min until a clear solution was obtained. This was followed by addition of 40 mg L-proline and stirring at 70° C. for 15 min. The turbid solution was brought to room temperature and kept for 2 h and then kept at 4° C. (refrigerator) for 16 h. A solid formed, which was filtered and then washed with pentane (5 ml) to yield solid compound Compound 110. Yield: 46%. The melting point was determined to be in the range of 195-198° C. The DSC pattern is shown in FIG. 19.

### Example 10. Pharmacokinetics Study of Compound 109 in Male SD Rats

A pharmacokinetics study was carried out to evaluate the plasma exposure of D-proline co-crystals of (+) epicatechin (Compound 109). Co-crystals were administered orally (PO) at a dose of 14 mpk of Compound 109, which is equivalent to 10 mpk of epicatechin. The dosing vehicle that was used in this study was 0.5% CMC. After oral dosing, blood was collected by serial bleeding at different time points in heparinised tubes. Blood samples were centrifuged at 10,000 rpm for 5 min. at 4° C. to obtain the plasma, which were aspirated into separate labelled tubes and stored at −80° C. Extraction solvent was added to plasma, was vortexed and shaken on a shaker for 10 minutes, and then centrifuged at 10,000 rpm for 10 minutes at 4° C. Supernatant was kept for analysis. Acetonitrile and plasma calibration curves were generated and percentage of drug recovery from plasma determined. Quantitative analysis was done by liquid chromatography tandem mass spectrometer (API3000 LC-MS/MS). PK parameters were calculated using Graph Pad PRISM version 5.04 and the results are depicted in Table 7.

TABLE 7

| Parameter | Average |
|---|---|
| Cmax (nM) | 777.56 |
| Tmax (h) | 0.75 |
| AUC (nM · h) | 1905.33 |
| T½ elimination (h) | 1.71 |

All documents, including patents, patent application and publications cited herein, including all documents cited therein, tables, and drawings, are hereby expressly incorporated by reference in their entirety for all purposes.

While the foregoing written description of the compounds, uses, and methods described herein enables one of ordinary skill in the art to make and use the compounds, uses, and methods described herein, those of ordinary skill in the art will understand and appreciate the existence of variations, combinations, and equivalents of the specific embodiment, method, and examples herein. The compounds, uses, and methods provided herein should therefore not be limited by the above-described embodiments, methods, or examples, but rather encompasses all embodiments and methods within the scope and spirit of the compounds, uses, and methods provided herein.

1. A co-crystal comprising epicatechin and a co-crystal former of Formula (I): 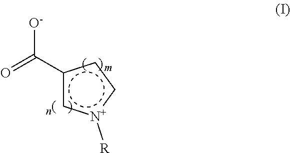
or a stereoisomer thereof,
wherein:
n is 0, 1, 2, or 3;
m is 0, 1, 2, 3, or 4; [symbol]
indicates that ring A is saturated, partially unsaturated, or fully unsaturated; and
R is hydrogen or C₁-C₆ alkyl, wherein the C₁-C₆ alkyl is unsubstituted or substituted with one or more substituents independently selected from the group consisting of halo, —CN, —OH, and C₁-C₆ haloalkyl.

2. The co-crystal of claim 1, wherein the epicatechin is (+) epicatechin.

3. The co-crystal of claim 1, wherein the epicatechin is (−) epicatechin.

4. The co-crystal of any one of claims 1-3, wherein the epicatechin and the co-crystal former of Formula (I) are present in a molar ratio ranging from about 1:3 to about 3:1.

5. The co-crystal of claim 4, wherein the molar ratio is about 1:3, about 1:2, about 1:1, about 2:1 or about 3:1.

6. The co-crystal of any one of claims 1-5, wherein n is 1, m is 2, and ring A is fully unsaturated.

7. The co-crystal of any one of claims 1-5, wherein n is 0, m is 2, and ring A is saturated.

8. The co-crystal of any one of claims 1-7, wherein R is hydrogen or methyl.

9. The co-crystal of any one of claims 1-3, wherein the co-crystal former of Formula (I) is trigonelline.

10. The co-crystal of claim 1, wherein the epicatechin is (−) epicatechin and the co-crystal-former of Formula (I) is trigonelline, wherein the molar ratio of (−) epicatechin:trigonelline is 1:1.

11. The co-crystal of claim 10, wherein the co-crystal exhibits an X-ray diffraction pattern substantially as shown in FIG. 10.

12. The co-crystal of claim 10, wherein the co-crystal is characterized by an X-ray diffraction pattern comprising one or more peaks with 20 values selected from the group consisting of about 17.6°, about 18.0°, about 19.0°, about 13.6°, at about 27.0°, about 16.4°, about 20.9°, about 22.5°, about 23.6°, about 25.0°, about 25.7°, and about 29.0°

13. The co-crystal of claim 10, wherein the co-crystal is characterized by a melting point in a range of 169-175° C.

14. The co-crystal of claim 10, wherein the co-crystal is characterized by an infrared pattern substantially as shown in FIG. 13.

15. The co-crystal of claim 1, wherein the epicatechin is (+) epicatechin and the co-crystal-former of Formula (I) is trigonelline, wherein the molar ratio of (+) epicatechin:trigonelline is 1:1.

16. The co-crystal of claim 15, wherein the co-crystal exhibits an X-ray diffraction pattern substantially as shown in FIG. 11.

17. The co-crystal of claim 15, wherein the co-crystal is characterized by an X-ray diffraction pattern comprising one or more peaks with 2θ values selected from the group consisting of about 13.6°, about 19.0°, about 6.9°, about 16.4°, about 17.6°, about 18.0°, about 22.5°, and about 27.9.

18. The co-crystal of claim 15, wherein the co-crystal is characterized by a melting point in a range of 165-178° C. ° C.

19. The co-crystal of claim 8, wherein the epicatechin is (+) epicatechin and the co-crystal-former of Formula (I) is trigonelline, wherein the molar ratio of (+) epicatechin:trigonelline is 1:2.

20. The co-crystal of claim 19, wherein the co-crystal exhibits an X-ray diffraction pattern substantially as shown in FIG. 12.

21. The co-crystal of claim 19, wherein the co-crystal is characterized by an X-ray diffraction pattern comprising one or more peaks with 2θ values selected from the group consisting of about 13.6, about 19.0°, about 18.0°, about 11.2°, about 16.4°, about 17.7°, about 22.5°, and about 27.9°.

22. The co-crystal of claim 19, wherein the co-crystal is characterized by a melting point in a range of 172-182° C.

23. The co-crystal of any one of claims 1-3, wherein the co-crystal former of Formula (I) is s proline or a stereoisomer thereof.

24. The co-crystal of claim 1, wherein the epicatechin is (+) epicatechin and the co-crystal-former of Formula (I) is D-proline.

25. The co-crystal of claim 24, wherein the co-crystal is characterized by a melting point in a range of 198-202° C.

26. The co-crystal of claim 1, wherein the epicatechin is (+) epicatechin and the co-crystal-former of Formula (I) is L-proline.

27. The co-crystal of claim 26, wherein the co-crystal is characterized by a melting point in a range of 195-198° C.

28. A pharmaceutical composition comprising a co-crystal of any one of claims 1-27, and a pharmaceutically acceptable excipient.

29. A method of treating a disease or disorder that would benefit from modification of Electron Transport Chain, optionally complex IV, in a subject in need thereof, comprising administering to the subject a therapeutically effective amount of a co-crystal of any one of claims 1-27 or a pharmaceutical composition of claim 28.

30. The method of claim 29, wherein a disease or disorder is selected from the group consisting of impaired cognition, neurodegenerative diseases such as Alzheimer's or Leigh syndrome, dystonia, sarcopenia, cardiomyopathy of aging or other diseases associated with mitochondrial dysfunction, ischemic vascular disease, immunodeficiency states, ataxia, pulmonary inflammation and fibrosis, infantile encephalomyopathy, epilepsy, Charcot-Marie-Tooth disease, exocrine pancreatic insufficiency, impaired wound healing, and growth of cancer cells.

31. A method of lowering triglyceride levels in a subject in need thereof, comprising administering to the subject a therapeutically effective amount of a co-crystal of any one of claims 1-27 or a pharmaceutical composition of claim 28.

32. A method of treating a disease or disorder selected from the group consisting of a metabolic syndrome, Type II diabetes, a congenital hyperlipidemia, and drug-induced hyperlipidemia in a subject in need thereof, comprising administering to the subject a therapeutically effective amount of a co-crystal of any one of claims 1-27 or a pharmaceutical composition of claim 28.

33. A method of treating a condition related to mitochondrial dysfunction in a subject in need thereof, comprising administering to the subject a therapeutically effective amount of a co-crystal of any one of claims 1-27 or a pharmaceutical composition of claim 28.

34. A method for enhancing sports performance, endurance, building muscle shape or strength, or facilitating recovery from the effects of training or competition, in a subject in need thereof, comprising administering to the subject a therapeutically effective amount of a co-crystal of any one of claims 1-27 or a pharmaceutical composition of claim 28.

35. A method for treating or preventing dystrophinopathy, sarcoglycanopathy, or dysferlinopathy in a subject in need thereof, comprising administering to the subject a therapeutically effective amount of a co-crystal of any one of claims 1-27 or a pharmaceutical composition of claim 28.

36. A process for preparing a co-crystal of any one of claims 1-27, comprising the following steps:
(i) dissolving epicatechin and a co-crystal former of Formula (I) in a solvent to obtain a solution;
(ii) heating the solution obtained from step (i);
(iii) resting the heated solution of step (ii); and
(iv) collecting the co-crystal.

37. A co-crystal of any one of claims 1-27 for use as therapeutically active substance.

38. A co-crystal of any one of claims 1-27 or a pharmaceutical composition of claim 28 for use in treating a disease or disorder that would benefit from modification of Electron Transport Chain, for use in treating a condition related to mitochondrial dysfunction, or for use in treating or preventing dystrophinopathy, sarcoglycanopathy or dysferlinopathy.

39. Use of a co-crystal of any one of claims 1-27 or a pharmaceutical composition of claim 28 for the preparation of a medicament for the treatment of a disease, disorder or condition.
